(19) United States
(12) Patent Application Publication
(10) Pub. No.: US 2019/0070183 A1
(43) Pub. Date: Mar. 7, 2019

(54) Targeting Casein Kinase-1 and PI3K/AKT/mTOR Pathways for Treatment of c-Myc-Overexpressing Cancers, Organ Transplant Associated Complications and Autoimmune Diseases

(71) Applicant: The Trustees of Columbia University in the City of New York, New York, NY (US)

(72) Inventors: Changchun Deng, Jericho, NY (US); Shi-Xian Deng, White Plains, NY (US); Donald W. Landry, New York, NY (US); Mark Lipstein, New York, NY (US); Michael Mangone, Brooklyn, NY (US); Owen O'Connor, Scarsdale, NY (US); Xavier O. Jirau Serrano, Brooklyn, NY (US); Luigi Scotto, Stamford, CT (US); Xiaoming Xu, Fair Lawn, NJ (US)

(21) Appl. No.: 15/773,430

(22) PCT Filed: Nov. 4, 2016
(86) PCT No.: PCT/US16/60530

§ 371 Date: May 3, 2018

Related U.S. Application Data
(60) Provisional application No. 62/251,040, filed on Nov. 4, 2015.
(60) Provisional application No. 62/336,214, filed on May 13, 2016.

(51) Int. Cl. A61K 31/52; A61K 31/551; A61P 9/10; A61P 35/02; A61K 31/336; A61K 31/5375

(52) U.S. Cl. CPC A61K 31/52; A61K 31/551; A61K 31/5375; A61P 35/02; A61K 31/336; A61P 9/10

(57) ABSTRACT

The invention relates to the co-administration of select proteasome and PI3K inhibitors is useful for treating c-Myc-overexpressing cancers, particularly hematological cancers such as aggressive B- and T-cell lymphomas. In exemplified embodiments, coadministration of a dual PI3K/CK-1 inhibitor with a proteasome inhibitor synergistically increases cell death of aggressive B- and T-cell lymphomas as well as multiple myeloma over the individual or additive effect of either or both agents. This synergistic effect is associated with the previously unknown inhibition of the kinase casein kinase 1 epsilon (CK-1ε) by a PI3K inhibitor, such as TGR-1202. Accordingly, use of PI3K inhibitors that possess CK-1ε inhibition in combination with proteasome inhibitors provides a new therapy regime for treating c-Myc-overexpressing cancers, and particularly hematological cancers.

### CROSS-REFERENCE TO RELATED APPLICATIONS

This application claims benefit of: Provisional Appln. 62/251,040, filed Nov. 4, 2015 and Provisional Appln. 62/336,214, filed May 13, 2016 under 35 U.S.C. § 119(e), the entire contents of each of which are hereby incorporated by reference as if fully set forth herein.

### BACKGROUND

Treatment of hematological cancers such as myelomas, lymphomas and leukemias is very complex. Tremendous clinical variability among remissions is also observed in hematological cancer subjects, even those that occur after one course of therapy. Subjects who are resistant to therapy have very short survival times, regardless of when the resistance occurs. A need exists for an effective means to treat hematological cancer and to improve the efficacy of current chemotherapies in those subjects resistant, refractory, or otherwise not responsive to treatment with such chemotherapies.

c-Myc is a master transcription factor and one of the most frequently altered genes across a vast array of human cancers [1]. Overexpression of c-Myc is observed in up to 30% of cases of diffuse large B-cell lymphoma (DLBCL) [2], the most common type of aggressive lymphoma. c-Myc overexpression in lymphoma is a relatively common, and highly unfavorable, pathogenetic factor in DLBCL. Strategies that target this pathway could markedly improve the outcome of patients with c-Myc-overexpressing lymphomas and other hematologic cancers. To date no drugs that directly target c-Myc have been approved for the treatment of any cancer. In fact, since c-Myc is involved in many essential functions in normal cells, direct c-Myc inhibitors may theoretically be associated with significant toxicity. Alternatively, it may be advantageous to inhibit upstream cancer-specific signals that converge on c-Myc as a therapeutic strategy to mitigate the poor risks associated with c-Myc dysregulation in lymphoma.

### SUMMARY OF INVENTION

Certain embodiments described herein pertain to novel compositions, compounds, and therapeutic methods that are based on the discovery that targeting CK-1 alone or in conjunction with targeting the PI3K-AKT-mTOR signaling pathway provides improved outcomes in treating c-Myc-overexpressing cancers. In one embodiment, provided is a method for treating a c-Myc-overexpressing cancer in a subject comprising co-administering a therapeutically effective amount of a dual PI3K/CK-1 inhibitor with a therapeutically effective amount of a proteasome inhibitor, or optionally, co-administering a therapeutically effective amount of a PI3K inhibitor, a CK-1 inhibitor and a proteasome inhibitor. The cancer may be a hematological cancer or solid tumor in an organ selected from the group consisting of the lung, breast, prostate, ovary, colon, kidney, and liver. In a particular embodiment, the PI3K inhibitor is TGR-1202, or a therapeutically active analog or derivative thereof, or pharmaceutically acceptable salt of any of the foregoing. Moreover, the proteasome inhibitor is carfilzomib, or a therapeutically active analog or derivative thereof, or pharmaceutically acceptable salt of any of the foregoing. Typically, the dual PI3K/CK-1 inhibitor comprises CK-1ε, CK-1α, or CK-1δ inhibitory activity. In a specific embodiment, the dual PI3K/CK1 inhibitor comprises CK-1ε inhibitory activity. In another specific embodiment, CK-1 inhibitor inhibits CK-1ε, CK-1α, or CK-1δ.

According to another embodiment, provided is a method comprising: (a) determining a CK-1 expression level from a cancer cell sample obtained from a subject who has cancer; and (b) comparing the expression level from the cancer cell sample to an expression level of a control, wherein an elevated CK-1 expression level in the cancer cell sample relative to the control indicates that the cancer is susceptible to PI3K and CK-1 inhibition; and if the cancer is susceptible, co-administering a therapeutically effective amount of a dual PI3K/CK-1 inhibitor with a therapeutically effective amount of a proteasome inhibitor, or optionally, co-administering a therapeutically effective amount of a PI3K inhibitor, CK-1 inhibitor and proteasome inhibitor. In a specific embodiment of this method, the dual PI3K/CK-1 inhibitor is TGR-1202 or CUX-03173, a therapeutically active analog or derivative thereof, or a pharmaceutically acceptable salt of any of the foregoing; and the proteasome inhibitor is carfilzomib, or a therapeutically active analog or derivative thereof, or a pharmaceutically acceptable salt of any of the foregoing. The cancer is typically a c-Myc-overexpressing cancer, such as B cell cancer (e.g. multiple myeloma or lymphoma). The expression level is selected from the group consisting of RNA transcript level and protein level. The CK-1 expression level may be of CK-1ε, CK-1α, or CK-1δ. In a specific embodiment, the CK-1 is CK-1α, and the cancer is selected from the group consisting of lung cancer, colon cancer, and liposarcoma. In another specific embodiment, the CK-1 is CK-1δ and the cancer is selected from the group consisting of lung cancer, choriocarcinoma, high-grade ductal pancreatic carcinoma and glioblastoma. In a primary embodiment, the CK-1 is CK-1ε and the cancer is selected from the group consisting of B cell cancer, lung cancer, breast cancer, adenoid cystic carcinoma, epithelial ovarian cancer, renal cancer, bladder cancer, prostate cancer, melanoma and seminoma.

Another embodiment involves screening for PI3K inhibitors that have CK-1 inhibitory activity. The method involves contacting a known PI3K inhibitor candidate agent with a CK-1 isoform, to produce a test sample; determining level of CK-1 isoform activity in test sample; and if the CK-1 isoform activity is reduced, selecting the PI3K candidate agent as having a dual function of also inhibiting CK-1.

A further embodiment pertains to a pharmaceutical formulation comprising: a therapeutically effective amount of a dual PI3K/CK-1 inhibitor; and a therapeutically effective amount of a proteasome inhibitor; and optionally a pharmaceutically acceptable carrier. In a specific embodiment, the proteasome inhibitor is carfilzomib, or a therapeutically active analog or derivative thereof, or a pharmaceutically acceptable salt of any of the foregoing.

Another pharmaceutical formulation provided herein comprises a therapeutically effective amount of a PI3k inhibitor; a therapeutically effective amount of a CK-1 inhibitor; and a therapeutically effective amount of a proteasome inhibitor; and optionally a pharmaceutically acceptable carrier.

Moreover, pharmaceutical formulations are disclosed comprising: (i) a therapeutically effective amount of a dual PI3K/CK-1 inhibitor and therapeutically effective amount of a proteasome inhibitor; ii) a therapeutically effective amount of a PI3K-AKT-mTOR signaling pathway inhibitor inhibitor, a therapeutically effective amount of a CK-1 inhibitor, and a therapeutically effective amount of a proteasome inhibitor; iii) a therapeutically effect amount of a dual PI3K/CK-1 inhibitor, a therapeutically effect amount of a CK-1 inhibitor and a therapeutically effect amount of proteasome inhibitor; iv) a therapeutically effect amount of a dual PI3K/CK-1 inhibitor and a therapeutically effect amount of an adjunct cancer therapeutic agent (excluding a proteasome inhibitor); or v) a therapeutically effect amount of a PI3K-AKT-mTOR signaling pathway inhibitor, a therapeutically effect amount of a CK-1 inhibitor and a therapeutically effect amount of an adjunct cancer therapeutic agent (excluding a proteasome inhibitor); and optionally, wherein i-v are further combined with a pharmaceutically acceptable carrier.

Certain method embodiments are disclosed that involve targeting CK-1 for treatment of c-Myc overexpressing cancers. For example, disclosed is method for treating a c-Myc-overexpressing cancer in a subject comprising administering a c-Myc reducing amount of a CK-1 epsilon inhibitor or a dual PI3K/CK-1 inhibitor, or both; and optionally co-administering a therapeutically effective amount of a proteasome inhibitor or a PI3K inhibitor, or both. The cancer may be a hematological cancer, such as a B cell cancer (e.g. multiple myeloma or lymphoma). Alternatively, the cancer is cancer solid tumor in an organ selected from the group consisting of the lung, breast, prostate, ovary, colon, kidney, and liver. In a specific embodiment, the PI3K inhibitor comprises Idelalisib or develisib, or a therapeutically active therapeutically active analog or derivative thereof, or pharmaceutically acceptable salt thereof of the foregoing. Moreover, the dual PI3K/CK-1 inhibitor is selected from the group consisting of TGR-1202 and CUX-03173; or a therapeutically active therapeutically active analog or derivative thereof, or pharmaceutically acceptable salt thereof of the foregoing. Further still, the proteasome inhibitor comprises carfilzomib, or an therapeutically active analog or derivative thereof, or pharmaceutically acceptable salt thereof of the foregoing. Typically, the CK-1 inhibitor comprises CK-1ε, CK-1α, or CK-1δ inhibitory activity, or a combination thereof, an in specific embodiments, the CK-1 inhibitor comprises CK-1ε inhibitory activity.

Other embodiments disclosed herein involve administration of agents discovered to have particular benefit in treat c-Myc-overexpressing cancers. For example, the method involves administering an agent according to Formula III or Formula IV, or a pharmaceutically acceptable salt thereof:

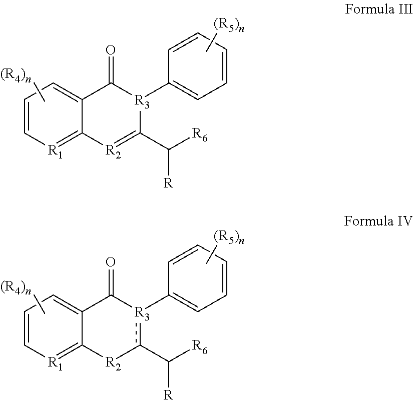

wherein
R is H or any one of groups A-G:

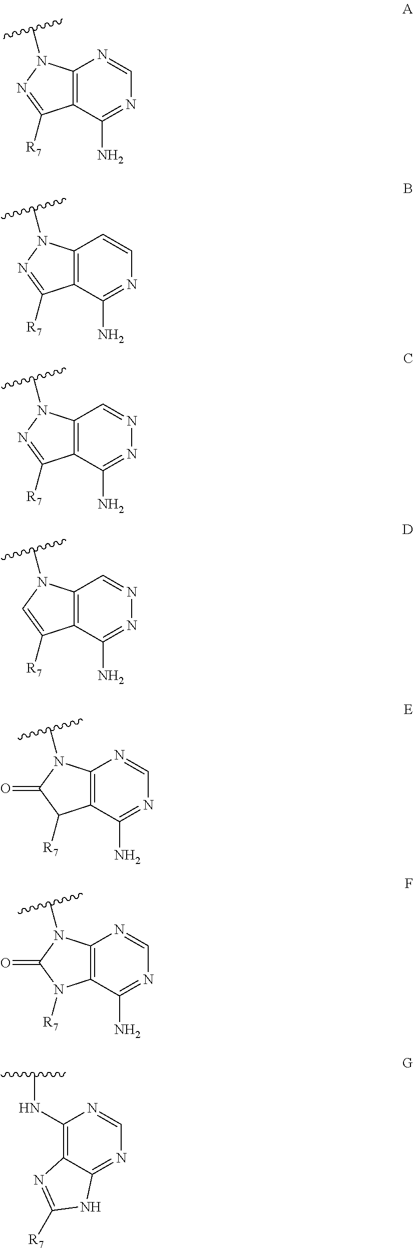

and wherein [symbol]
represents a single or double bond;
R₁ is CH, substituted C or N;

### R₂

in the compound of Formula III is CH, substituted C or N;

in the compound of Formula IV is O, CH₂, substituted C, NH or substituted N;

### R₃

in the compound of Formula III is CH, substituted C or N;

in the compound of Formula IV is
CH, substituted C or N when [symbol] represents a single bond; or
C when [symbol] represents a double bond;
   each R₄ is independently substituted alkyl, unsubstituted alkyl, substituted alkenyl, unsubstituted alkenyl, substituted alkynyl, unsubstituted alkynyl, or halogen;
   each R₅ is independently substituted alkyl, unsubstituted alkyl, substituted alkenyl, unsubstituted alkenyl, substituted alkynyl, unsubstituted alkynyl, or halogen;
   R₆ is H, Me or Me substituted with halogen;
   R₇ is H or a group selected from any one of groups J, K and H

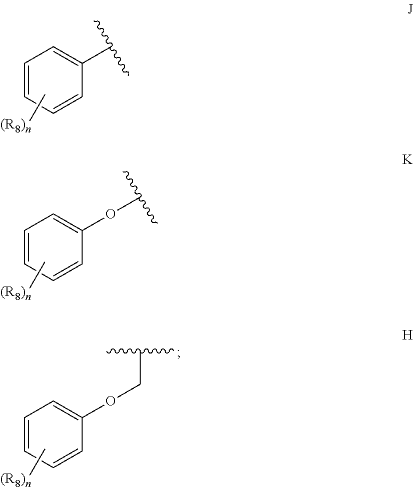

and
each R₈ is independently substituted alkyl, unsubstituted alkyl, substituted O-alkyl, unsubstituted O-alkyl or halogen;
n,

for R₄ and when R₁ is not N, is 0, 1, 2, 3 or 4;

for R₄ and when R₁ is N, is 0, 1, 2 or 3;

for R₅ is 0, 1, 2, 3, 4 or 5;

for R₈ is 0, 1, 2, 3, 4 or 5;

wherein the compound according to Formula III or Formula IV is administered at a CK-1 reducing effective amount.

Certain specific agents used in the preceding method involve further features:

(i) the proviso that compounds of formula III wherein at the same time R is group A, R₁ is CH, R₃ is N and R₇ is J, are excluded;

(ii) the proviso that compounds of formula IV wherein at the same time R is group A, R₁ is CH, R₂ is O, R₃ is C, [symbol] represents a double bond, and R₇ is J, are excluded;

(iii) the proviso that R₇ is not H when R is group G;

(iv) the provisos that
compounds of formula III wherein at the same time R is group A, R₁ is CH, R₃ is N and R₇ is J, are excluded;
compounds of formula IV wherein at the same time R is group A, R₁ is CH, R₂ is O, R₃ is C, [symbol] represents a double bond, and R₇ is J, are excluded; and/or
R₇ is not H when R is group G.

(v) wherein R₁ is N.

(vi) wherein R₂ is not O.

(vii) wherein R₃ is not N.

(viii) wherein R₄ is halogen and n for R₄ is 1 or 2.

(ix) wherein R₄ is F and n for R₄ is 1 or 2;

(x) wherein R₄ is F, n for R₄ is 1, and R₄ is located at position 5 of the quinazolin-4-one ring to which it is attached;

(xi) wherein n for R₅ is 0;

(xii) wherein R₆ is Me;

(xiii) wherein R is not group A;

(xiv) wherein R is group A;

(xv) wherein R₇ is J;

(xvi) wherein R₇ is not J;

(xvii) wherein n for R₈ is 2, one R₈ is isopropyl or O-isopropyl, and the other R₈ is halogen, preferably F;

(xviii) wherein R₇ is one of the following:

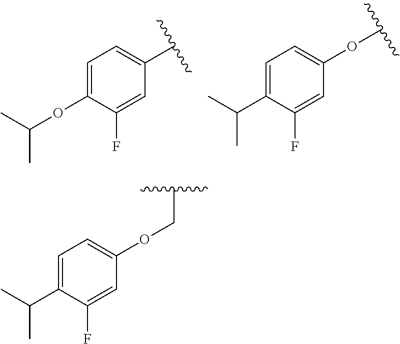 [SMILES: *c1ccc(OC(C)C)c(F)c1 ; *Oc1ccc(C(C)C)c(F)c1 ; *COc1ccc(C(C)C)c(F)c1]

In a specific embodiment, the CK-1 epsilon inhibitor is

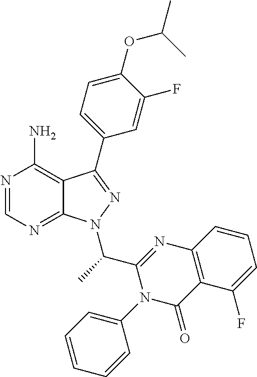 [SMILES: CC(C)Oc1ccc(-c2nn([C@@H](C)c3nc4cccc(F)c4c(=O)n3-c3ccccc3)c3ncnc(N)c23)cc1F]

Other embodiments involve comprising (a) determining a CK-1 expression level from a cancer cell sample obtained from a subject who has cancer; (b) comparing the expression level from the cancer cell sample to an expression level of a control, wherein an elevated CK-1 expression level in the cancer cell sample relative to the control indicates that the cancer is susceptible to CK-1 inhibition; and administering a therapeutically effective amount of a CK-1 inhibitor or dual PI3K/CK-1 inhibitor, or both, to a susceptible cancer. This method may further comprise co-administering a therapeutically effective amount of a proteasome inhibitor, or a therapeutically effective amount of a PI3K inhibitor, or both. In a specific embodiment, the cancer is a c-Myc-overexpressing cancer, such as a B cell cancer (e.g. multiple myeloma or lymphoma). The expression level determined may be selected from the group consisting of RNA transcript level and protein level. The CK-1 relevant to this method may be selected from the group consisting of CK-1ε, CK-1α, or CK-1δ. In a specific embodiment, the CK-1 is CK-1α, and the cancer is selected from the group consisting of lung cancer, colon cancer, and liposarcoma. Alternatively, the CK-1 is CK-1δ and the cancer is selected from the group consisting of lung cancer, choriocarcinoma, high-grade ductal pancreatic carcinoma and glioblastoma. In primary embodiments, the CK-1 is CK-1ε and the cancer is selected from the group consisting of B cell cancer, lung cancer, breast cancer, adenoid cystic carcinoma, epithelial ovarian cancer, renal cancer, bladder cancer, prostate cancer, melanoma and seminoma.

A further method embodiment involves (a) determining a pre-treatment CK-1 expression level in a first cancer cell sample from a subject that has cancer; (b) co-administering a therapeutically effective amount of a dual PI3K/CK-1 inhibitor with a therapeutically effective amount of a proteasome inhibitor, or optionally, co-administering a therapeutically effective amount of a PI3K inhibitor, CK-1 inhibitor and proteasome inhibitor; and (c) determining a post-treatment CK-1 expression level in a second cancer cell sample from the subject. A reduction in the post-treatment CK-1 expression level relative to the pre-treatment level indicates that the co-administration chemotherapy is effective to treat the cancer. The CK-1 is typically selected from the group consisting of CK-1ε, CK-1α, or CK-1δ. The method may further involve repeating step (b) if a reduction in the post-treatment CK-1 expression level is determined.

Other embodiments pertain to new compounds according to Formula III or Formula IV:

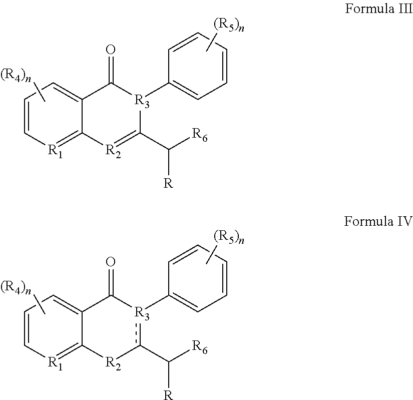

wherein
R is H or any one of groups A-G:

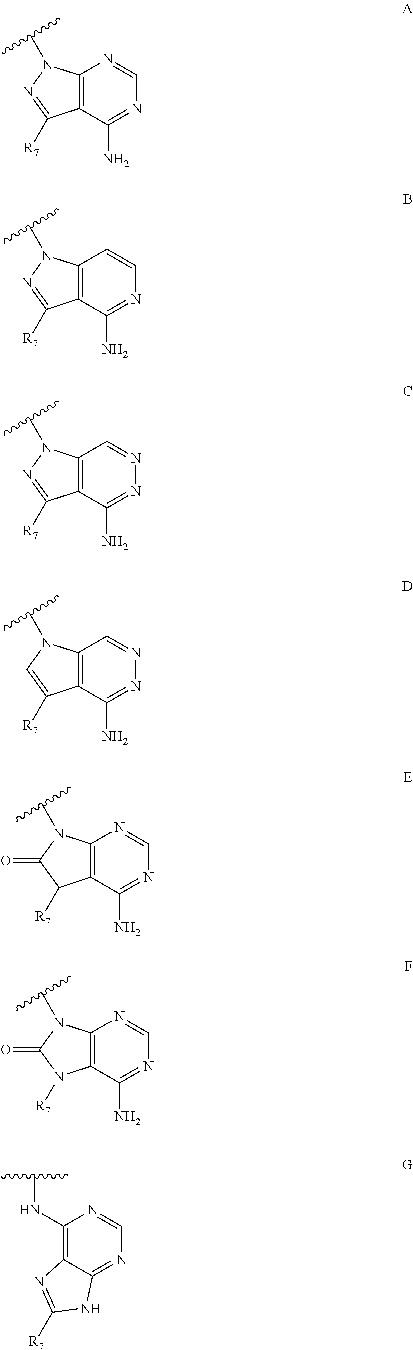

and wherein [symbol]
represents a single or double bond;
R₁ is CH, substituted C or N;

### R₂

in the compound of Formula III is CH, substituted C or N;

in the compound of Formula IV is O, CH₂, substituted C, NH or substituted N;

### R₃

in the compound of Formula III is CH, substituted C or N;

in the compound of Formula IV is
CH, substituted C or N when [symbol] represents a single bond; or
C when [symbol] represents a double bond;
   each R₄ is independently substituted alkyl, unsubstituted alkyl, substituted alkenyl, unsubstituted alkenyl, substituted alkynyl, unsubstituted alkynyl, or halogen;
   each R₅ is independently substituted alkyl, unsubstituted alkyl, substituted alkenyl, unsubstituted alkenyl, substituted alkynyl, unsubstituted alkynyl, or halogen;
   R₆ is H, Me or Me substituted with halogen;
   R₇ is H or a group selected from any one of groups J, K and H

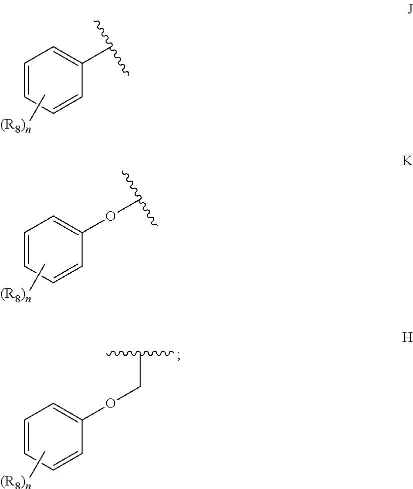

and
each R₈ is independently substituted alkyl, unsubstituted alkyl, substituted O-alkyl, unsubstituted O-alkyl or halogen;
n,

for R₄ and when R₁ is not N, is 0, 1, 2, 3 or 4;

for R₄ and when R₁ is N, is 0, 1, 2 or 3;

for R₅ is 0, 1, 2, 3, 4 or 5;

for R₈ is 0, 1, 2, 3, 4 or 5;

further comprising the provisos that

(i) compounds of formula III wherein at the same time R is group A, R₁ is CH, R₃ is N and R₇ is J, are excluded;

(ii) compounds of formula IV wherein at the same time R is group A, R₁ is CH, R₂ is O, R₃ is C, [symbol] represents a double bond, and R₇ is J, are excluded;

(iii) R₇ is not H when R is group G.

In specific agents related to formulas III and IV wherein R₁ is N, the agent includes:
(i) wherein R₂ is not O;
(ii) wherein R₃ is not N
(iii) wherein R₄ is halogen and n for R₄ is 1 or 2;
(iv) wherein R₄ is F and n for R₄ is 1 or 2;
(v) wherein R₄ is F, n for R₄ is 1, and R₄ is located at position 5 of the quinazolin-4-one ring to which it is attached;
(vi) wherein n for R₅ is 0;
(vii) wherein R₆ is Me;
(viii) wherein R is not group A;
(ix) wherein R is group A;
(x) wherein R₇ is J;
(xi) wherein R₇ is not J;
(xii) wherein n for R₈ is 2, one R₈ is isopropyl or O-isopropyl, and the other R₈ is halogen, preferably F;
(xiii) wherein R₇ is one of the following:

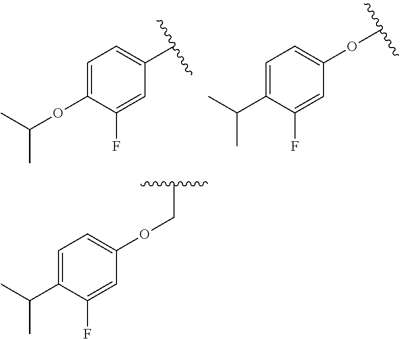 [SMILES: *c1ccc(OC(C)C)c(F)c1 ; *Oc1ccc(C(C)C)c(F)c1 ; *COc1ccc(C(C)C)c(F)c1]

Also provided is a kit for administering a first and a second pharmaceutical composition to a subject suffering from a c-Myc-overexpressing cancer, the kit comprising: i) a plurality of separate containers, the contents of at least two containers differing from each other in whole or in part, wherein at least one of such containers contains a CK-1 inhibitor or a dual PI3K/CK-1 inhibitor, or both, with or without additional pharmaceutical carrier or diluent, and at least one different container contains a proteasome inhibitor, with or without additional pharmaceutical carrier or diluent; or at least one different container contains a PI3K inhibitor, with or without additional pharmaceutical carrier or diluent and, optionally, ii) instructions for the use of the contents of the containers after an interval of time has passed after administration of the first pharmaceutical composition for the treatment of a subject suffering from a hematological cancer.

Also disclosed are methods that involve CK-1 inhibition as a lead-in therapy for an adjunct cancer therapy protocol. In one example, the method comprises administering to a c-Myc-overexpressing cell in a subject a c-myc reducing amount of a CK-1 inhibitor or dual PI3K/CK-1 inhibitor; and administering an adjunct cancer therapy protocol in the subject. In a specific example, the adjunct cancer therapy protocol comprises co-administration of an adjunct cancer therapeutic agent. Moreover, the adjunct cancer therapeutic agent may be co-administered upon reduction of c-Myc in the c-Myc-overexpressing cell by CK-1 inhibitor administration. In a more specific embodiment, the adjunct cancer therapeutic agent excludes a proteasome inhibitor.

Also provided is a method comprising: administering to a subject a therapeutically effective amount of a CK-1epsilon inhibitor; or co-administering (i) a therapeutically effective amount of an mTOR inhibitor and a therapeutically effective amount of a proteasome inhibitor; (ii) a therapeutically effective amount of an mTOR inhibitor and a therapeutically effective amount of a CK-1epsilon inhibitor; or (iii) a therapeutically effective amount of CK-1epsilon inhibitor and a therapeutically effective amount of a proteasome inhibitor; wherein the subject has received an organ transplant. The subject is typically one that is at risk of GVHD related to the organ transplant, or exhibits symptoms of GVHD. In a specific example, the organ transplant is a bone marrow transplant.

Other methods comprise administering to a subject a therapeutically effective amount of an agent that inhibits CK-1epsilon; or co-administering (i) a therapeutically effective amount of an mTOR inhibitor and a therapeutically effective amount of a proteasome inhibitor; (ii) a therapeutically effective amount of an mTOR inhibitor and a therapeutically effective amount of an agent that inhibits CK-1epsilon; or (iii) a therapeutically effective amount of an agent that inhibits CK-1epsilon and a therapeutically effective amount of a proteasome inhibitor; wherein the subject exhibits symptoms of and/or has been diagnosed with an autoimmune disease. The autoimmune disease may include rheumatoid arthritis, psoriasis, eczema, asthma, multiple sclerosis, inflammatory bowel disease, Chrohn's disease, colitis, systemic lupus erythematosus, myasthenia gravis, Sjögren's syndrome and sclerodema, autoimmune hemolytic anemia, cold agglutinin disease, or IgA nephropathy.

These and other embodiments are described further herein.

### BRIEF DESCRIPTION OF THE DRAWINGS

The present invention is illustrated by way of example, and not by way of limitation, in the figures of the accompanying drawings.

FIG. 1. TGR-1202 is a selective PI3Kδ inhibitor. FIG. 1A: Structure of TGR-1202, in comparison to idelalisib/Cal-101. FIG. 1B: Cell free PI3K activity assay. Potency of TGR-1202 against the human δ isoform of PI3K was evaluated in a HTRF based enzyme assay in the presence of ATP at 100 μM. The IC50 was 22 nM. FIG. 1C: Potency of TGR-1202 against the other three isoforms, namely, α, β, and γ was determined as in FIG. 1B, and their IC50 values calculated. Selectivity of the 6 over the other isoform was expressed as the ratio of IC50_{(α, β, or γ)}/IC50_{(δ)}. FIG. 1D: Cell based PI3K activity assay. Potency of TGR-1202 and Cal-101 against PI3Kδ was determined in an anti-IgM induced human B cell proliferation assay. RP5264 is the non-pharmaceutical equivalent of TGR-1202.

FIG. 2. Drug: drug interaction between proteasome inhibitors and PI3K inhibitors. FIG. 2A: DLBCL cell line LY10 was treated for 24 hours with the indicated drugs and concentrations as single agents and in combinations. Viable cells were quantitated by CellTiter Glo assay (Promega). The percentage of growth inhibition is calculated as (1-viable cells in the treated sample/viable cells in the untreated control). Data from four combination pairs were presented. FIG. 2B: Synergy of proteasome inhibitors and PI3K inhibitors. Synergy indices were calculated by EOB values using the results from FIG. 2A based on the Bliss model of additivism as described in Methods, for each of the 100 combination conditions of the four combination pairs.

FIG. 3. Synergy of TGR-1202 and carfilzomib in lymphoma and myeloma. (FIG. 3A-FIG. 3K) Cells representing diverse histological subtypes of lymphoma and myeloma were treated by TGR-1202 (TG), Cal-101 (Cal), carfilzomib (Cfz), and bortezomib (Bort), as single agents and in combination for 24 hours. The percentage of growth inhibition is calculated as above in FIG. 2. The expected inhibition is calculated using the Bliss model of additivism as described in Methods. FIG. 3L-N: PARP cleavage. LY10 (FIG. 3L), LY7 (FIG. 3M) and T-ALL PF382 (FIG. 3N) cells were treated with the indicated drugs for 24 h, and harvested and processed for Western blot. FIG. 3O: Activation of caspase 3/7. The DLBCL cell line LY10 was treated by the indicated drugs for 24 h, then analyzed for caspase 3/7 activity. PI3Ki, PI3K inhibitors; Pi: proteasome inhibitor. Results for Cal-101, bortezomib, and their combinations were presented in hashed dark colored bars; those for TGR-1202, carfilzomib, and their combinations in dotted lighter colored bars.

FIG. 4. Effects of PI3K and proteasome inhibitors on the PI3K-AKT-mTOR-eIF4F-Myc signal cascade. LY10 (FIG. 4A), LY7 (FIG. 4B), and PF382 (FIG. 4C) cells were treated with the indicated drugs as single agents or in combinations for 24 h, then processed for Western blot using the antibodies against the indicated (phosphorylated) proteins. Cfz: carfilzomib, TG: TGR-1202, bort: bortezomib, Cal: Cal-101/idelalisib.

FIG. 5. Effects of PI3K and proteasome inhibitors on the expression of c-Myc and Myc target genes. FIG. 5A: LY10 cells were treated at the indicated conditions for 24 h, the process for Western blot using the anti-c-Myc antibody. FIGS. 5B and 5C: Same samples from FIG. 5A were also processed for RNA extraction and qPCR, using primers for c-Myc (FIG. 5B), LDH-A and PKM (FIG. 5C). The internal controls were cyclophilin A and GAPDH. FIG. 5D: Schema of a bicistronic luciferase reporter for the translation of c-Myc. This plasmid was transfected into the DLBCL cell line LY7. FIGS. 5E-5F: LY7 cells were treated at the indicated conditions, and processed for Western blot (FIG. 5E) and qPCR (FIG. 5F) to determine the levels of the c-Myc protein and mRNA respectively. FIG. 5G: LY7 cells were transiently transfected by the reporter plasmid from FIG. 5D. After overnight recovery from electroporation, cells were treated at the indicated conditions for 24 h. Renilla and firefly luciferase signals were measured as in Methods. R/F luc ratios from the treatment groups were compared with the untreated control, which was arbitrarily set as 100%.

FIG. 6. Pharmacological activities of PI3K and proteasome inhibitors in primary lymphoma cells. FIGS. 6A-6F. Cytotoxicity. Primary cells were isolated by ficoll gradient separation from three patients with SLL (FIG. 6A & FIG. 6B), CLL (FIG. 6C & FIG. 6D), MCL (FIG. 6E & FIG. 6F) respectively. The SLL cells were from pleural fluid, and the CLL and MCL cells were from peripheral blood. The cells were incubated with the experimental drugs as single agents and in combinations for 48 hours. The viability was determined by Cell Titer Glo, and presented as a function of each treatment conditions, as a percentage of the untreated control. FIG. 6G & FIG. 6H: Western blot. Cells from the SLL and CLL patients were treated as above, and collected for Western blot at the end of 48 h treatment. FIG. 6I: Peripheral blood mononuclear cells were isolated from a healthy donor and treated by the indicated drug combinations for 24-72 h. Viability was calculated as the percentage of live cells in the treated versus untreated control samples.

FIG. 7. Effects of PI3Kδ and proteasome inhibitors on c-Myc dependent gene transcription. (A-E) LY10 cells were treated by vehicle control, TGR-1202 (“T”), idelalisib (“I”), carfilzomib (“C”), bortezomib (“B”), and the 4 combinations including TC, TB, IC, and IB for 24 h then processed for RNA-seq to determine mRNA transcription. (FIG. 7A-FIG. 7D) Changes in gene expression relative to the vehicle treated control were ranked listed and used to perform GSEA analysis of target gene sets (GS) of transcription factors using the Molecular Signatures Database (MSigDB). (FIG. 7A) GSEA of c-Myc target genes included GS52: Schuhmacher_myc_targets_up; GS72: Dang_myc_targets_up; GS70: Dang_regulated_by_myc_up; GS32: Kim_myc_amplification_targets_up; GS29: Schlosser_myc_targets_and_serum_response. (FIG. 7B) GSEA of E2F target genes included GS43: Kalma_E2F1_targets (11 in gene set); GS38: Ren_bound_by_E2F (61 in gene set); GS22: SGCGSSAAA_V$E2F1DP2_01 (168 in gene set). (FIG. 7C) Differential by chi². (FIG. 7D) Partial summary of gene set enrichment. The number of genes in each set (n), the normalized enrichment score (NES), and test of statistical significance (FDR q value) were listed. (FIG. 7E) Genes uniquely regulated (up- or down-) by the 4 combinations TC, TB, IC, and IB but not the respective contributing single agents were first identified, then rank according to their expression levels specific to the combination effect (Details in Methods). GSEA analysis was performed on 2 representative c-Myc gene sets including GS52 and GS70. (FIG. 7F) LY7 cells were treated as indicated for 24 h then processed for Western blot against two known targets of c-Myc, namely eIF4B and E2F1.

FIG. 8 shows that TGR1202 is equivalent to the combination of CAL-101 and PF-4800567/2 in reducing the viability of lymphoma cells. LY10 cells were treated with the indicated drugs as single agents or in combinations for 24 h.

FIG. 9 shows that TGR-1202 or the Combination of CAL-101 and PF-4800567/2 is Required for Sustained Inhibition of Phosphorylation of 4EBP1 and Synthesis of c-Myc.

FIG. 9A: LY10 cells were treated with the indicated drugs as single agents or in combinations for 12 h (left) and 24 h, then processed for Western blot using the antibodies against the indicated (phosphorylated) proteins. FIG. 9B shows the effect of the noted agent treatment on c-Myc expression as a percent of control.

FIG. 10 is a diagram representing a model of how targeting the intricate networks of PI3K-AKT-mTOR, CK-1ε, CK-1α, lead to decreased c-Myc expression.

FIG. 11. Overexpression of eIF4E suppresses the potent synergy of TGR-1202 and carfilzomib. Myeloma cell line H929 transduced by lentivirus with an eIF4E overexpressing plasmid (eIF4E) or an empty vector (EV) and cells with no transduction (No TDX) were treated for 24 h, and checked for viability (FIG. 11A) and levels of c-Myc and eIF4E (FIG. 11B).

FIG. 12. TGR-1202 and the CK1ε inhibitor PF4800567 share functional and structural similarity. (FIG. 12A) A partial summary of kinome profiling focusing on various casein kinases. The assay was performed by Reaction Biology. The drugs were studied at 1 μM. The values indicated residual kinase activity after treatment by the study drugs. (FIG. 12B) Structures of TGR-1202 in comparison to the CK1ε inhibitor PF4800567, idelalisib, and newly synthesized analogs of TGR-1202 including CUX-03166 and CUX-03173. The central pyrazolopyperazine amine moiety is circled in blue, and the ring atoms' numbering is indicated. The arrows denote the positions involved as hydrogen bonds donor (amine group) and acceptor (position 1). (FIG. 12C & FIG. 12E) X-ray crystallography and binding interaction map of CK1ε and PF4800567. (FIG. 12D & FIG. 12F) In silico docking of TGR-1202, CUX-03173, and CUX-03166 into the ATP binding pocket of CK1ε. The legend for the interaction maps (FIG. 12E and FIG. 12F) is given at the bottom of panel D. (FIG. 12G) Cell free kinase assay of CK1ε in the presence of PF4800567, TGR-1202, idelalisib, CUX-03166 and CUX-03173 using CK1ε enzyme system and ADP-Glo™ Kinase Assay from Promega.

FIG. 13. Inhibition of CK1ε is an important mechanism for TGR-1202 to silence c-Myc. (FIG. 13A) Cell based assay of CK1ε activity measured by its autophosphorylation. LY7 cells were pretreated with one of the indicated drugs (PF4800567, TGR-1202, Idelalisib, and CUX-03173) for 1 h then treated by the phosphatase inhibitor calyculin A for 0-60 min. Cells were then lysed and proteins extracted for Western blot. The upward mobility shift of CK1ε indicates it is auto-phosphorylated. (FIG. 13B) LY7 cells were treated by idelalisib, TGR-1202, and PF4800567 for 24 h then viability was measured by Cell Titer Glo. (FIG. 13C) LY7 was stably transfected with the reporter plasmid in FIG. 5G and treated with the drugs for 24 h. Renilla and firefly luciferase signals were measured. R/F luc ratios represents the efficiency of eIF4F dependent translation downstream of the endogenous 5′UTR of c-Myc. (FIG. 13D-FIG. 13F) LY7 cells were treated by the indicated drugs as single agents for 6 h (FIG. 13D) or 24 h (FIG. 13E & FIG. 13F) then processed for Western blot. (FIG. 13G) LY7 and LY10 cells were treated by TGR-1202 or CUX-03173 for 24 h then processed for Western blot. PF: PF4800567, TG: TGR-1202, Ide: idelalisib, CUX: CUX-03173.

FIG. 14. In silico docking. (FIG. 14A) Top-scoring binding pose of CUX-03173 (blue) superposed with the proposed binding pose of TGR-1202 (green). (FIG. 14B) Proposed docked binding mode of CUX-03173 into the ATP-binding pocket of CK1ε.

FIG. 15 In silico docking. (FIG. 15A) Interaction map of CUX-03173 in its proposed binding mode with CK1ε ATP-binding pocket. The legend for the interaction map is indicated at the bottom of the panel. (FIG. 15B) CUX-03166 structure superposed on the CUX-03173 in its proposed binding mode to CK1ε. The residues that form the floor of the CK1ε ATP-binding pocket below the position of the central methyl group of CUX-03173 (i.e. Asp 132, Leu 135 and Ile 148) are represented in van der Waals sphere (hydrogen atoms not represented), and the surface of the pocket floor at this position is also represented.

FIG. 16. The PI3Kδ inhibitor TGR-1202 is active in lymphoma models and in patients. (FIG. 16A) The structural formulae of TGR-1202 and Idelalisib with the active quinazolinone moieties highlighted. (FIG. 16B) Cell-free in vitro kinase assay of TGR-1202 against the PI3Kδ isoform. (FIG. 16C) Cell-based assay measuring inhibition of S473 p-AKT in serum-starved leukemia and lymphoma cell lines at 4 h. (FIG. 16D) Response of the subcutaneously xenograft model of T-ALL to 3 treatments, including vehicle control, TGR-1202 (150 mg/kg), and ara-C (50 mg/kg daily), over 25 days. The xenograft was derived from the MOLT-4 cell line in NOD/SCID mice. (FIG. 16E) Pre- and Post-treatment CT scans of 2 DLBCL patients on a clinical study of TGR-1202.

FIG. 17. The PI3Kd inhibitor TGR-1202 is active in lymphoma models and in patients. (FIG. 17A) Comparison of TGR-1202 and idelalisib for their targeting selectivity of the PI3K isoforms, based on cell free assay of PI3 kinase. (FIG. 17B) Comparison of TGR-1202 and idelalisib for their efficacy in DLBCL in clinical trials. (FIG. 17C) Pre- and Post-treatment CT scans of a DLBCL patient on a clinical study of TGR-1202.

FIG. 18. CK1ε and PI3K-mTOR play distinct and cooperative roles in translation via regulating 4EBP1, and have opposing effects on p70S6K1. (FIG. 18A) Western blot analysis of LY7 cells treated with Idelalisib (Ide), TGR-1202 (TG), and PF-4800567 (PF) at 0, 15, 25, 50 μM for 6 h. (FIG. 18B) Western blot analysis of LY7 cells treated by various singles agents and combinations for 24 h. For example, Ide10+PF10 indicates the combination of idelalisib at 10 μM and PF4800567 at 10 μM. (FIG. 18C) Schema of a bicistronic luciferase reporter for the translation of c-Myc. UTR: untranslated region of c-Myc, IRES: internal ribosome entry site of polio virus, Luc-R: renilla luciferase, Luc-F: firefly luciferase. (FIG. 18D) Results of the luciferase assay using the bicistronic reporter from (C). LY7 stably expressing the reporter was treated with the indicated drugs at 15, 25, and 50 μM for 24 hr. R/F luc ratios from the treatment groups were calculated as a percentage of the untreated control, and represents the efficiency of eIF4F cap-dependent translation regulated at the endogenous 5′UTR of c-Myc. (FIG. 18E) LY7 cells stably expressing a c-Myc mRNA without its endogenous 5′UTR (“Myc+++” or “M”) were treated at the indicated concentrations of TGR-1202 for 24 hr and compared via c-Myc blot and cell viability to the corresponding empty vector (“E”) and untransfected parental control (“C-”) cells. (FIG. 18F) Western blot comparing the response to 24 h PP242 treatment in the parental LY7 cells not infected by lentivirus (“P”) and LY7 cells infected with lentivirus harboring shRNA targeting CK1ε (“ck”) and 4EBP1 (“bp”). (FIG. 18G). Western blot comparing the effects of PP242 (PP) and PF4800567. Treatment was 24 h in LY7. (FIG. 18H) Western blot comparing PP242 as a single agent and in combinations. Treatment was 24 h in LY7. C-: untreated control, PP: PP242, TG: TGR-1202, Ide: Idelalisib, PF: PF-4800567. alone and in combination for 24 hr. PP was at 0.25 μM and the other drugs were at 5 and 15 μM.

FIG. 19. CK1ε and PI3K-mTOR play distinct and cooperative roles in translation via regulating 4EBP1, and have opposing effects on p70S6K1. (FIG. 19A) Western blot analysis of LY7 cells treated with Idelalisib (Ide), TGR-1202 (TG), and PF-4800567 (PF) at 0, 15, 25, 50 μM for 24 h. (FIG. 19B) Western blot of LY7 cells treated by TGR-1202 or CUX-03173 for 24 h. PF: PF4800567, TG: TGR-1202, Ide: idelalisib, CUX: CUX-03173. (FIG. 19C) LY7 cells were treated by idelalisib, TGR-1202, and PF4800567 for 24 h then viability was measured by Cell Titer Glo. (FIG. 19D) Western blot of the parental LY7 cells (control) and LY7 cells transduced by shRNA targeting CK1ε (CSNK1E kd) or 4EBP1 (4EBP1 kd). (FIG. 19E-G) Responses of the parental LY7 cells (control or NTD) and LY7 cells transduced by shRNA targeting CK1 ε (shCK1ε) to TGR-1202 (TGR, in E), idelalisib/Cal-101 (Cal, in F), and PP242. Viability was measured by Cell Titer Glo after 24 h of treatment. (FIG. 19H) Quantitation of c-Myc protein level based on Western blot in LY7 cells of different genetic background treated by PP242 for 24 h. LY7 NTD: parental LY7 cells not infected by lentivirus, shCK1ε: LY7 cells infected with lentivirus harboring shRNA targeting CK1ε, sh4EBP1: LY7 cells infected with lentivirus harboring shRNA targeting 4EBP1. (FIG. 19I) Western blot comparing PP242 as a single agent and in combinations. Treatment was 6H in LY7. C-: untreated control, PP: PP242, TG: TGR-1202, Ide: Idelalisib, PF: PF-4800567. alone and in combination for 24 hr. PP was at 0.25 mM, and the other drugs were at 5 and 15 mM. (FIG. 19J) Viability of LY7 cells treated as by PP242 and TGR-1202 as single agents and in combination for 24 h, as determined by Cell Titer Glo. (FIG. 19K) Determining the drug: drug interaction of PP242 with 3 kinase inhibitors, including TGR-1202, idelalisib, and PF4800567. Two methods were used to measure the degree of synergy: relative risk ratio (RRR) and excess over Bliss (EOB). Synergy is reflected by RRR values below 1 or EOB value above 0.

FIG. 20. Co-targeting of CK1ε, PI3Kδ, and the proteasome potently inhibits translation of c-Myc in blood cancers. (FIGS. 20A & B) Western blot analysis of LY10 (A) and LY7 (B) cell lines treated for 24 h by the indicated drugs and concentrations alone and in combination. TG: TGR-1202, Ide: idelalisib, Bz: bortezomib, Cfz: carfilzomib, IB: combination of Ide and Bz, TC: combination of TG and Cfz. (FIG. 20C) mRNA and protein levels of c-Myc in LY10 cells treated as in (A). In addition, TB: TG and Bz, IC: Ide and Cfz. (FIG. 20D) Cap-dependent translation downstream of the c-Myc 5′UTR in LY7 cells treated as indicated. LY7 cells transiently transfected with the bicistronic reporter from FIG. 3C were treated for 24 hr. The R/F Luciferase ratio reflects Myc cap-dependent translation. (FIGS. 20E & F) Effects of eIF4E overexpression on c-Myc protein levels and cell viability in TG+CFZ treated cells. The Myeloma cell line H929 was stably transduced with an eIF4E-overexpressing plasmid (eIF4E) by lentiviral transduction, or with the corresponding empty vector (EV). These cells and the untransduced control (No TDX) cells were treated for 24 hr and assessed by Western blot (E) and Cell-Titer Glo (F). (FIGS. 20G & H) Effects of 5′-UTR null Myc expression on c-Myc protein levels and cell viability in LY7 cells treated by the TG+CFZ combination. LY7 Cells expressing the Myc (Myc) or empty vector (EV) were treated with TC#1 (TG 3 μM and Cfz 5 nM) or TC#2 (TG 5 μM and Cfz 5 nM) for 24 hr, and were assessed by Western blot (G) and Cell-Titer Glo (H). (FIG. 20I & J) Effects of CK1ε knockdown on the combination of Ide+Cfz. LY7 cells stably expressing shRNA targeting CK1ε (CSNK1E kd+) or the parental untransduced control cells (kd−) were treated as indicated for 24 h and assessed by Western blot (I) and Cell-Titer Glo (J).

FIG. 21. Co-targeting of CK1e, PI3Kd, and the proteasome potently inhibits translation of c-Myc in blood cancers. (FIG. 21A) LY7 and LY10 cells were treated by TG (TGR-1202), Ide (idelalisb), Cfz (carfilzomib), Bz (bortezomib) at the indicated mM (for TG and Ide) or nM (Bz and Cfz) concentrations, and the indicated combinations for 24 h. The viability was measured by Cell Titer Glo. RRR and EOB values were calculated as in FIG. 19. Synergy was assessed by two methods including relative risk ratio (RRR) and excess over bliss (EOB) values. RRR<1 indicates synergy. EOB>0 also indicates synergy. (FIG. 21B) The indicated cell lines or primary lymphoma and leukemia cells were treated as indicated and processed for Western blot analysis. TG: TGR-1202, Ide: idelalisib, Bz: bortezomib, Cfz: carfilzomib, IB: combination of Ide and Bz, TC: combination of TG and Cfz. (FIG. 21C) mRNA level of c-Myc in LY7 cells treated with 2 combinations, including (1) Cal (Cal-101/idelalalisib 3 mM) and Bort (bortezomib 5 nM), and (2) TG (TGR-1202 3 mM) and Cfz (carfilzomib 5 nM). (FIG. 21D-E) LY10 (D) and PF382 (E) cells were treated as indicated for 24 h and processed for Western blot.

FIG. 22 Relates to Table A showing results of kinome study of TGR-1202.

### DETAILED DESCRIPTION

### 1. Introduction

It has been discovered that combining select proteasome and PI3K inhibitors is useful for treating c-Myc-overexpressing cancers, particularly hematological cancers such as aggressive B- and T-cell lymphomas. Specifically, it was found that co-administration of the PI3Kδ inhibitor TGR-1202 and the proteasome inhibitor carfilzomib significantly increased cell death of aggressive B- and T-cell lymphomas as well as multiple myeloma over the individual or additive effect of either or both agents. As will be further explained, this synergistic effect is associated with the previously unknown inhibition of the kinase casein kinase 1 epsilon (CK-1ε) by TGR-1202. Accordingly, use of PI3K inhibitors that possess CK-1ε inhibition in combination with proteasome inhibitors provide a new therapy regime for treating c-Myc-overexpressing cancers, and particularly hematological cancers. Alternatively, a PI3K inhibitor and proteasome inhibitor can be combined with a separate CK-1 (typically CK-1ε) inhibitor that is not a dual PI3K/CK-1 inhibitor to realize the synergistic effects observed with a dual PI3K/CK-1 inhibitor and proteasome inhibitor combination. In other embodiments, proteasome inhibitors can be combined with select PI3K inhibitors that have dual function of inhibiting other CK-1 isoforms such as alpha and delta.

Other embodiments relate to the inhibition of CK-1 (e.g. CK1ε) via administration of a CK-1 inhibitor alone. The targeting of CK-1 by a CK-1 inhibitor results in a reduction of c-Myc production in c-Myc overexpressing cancer cells. This treatment therefore modulates the disease state of the c-Myc overexpressing cancer making it less malignant and more susceptible to adjunctive cancer therapies.

Other embodiments relate to a novel class of CK-1 inhibitors, which can be used for cancer therapy or for non-cancer related therapies that involve CK-1. For example, in certain embodiments, CK-1 inhibitors are used to treat autoimmune related diseases or graft versus host disease (GVHD).

Certain embodiments of the invention pertain to a combination therapy for treating c-Myc-overexpressing cancers by the co-administration of dual PI3K/CK-1 inhibitors (e.g. PI3K/CK-1ε inhibitor) with proteasome inhibitors and to pharmaceutical formulations containing both of these inhibitors. Other embodiments involve screening of PI3K inhibitors to identify those that additionally inhibit CK-1ε or other CK-1 isoforms such as alpha or delta isoforms.

In further analyzing the structure of TGR-1202, and comparing its structure to the known CK-1 inhibitor PF4800567, specific structures have been identified as being responsible for the CK-1 inhibition by TGR-1202. This knowledge has enabled the design of a new class of chemical compounds that possess CK-1 inhibitory effects. Accordingly, certain embodiments are directed to these chemical compounds, as well as using these compounds to inhibit CK-1 for treatment of cancer, typically c-myc related cancer, and for treatment of autoimmune-related disorders.

### 2. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention generally are performed according to conventional methods well known in the art and as described in various general and more specific references, unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

As used herein and in the appended claims, the singular forms “a,” “an,” and “the” include plural references unless the content clearly dictates otherwise.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, an “adjunct cancer therapeutic agent” pertains to an agent that possesses selectively cytotoxic or cytostatic effects to cancer cells over normal cells. Adjunct cancer therapeutic agents may be co-administered with a CK-1 inhibitor, dual PI3K/CK-1 inhibitor or a combination of a PI3K-AKT-mTOR signaling pathway inhibitor and CK-1 inhibitor, optionally with a proteasome inhibitor. A non-limiting list of examples of adjunct cancer therapeutic agents is provided in Table 4.

As used herein, the term “adjunct cancer therapy protocol” refers to a therapy, such as surgery, chemotherapy, radiotherapy, thermotherapy, and laser therapy, and may provide a beneficial effect when administered in conjunction with administration of a CK-1 inhibitor, dual PI3K/CK-1 inhibitor and/or a combination of a PI3K-AKT-mTOR inhibitor and CK-1 inhibitor, and any of the foregoing optionally including a proteasome inhibitor. Such beneficial effects include reducing tumor size, slowing rate of tumor growth, inhibiting metastasis, or otherwise improving overall clinical condition, without necessarily eradicating the cancer. Cytostatic and cytotoxic agents that target the cancer cells are specifically contemplated for combination therapy. Likewise, agents that target angiogenesis or lymphangiogenesis are specifically contemplated for combination therapy.

The term “administering” an agent as used herein means providing the agent to a subject using any of the various methods or delivery systems for administering agents or pharmaceutical compositions known to those skilled in the art.

The term “AKT inhibitor” as used herein refers to agents that block or reduce expression or activity of AKT. A non-limiting list of AKT inhibitor examples is provided in Table 1.

The term “co-administration” or “co-administering” as used herein refers to the administration of an active agent before, concurrently, or after the administration of another active agent such that the biological effects of either agents overlap. The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects.

The term “cancer” or “tumor” as used herein means is intended to include any neoplastic growth in a patient, including an initial tumor and any metastases. The cancer can be of the liquid or solid tumor type. Liquid tumors include tumors of hematological origin (hematological cancer), including, e.g., myelomas (e.g., multiple myeloma), leukemias (e.g., Waldenstrom's syndrome, chronic lymphocytic leukemia, other leukemias), and lymphomas (e g, B-cell lymphomas, non-Hodgkins lymphoma). Solid tumors can originate in organs, and include cancers such as lung, breast, prostate, ovary, colon, kidney, and liver. In a specific embodiment, cancer pertains to c-Myc-overexpressing cancer.

The term “cancerous cell” or “cancer cell” as used herein means a cell that shows aberrant cell growth, such as increased cell growth. A cancerous cell may be a hyperplastic cell, a cell that shows a lack of contact inhibition of growth in vitro, a tumor cell that is incapable of metastasis in vivo, or a metastatic cell that is capable of metastasis in vivo. Cancer cells include, but are not limited to, carcinomas, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large B cell lymphoma, malignant lymphoma, plasmocytoma, reticulum cell sarcoma, or Hodgkins disease).

The terms, “cancerous B cell” and “cell of a B cell cancer” are used interchangeably herein to refer to a B cell that is cancerous.

The term “casein kinase 1” or “CK-1” refers to an enzyme pertaining to the casein kinase 1 protein kinase family, or an active fragment or active variant thereof having at least 90% identity thereto. The casein kinase 1 family is evolutionarily conserved with seven mammalian isoforms: α, β, γ1, γ2, γ3, δ, and ε. Set forth below in Table 2 is the human amino acid sequence for CK-1ε (SEQ ID No. 1) and the related mRNA sequence (SEQ ID NO. 2). CK-1ε is known to regulate circadian rhythms by phophorylating other clock proteins, such as PERIOD. Over expression of CK-1ε mimics WNT-signaling through phosphorylation of Tcf3 and stabilization of β-catenin, suggesting a functional role in stem cell properties.

The term “CK-1 inhibitor” as used herein refers to agents that block or reduce expression or activity of CK-1. Examples of CK-1 inhibitors are provided in Table 3, or analogs, derivatives or pharmaceutically acceptable salts thereof.

The term “CK-1 reducing effective amount” as used herein means an amount of a CK-1 inhibitor administered to a subject that reduces activity of CK-1 in the subject by at least 30, 40, 50 or 60 percent of its normal activity.

The term “c-Myc” as used herein means the transcription factor encoded by the proto-oncogene c-myc that controls cell proliferation. c-Myc also plays a role in regulating cell cycle, cell growth, angiogenesis, apoptosis, and oncogenesis. The c-Myc transcription factor is of the helix-loop-helix leucine zipper class and plays a role in the modulation and initiation of transcription. c-Myc binds to E-boxes (CACGTG) in the vicinity of target genes, which are then activated. The DNA binding activity requires dimerization with another helix-loop-helix leucine zipper protein called Max. Max can also interact with transcriptional repressors such as Mad and Mxil, which presumably down-regulate expression of c-Myc target genes. c-Myc, when activated, can induce malignancy in a variety of tissues, most notably hematopoietic tissues (Leder et al., 222 Science 765, 1983). Myc's activity can increase in tumors as a consequence of mutations, chromosomal rearrangements, increased expression, or gene amplification, elevated or deregulated expression of c-Myc has been detected in a wide range of human cancers and is often associated with aggressive, poorly differentiated tumors. Such cancers include colon, breast, cervical, small cell lung carcinomas, osteosarcomas, glioblastomas, melanoma and myeloid leukemias.

The term “c-Myc-overexpressing cancer” as used herein relates to any cancer wherein the cancer cells overexpress c-Myc as compared to normal, healthy cells. Overexpression of c-Myc includes elevated RNA transcript or protein levels of c-Myc as compared to healthy, normal cells. c-Myc-overexpressing cancers comprise hematological cancers such as, myelomas (e.g. multiple myeloma), leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B cell lymphoma, malignant lymphoma, plasmocytoma, reticulum cell sarcoma, or Hodgkins disease) and solid-tumor cancers of the lung, breast, prostate, ovary, colon, kidney, and liver.

The term “c-Myc reducing effective amount” as used herein means an amount of an enumerated agent administered to a subject that reduces a level of c-Myc in cells of a subject.

The term “dual PI3K/CK-1 inhibitor” as used herein includes agents that reduce the biological activity or expression of both PI3K and one or more isoforms of CK-1. Typically, a dual PI3K/CK-1 inhibitor reduces activity of PI3Kδ and CK-1α, δ and/or ε. Dual inhibition of PI3K with CK1 ε has been shown to have a strong synergistic effect of killing cancer cells in conjunction with proteasome inhibitor administration.

The term “enumerated therapeutic agent(s)” or “enumerated agents” as used herein refers to any of a PI3K-AKT-mTOR signaling pathway inhibitor, proteasome inhibitor, CK-1 inhibitor or adjunct cancer therapeutic agent. Enumerated therapeutic agents may include analogs, derivatives or pharmaceutically acceptable salts of any agent specified herein.

The term “enumerated disease” as used herein refers to any cancer or other disease described herein as being treatable using embodiments of the invention, more specifically it includes myelomas (e.g. multiple myeloma), leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large B cell lymphoma, malignant lymphoma, plasmocytoma, reticulum cell sarcoma, or Hodgkins disease). Enumerated disease may also include organ rejection in transplant patients, graft versus host disease (GVHD), and autoimmune diseases, including rheumatoid arthritis, psoriasis, eczema, asthma, multiple sclerosis, inflammatory bowel disease, Crohn's disease, colitis (e.g., ulcerative colitis), systemic lupus erythematosus, myasthenia gravis, Sjögren's syndrome and sclerodema, autoimmune hemolytic anemia, cold agglutinin disease, and IgA nephropathy.

The terms “hematological cancer” or “hematological malignancies” are used interchangeably and pertain to malignant neoplasms that derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

The term “mTOR inhibitor” as used herein refers to agents that block or reduce expression or activity of mTOR. A non-limiting list of mTOR inhibitor examples is provided in Table 1.

The term “n-Myc” as used herein means the n-Myc proto-oncogene protein that is a protein encoded by the MYCN gene. The terms n-Myc, MYCN or NMYC are used interchangeably herein. The gene is a member of the MYC family of transcription factors. The expressed protein contains a basic helix-loop-helix domain and must dimerize with another basic helix-loop-helix domain to bind DNA. Like c-Myc, the MYCN protein interacts with MAX. Amplification of the MYCN gene is mostly associated with a variety of tumors, most notably neuroblastomas.

The term “phosphoinositide 3-kinase (PI3K) inhibitor(s)” as used herein includes agents that block or reduce expression or activity of PI3K. Examples of PI3K inhibitors are provided in Table 1. PI3K inhibitors for use in embodiments of the invention are also described in U.S. Pat. Nos. 8,642,607; 8,912,331; and 9,018,375. In a specific embodiment, the PI3K inhibitor inhibits PI3Kδ.

The term “PI3K-AKT-mTOR signaling pathway inhibitor” refers to any of a PI3K inhibitor, dual PI3K/CK-1 inhibitor, AKT inhibitor, or mTOR inhibitor. A non-limiting list of examples of these inhibitors is provided in Table 1.

The term “proteasome(s)” as used herein refers to protein complexes inside eukaryotes that are located in the nucleus and the cytoplasm that function to degrade unneeded or damaged proteins by proteolysis. Proteasomes are abundant multi-enzyme complexes that provide the main pathway for degradation of intracellular proteins and contribute to the maintenance of protein homeostasis and clearance of misfolded and/or unfolded and cytotoxic proteins. The ubiquitin-proteasome pathway (UBP) modulates intracellular protein degradation. Specifically, the 26S proteasome is a multi-enzyme protease that degrades misfolded or redundant proteins; conversely, blockade of the proteasomal degradation pathways results in accumulation of unwanted proteins and cell death. Because cancer cells are more highly proliferative than normal cells, their rate of protein translation and degradation is also higher. Thus, cancer cells are more dependent on the proteasome for clearance of abnormal or mutant proteins than normal cells.

The term “proteasome inhibitor(s)” as used herein pertains to an agent(s) that blocks or reduces the action of proteasomes. Examples of proteasome inhibitors are provided in the Therapeutic Agents section provided below.

The terms “subject,” “individual,” “host,” and “patient,” are used interchangeably herein to refer to an animal being treated with one or more enumerated agents as taught herein, including, but not limited to, simians, humans, avians, felines, canines, equines, rodents, bovines, porcines, ovines, caprines, mammalian farm animals, mammalian sport animals, and mammalian pets. A suitable subject for the invention can be any animal, preferably a human, that is suspected of having, has been diagnosed as having, or is at risk of developing a disease that can be ameliorated, treated or prevented by administraton of one or more enumerated agents.

The term “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a disease, disorder, or condition or one or more symptoms or manifestations of a disease, disorder or condition.

A “therapeutically effective amount” refers to an amount which, when administered in a proper dosing regimen, is sufficient to reduce or ameliorate the severity, duration, or progression of the disorder being treated (e.g., cancer, GVHD, or autoimmune disease), prevent the advancement of the disorder being treated (e.g., cancer, GVHD, or autoimmune disease), cause the regression of the disorder being treated (e.g., cancer, GVHD, or autoimmune disease), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

### 3. Overview

The results described in the Examples show that TGR-1202, a PI3Kδ inhibitor with promising clinical activity and an excellent safety profile, and carfilzomib, an FDA approved proteasome inhibitor, are highly synergistic in causing cancer cell death in cell line models of hematological malignancies and primary tumor cells. Other drugs with comparable single-agent activity of the same class appear to demonstrate substantially less to no synergy in the same model systems. The marked synergism of TGR-1202 and carfilzomib in lymphoma was associated with the unexpected ability of this combination to potently inhibit mTOR, the phosphorylation of 4EBP1, translation of c-Myc, and certain downstream functions of c-Myc; and TGR-1202 was also found to be a potent inhibitor of CK-1 epsilon whereas other PI3K inhibitors did not have this inhibitory action and were less effective in killing cancer in cancer cell lines. The combination of TGR-1202 and carfilzomib was also shown to have an unexpected ability to synergistically kill multiple myeloma cell lines.

Therefore, certain embodiments are directed to methods of treating c-Myc-overexpressing cancers, including aggressive lymphoma, by combining select proteasome and PI3K inhibitors, including the embodiment wherein the PI3K inhibitor has CK-1 epsilon inhibitory activity. Thus, the ‘right’ combination for clinical development may be chosen rationally among various combinations of PI3K and proteasome inhibitors based on their optimal ability to disrupt the mTOR-eIF4F-c-Myc axis in preclinical studies thereby decreasing c-Myc expression. Inhibitors having dual activity against PI3K and CK-1 are particularly useful in combination therapies with proteasome inhibitors. In view of this discovery, pharmaceutical formulations can be made that include: 1) generally a PI3K-AKT-mTOR signaling pathway inhibitor and a CK-1 inhibitor; 2) a dual PI3K/CK-1 inhibitor and proteasome inhibitor; 3) PI3K inhibitor, CK-1 inhibitor, and proteasome inhibitor; 4) dual PI3K/CK-1 inhibitor, separate CK-1 inhibitor and proteasome inhibitor; or 5) a CK-1 inhibitor in combination with a PI3K inhibitor or a proteasome inhibitor. Generally, the above formulations can be used for treating c-Myc overexpressing cancers. These formulations may optionally include adjunct cancer therapeutic agents.

c-Myc is a master transcription factor and one of the most frequently altered genes across a vast array of human cancers [1]. Overexpression of c-Myc is observed in up to 30% of cases of diffuse large B-cell lymphoma (DLBCL) [2], the most common type of aggressive lymphoma. Although DLBCL can be cured in 60-70% patients [3], a substantial minority of patients with DLBCL still die from their lymphoma. DLBCL can be divided by gene-expression profiling (GEP) studies into germinal center B cell-like (GCB) and activated B cell-like (ABC) subtypes. While the ABC subtype has an inferior prognosis compared to the GCB subtype [4], there is emerging evidence that c-Myc overexpression is an independent risk factor in both subtypes.

The most common mechanism of c-myc activation is translocation to any of the immunoglobulin (Ig) or T cell receptor loci during lymphoid maturation. For example, in Burkitt's lymphoma the c-myc locus on chromosome 8 translocates most often to the Ig heavy chain locus on chromosome 14, but also to the lambda or kappa light chain Ig genes on chromosomes 2 and 22 (Magrath, in “Epstein-Barr Virus and Associated Diseases”, M. Nijhoff Publishing: 631, 1986). In some instances the c-myc transcription region is altered in the non-coding exon 1 region; in such cases transcription is initiated at a cryptic promoter present in the first intron of the c-Myc locus.

Early studies by Savage [5] and Barrans [6] reported that 9 to 14% patients with newly diagnosed DLBCL harbor a c-Myc gene rearrangement. In patients treated with the regimen R-CHOP (Rituximab, Cyclophosphamide, Hydroxydaunorubicin (doxorubicin), Oncovin (vincristine), and Prednisone), c-Myc gene rearrangement was associated with an inferior overall survival that is only half of that for patients without c-Myc translocation. Subsequently Johnson [7], Green [8], and Hu [9] independently observed a similar frequency of c-Myc rearrangement (10-15%) and a significantly higher frequency (30%) of c-Myc protein expression in large sample sets of newly diagnosed DLBCL patients. Further, these later studies demonstrated that the poor survival associated with dysregulated c-Myc was present only when another oncogene, Bcl2, is also overexpressed. DLBCL with co-expression of the c-Myc and Bcl-2 proteins, i.e. double positive (DP)-DLBCL, is significantly enriched with the ABC than the GCB subtype, and appears to be the primary cause of the relatively poor survival of the ABC subtype. “Double hit” lymphoma (DHL) represents 5% of all DLBCL [10], and is characterized by chromosome rearrangements involving both c-Myc and Bcl2. DLBCL exhibits an even worse prognosis than DP-DLBCL [11], and intriguingly, demonstrates an immunohistochemical staining consistent with the GCB subtype in most cases [12].

The c-Myc protein has a short half-life, less than 30 minutes [13], and needs to be produced constantly in c-Myc driven cancers. The complex secondary structure of the 5′ untranslated region (UTR) of c-Myc makes its translation highly dependent on the eukaryotic initiation factor 4F (eIF4F) [14, 15]. eIF4F exists as a complex comprised of eIF4E, eIF4A, and eIF4G. eIF4E is the rate limiting factor for eIF4F, as eIF4E can be sequestered by 4EBP1 [16]. The mammalian target of rapamycin (mTOR) causes phosphorylation dependent inactivation of 4EBP1, leading to release of eIF4E from 4EBP1 and assembly of the eIF4F complex. mTOR is activated through the PI3K-AKT pathway. Furthermore, the ubiquitin-proteasome system is also critically involved in the activation of mTOR [17-19]. Conversely, activated mTOR can increase the levels of intact and active proteasomes through a global increase in the expression of genes encoding proteasome subunits [20].

c-Myc itself can act as an upstream stimulator of mTOR [21], and is required for the transcription of the eIF4F subunits [14]. It has been observed that mTOR acts as a nexus that coordinates complex upstream signals to stimulate eIF4F dependent translation of c-Myc. Without being bound by theory, it is believed that if the proteasome and PI3K pathways cooperate in the activation of mTOR and its downstream target eIF4F, then combinations of drugs targeting the proteasome and PI3K will be able to potently downregulate eIF4F dependent translation of c-Myc, leading to synergistic inhibition or death of c-Myc dependent lymphoma. Furthermore, since both the PI3K and proteasome pathways are validated drug targets in hematological malignancies, a strategy that combines PI3K and proteasome inhibition has the potential to rapidly advance to clinical studies and benefit patients.

For many years c-Myc has been the prototypical example of an “undruggable” oncogene. Increased understanding of the role of bromodomains in mediating protein-protein interaction on the chromatin has created new opportunities in down-regulating transcriptional activators [30-36].

A few BRD4 inhibitors have entered small phase I clinical studies, but the safety and toxicity data are not available yet. Seminal work by Pelletier's group discovered an eIF4F-Myc feed-forward loop whereby eIF4F stimulates the translation of c-Myc, and c-Myc enhances the transcription of eIF4F subunits [14]. The inventors have realized that the inter-dependence of eIF4F and c-Myc creates another opportunity to inhibit the oncoprotein. Recently, a number of small molecule inhibitors have been identified that inhibit either the activity or interaction of eIF4F subunits and cofactors, leading to down-regulation of c-Myc, direct killing of cancer cells, and enhanced sensitivity of cancer cells to chemotherapeutic agents in cell line and animal models [14, 15, 37-42]. None of these eIF4F inhibitors have entered clinical development.

An alternative approach has been used to inhibit c-Myc by exploiting agents already well-established as safe and active in the clinic, by targeting upstream cancer specific signals that converge on mTOR. For example, PI3K-AKT is a well-established activator of mTOR and a proven target for cancer treatment judging from the recent approval of idelalisib, a PI3Kδ inhibitor for the treatment of chronic lymphocytic leukemia. Similarly, the proteasome pathway is involved in the activation of mTOR, and has been successfully targeted for cancer treatment. However, until now no synergy has ever been demonstrated by combining certain PI3K and proteasome inhibitors to treat cancers such as hematomological cancer. As demonstrated below, not all PI3K inhibitors possess this synergy with all proteasome inhibitors. For example, idelalisib possessed negligible synergy with carfilzomib in killing lymphoma or multiple myeloma cells.

### 4. Summary of the Results

The following is a summary of results of experiments described in the Examples of this application.
TGR-1202 is a novel PI3Kδ inhibitor whose activity and isoform selectivity are comparable to idelalisib.
TGR-1202 and carfilzomib demonstrated superior activity and synergy among four combination pairs of PI3K and proteasome inhibitors in DLBCL.
TGR-1202 and carfilzomib were consistently the most synergistic pair among four combinations of PI3K and proteasome inhibitors in aggressive B cell and T cell lymphomas and multiple myeloma.
TGR-1202 and carfilzomib in combination markedly inhibited signaling in the mTOR-eIF4F-Myc axis in models of B- and T-cell lymphoma.
TGR-1202 and carfilzomib in combination potently inhibited the cap dependent translation of c-Myc.
TGR-1202 and carfilzomib in combination were highly active against primary lymphoma cells but not toxic to normal lymphocytes.
TGR-1202 possesses dual inhibition activity: PI3K and CK-1ε inhibition.
CK1ε inhibition is synergistic with carfilzomib in lymphoma, producing effective suppression of c-Myc.
PI3K inhibition alone is not synergistic with carfilzomib.
The synergistic effect between TGR-1202 and carfilzomib is directly related to TGR-1202's dual inhibition of PI3K and CK-1ε coupled with proteasome inhibition of carfilzomib.
A triad combination of a PI3K inhibitor (other than TGR-1202), a CK-1 inhibitor and carfilzomib replicates the effects of TGR-1202 and carfilzomib.
Based on x-ray crystal structure analysis, TGR-1202 is structurally related to the known CK-1ε inhibitor PF4800567. In silico docking studies targeting the ATP binding pocket of CK1ε showed that TGR-1202 possessed high docking scores in binding modes highly consistent with PF4800567.
Idelalisib possessed important steric clashes and low docking scores for the ATP binding pocket of CK1ε.
Newly synthesized compound CUX-03173 possessed a top-binding pose very close to that of TGR-1202 and a similar docking score with respect to the ATP binding pocket of CK1ε.
TGR-1202 was active against CK1ε, with an IC50 value of 6.0 μM. The IC50 for

CUX-03173 was 9.4 μM.

TGR-1202+Carfilzomib have Synergistic Effects in Treating Hematologic Cancer

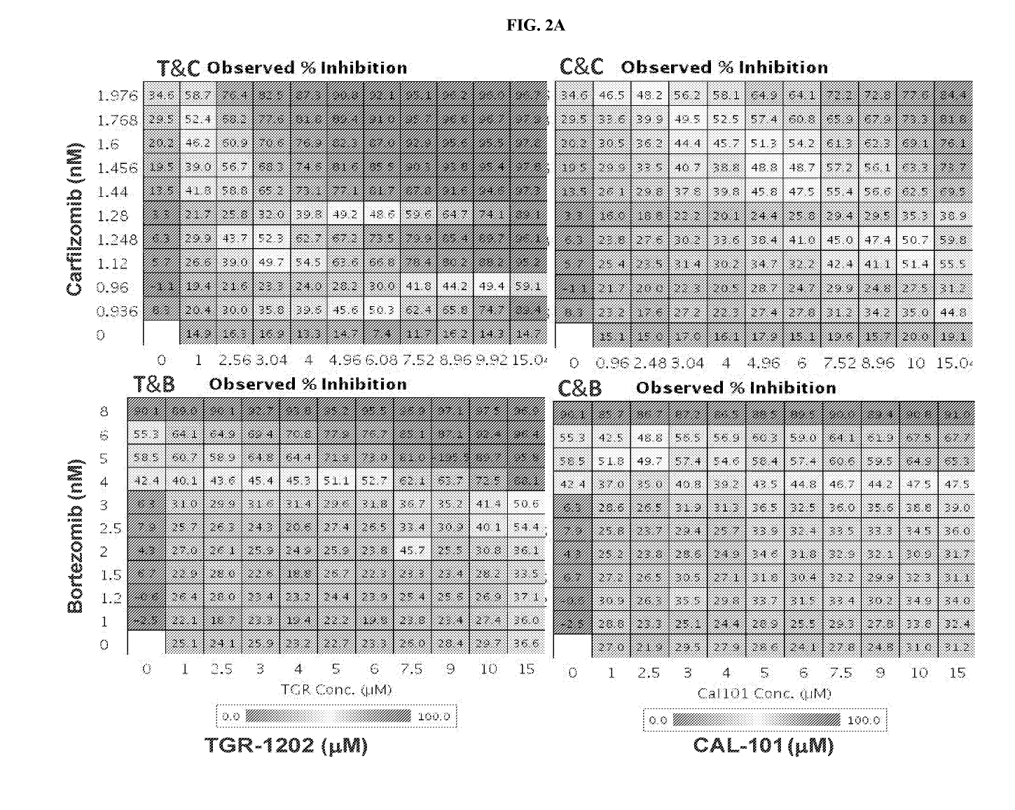

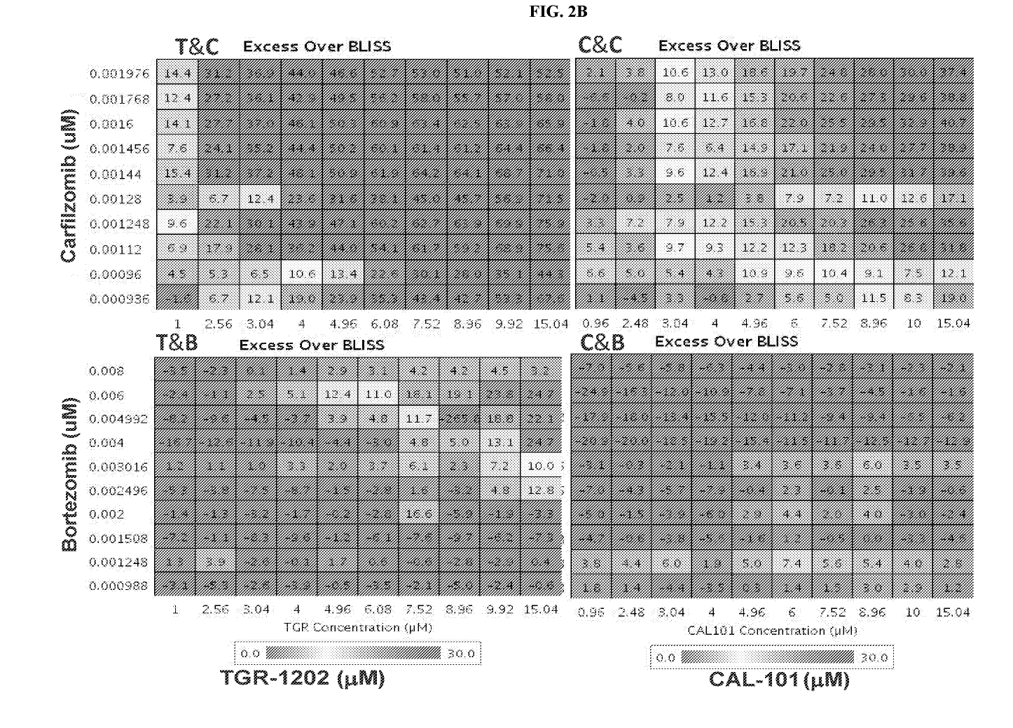

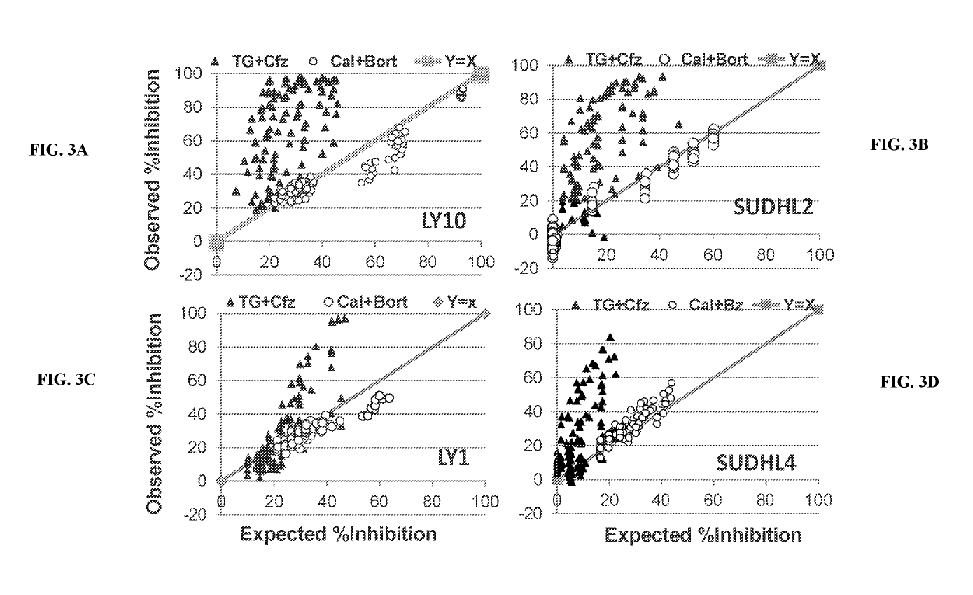

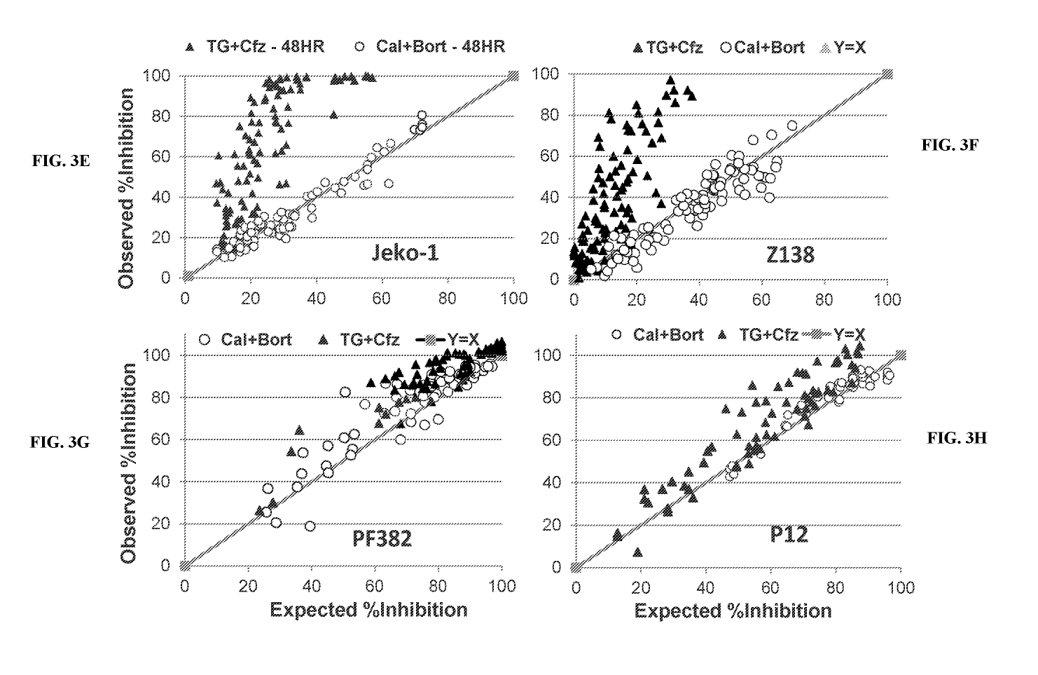

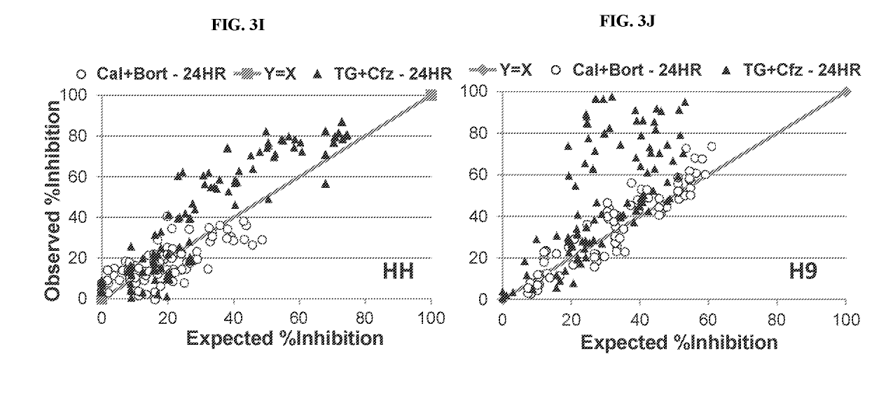

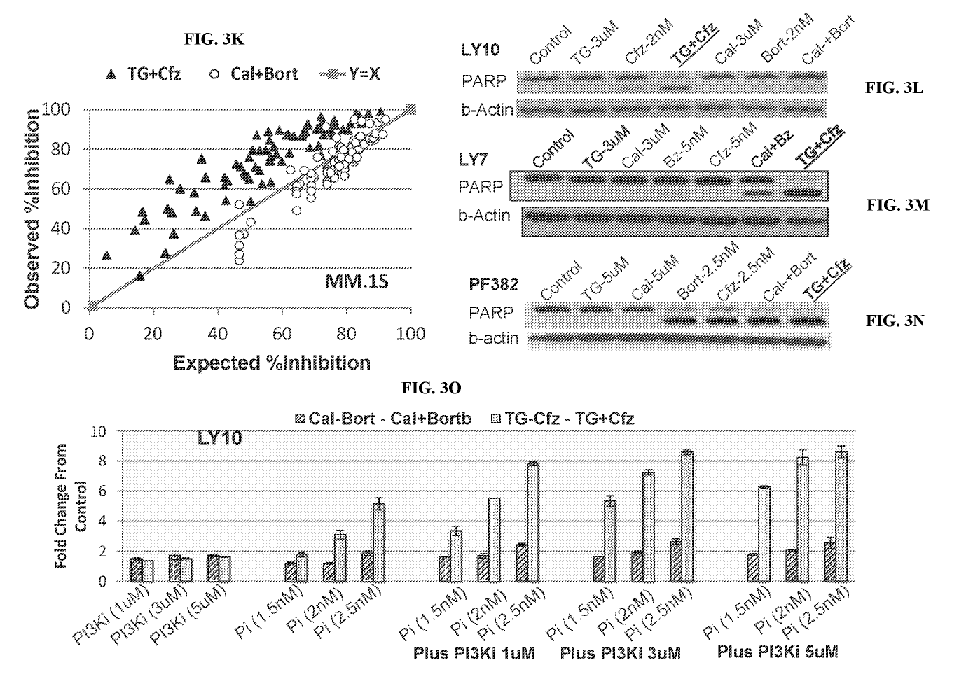

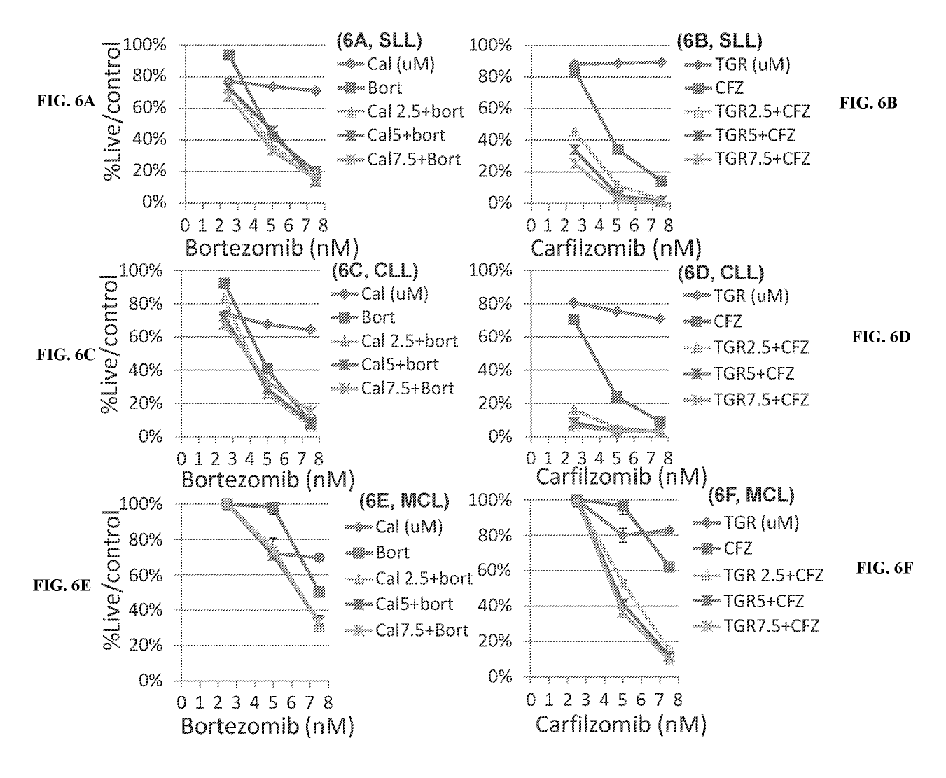

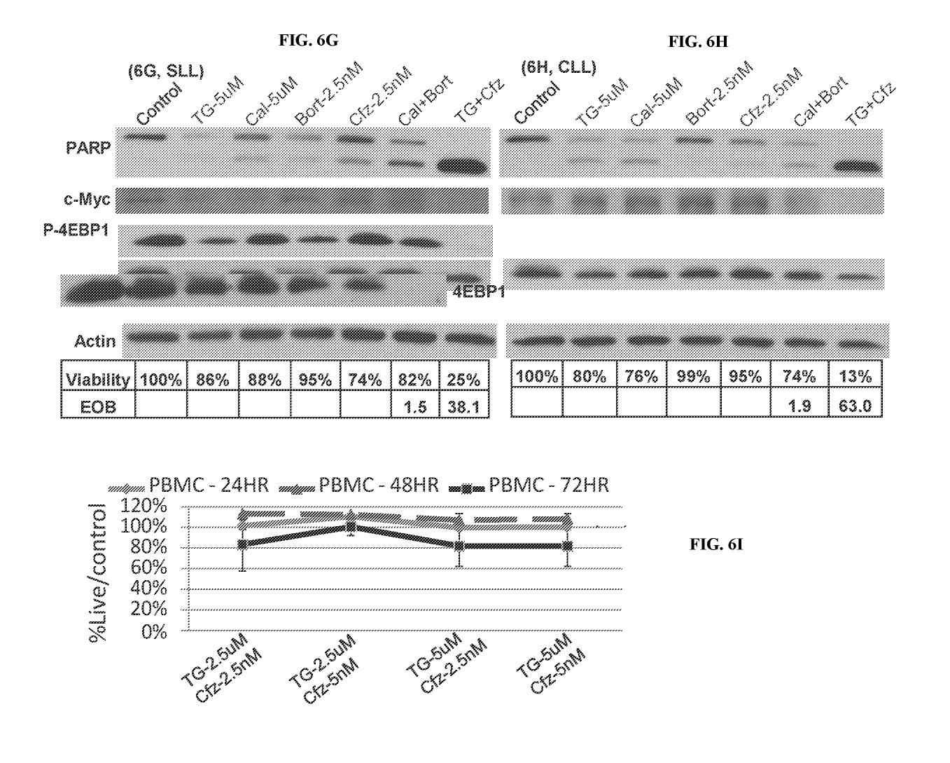

It has now been discovered that certain PI3K inhibitors were highly synergistic with certain proteasome inhibitors. Furthermore, the inhibition of tumor growth and the induction of apoptosis in a broad panel of lymphoma cell lines and primary tumor tissues was surprisingly restricted to certain unique combinations (FIG. 2, FIG. 3, & FIG. 6), while other combination pairs were much less or not at all synergistic. Specifically, TGR-1202 in combination with carfilzomib showed an extraordinarily high level of synergy, whereas Cal-101 and carfilzomib showed little synergy.

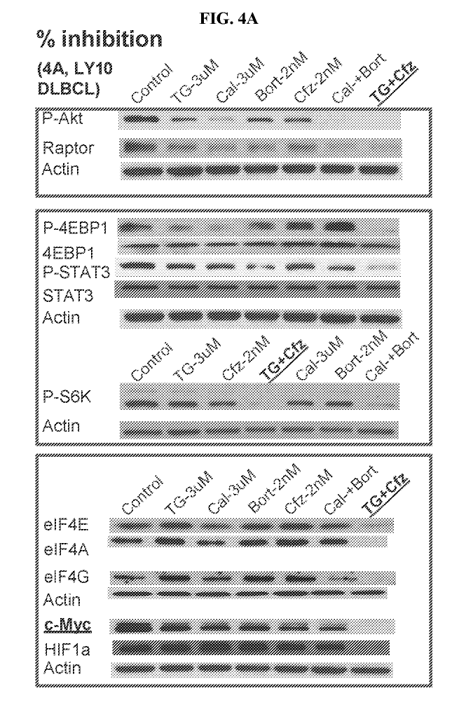

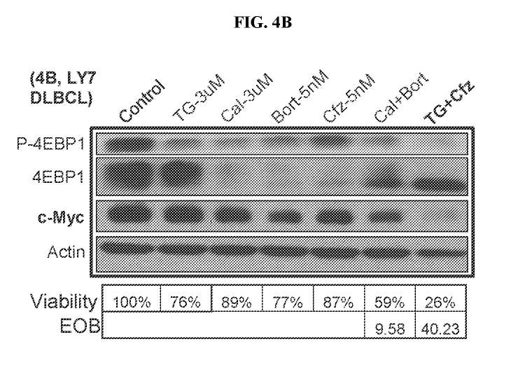

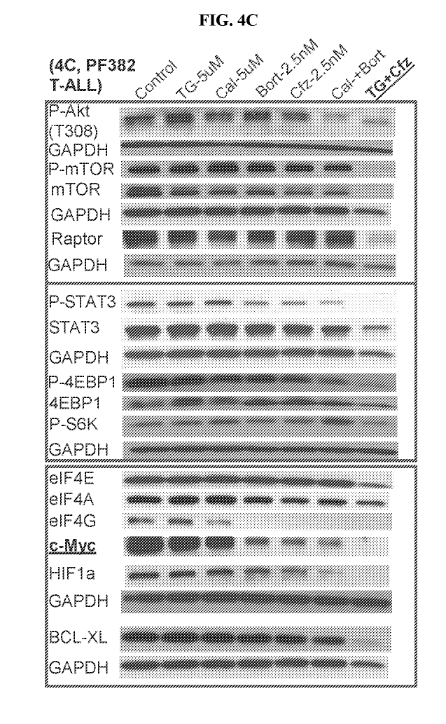

To elucidate why only select combinations of PI3Kδ inhibitors and proteasome inhibitors are synergistic in lymphoma, the PI3K-AKT-mTOR-eIF4F-Myc signal cascade in LY10 lymphoma cells was treated by administering different PI3Kδ inhibitors and proteasome inhibitors, either as single agents or in combinations. The results showed that the PI3Kδ inhibitors TGR-1202 and idelalisib had mild to moderate inhibition of AKT phosphorylation at pharmacologically available concentrations of 3 μM and idelalisib was generally more potent than TGR1202 (FIG. 4). These PI3Kδ inhibitors produced minimal or no inhibition of 4EBP1 phosphorylation or the protein level of c-Myc expression (FIG. 4-FIG. 6). Similarly, the proteasome inhibitors carfilzomib and bortezomib had minimal or no effect on AKT and 4EBP1 phosphorylation at low concentrations of about 2 nM (FIG. 4), and they produced only a mild to moderate reduction in the protein level of c-Myc (FIG. 4-FIG. 6).

By contrast, the TGR-1202+carfilzomib and Cal-101+bortezomib combinations were both associated with potent inhibition of AKT phosphorylation. Remarkably, the combination pair TGR-1202+carfilzomib was much more effective than either single agent or any of the other combination pairs tested on inhibiting the phosphorylation of mTOR and 4EBP1 and reducing the protein level of c-Myc (FIG. 4-FIG. 6). These results showed that different combination pairs of PI3Kδ and proteasome inhibitors produced divergent biologic effects at the nexus of mTOR, with the combination of TGR-1202 and carfilzomib being most potent at inhibiting mTOR. Without being bound by theory, these two compounds, more than any other combination of PI3δ and proteasome inhibitors that were tested had a synergistic inhibitory effect on a regulatory protein that undergoes phosphorylation and degradation.

One logical candidate for this regulatory protein is DEPTOR, a negative regulator of mTOR that undergoes phosphorylation-dependent degradation by the E3 ligase βTrCP [43-45]. However, the protein level of DEPTOR was potently suppressed by the TGR-1202/carfilzomib combination, and was unaffected by any other single agents or combinations (data not shown). Thus DEPTOR cannot account for the unique synergy of TGR-1202 and carfilzomib. Instead it was discovered that it was the dual inhibition of both PI2K and CK-1ε by TGR-1202 that caused the superior inhibition of mTOR seen with the TGR-1202/carfilzomib combination. It is also possible that the differential effects on mTOR by various combination pairs is a consequence, rather than the cause, of the downregulation of c-Myc, as Myc itself has been shown to regulate the transcription of mTOR [18, 19, 46].

TGR-1202 and Carfilzomib in Combination Potently Suppressed Translation of c-Myc in Lymphoma

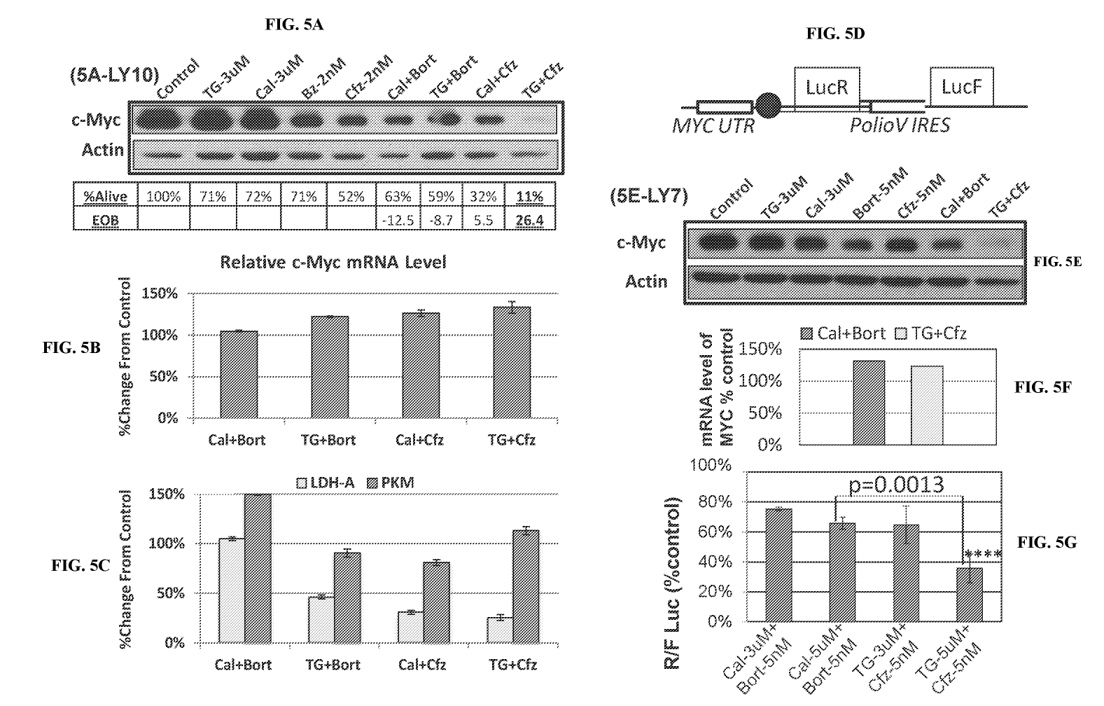

Multiple lines of evidence support discovery that TGR-1202 and carfilzomib in combination potently suppressed translation of c-Myc in lymphoma (FIG. 5). First, the protein level of c-Myc was markedly reduced only by the combination of TGR-1202 and carfilzomib. Secondly, in the presence of proteasome inhibitors, degradation of c-Myc was potently inhibited and therefore could not be the driving force of c-Myc downregulation. Thirdly, the c-Myc mRNA level was not altered by the combination of TGR-1202 and carfilzomib. Lastly, a luciferase reporter assay confirmed that the synergistic combination TGR-1202 and carfilzomib potently inhibited cap-dependent translation downstream of the 5′ UTR of MYC, showing that the decrease in c-Myc levels involves a translational event. Furthermore, reduced expression of c-Myc protein with the combination TGR-1202+carfilzomib was associated with an expected downregulation of Myc target genes such as LDH-A, TK1, TYMS, RPIA, SCN, and upregulation of p21, a gene repressed by c-Myc at the transcription level (FIG. 5C).

The novel PI3Kδ inhibitor, TGR-1202, was potently synergistic with the proteasome inhibitor carfilzomib in broad histological subtypes of lymphoma. The anti-tumor activity of this combination was associated with potent disruption of the mTOR-eIF4F-Myc axis, ultimately leading to deeply suppressed translation of c-Myc protein without affecting the transcription of MYC, and downregulated transcription of Myc target genes. In contrast, other combinations of PI3Kδ and proteasome inhibitors lack synergy and do not disrupt the mTOR-eIF4F-Myc axis. As is discussed below, it was discovered that the specific ability of TGR-1202 to inhibit CK-1ε was a factor in this dramatic synergy.

The widely varied activities of these combinations appear to stem from their divergent effects on mTOR, with TGR-1202/carfilzomib producing the most effective inhibition. However, the mechanism of the anti-tumor activity of TGR-1202 and carfilzomib in combination was likely to involve more than the inhibition of mTOR and the downstream eIF4F-Myc axis. Proteasome inhibitors as a class are pleiotropic drugs, whose best characterized mechanism of action includes activation of the pro-apoptotic responses of the endoplasmic reticulum (ER) stress and unfolded protein response (UPR) pathways.

It is proposed that the TGR-1202/carfilzomib combination disrupts the mTOR-eIF4F-Myc axis, thereby sensitizing cancer cells to the pro-apoptotic actions of carfilzomib. From the clinical perspective, TGR-1202 and carfilzomib are associated with favorable and non-overlapping toxicity profiles [47, 48]. Importantly, the combination was not toxic ex vivo to lymphocytes from healthy donors. Therefore certain embodiments are directed to methods of treating hematologic cancers including aggressive lymphomas and other c-Myc-overexpressing cancers with TGR-1202/carfilzomib combination therapy.

Dual Inhibition of PI3K and CK-1 Contributes to Synergistic Results when Administered with Proteasome Inhibitors

To determine whether the unique effects of TGR-1202 might involve control of phosphorylation via an alternate pathway, it was explored whether TGR-1202 inhibits any other kinases which could account for its unique synergy with carfilzomib. PI3K inhibitors TGR-1202, Cal-101, and IPI-145 were used to test against a battery of different kinases to determine if this compound has any modulating effects, see Table A. As shown in Table A, none of the PI3K inhibitors had any effect on any of the kinases tested except that TGR-1202 had an inhibitory effect on CK-1ε that was not shared by any of the other PI3K inhibitors.

Inhibition of Both PI3K and CK-1ε is Needed to Achieve Synergy with Proteasome Inhibitors

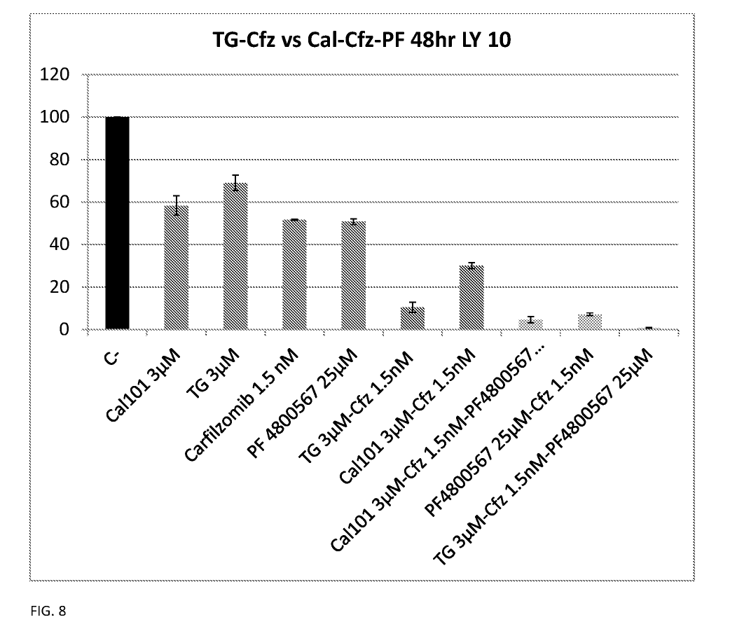

To determine if TGR-1202's unique synergy with carfilzomib was due to its ability to inhibit CK-1ε, an experiment was conducted to test a combination of a different PI3K inhibitor (CAL-101) that has no known CK-1ε inhibitory activity and the known CK-1ε inhibitor (PF-4800567/2) together with the proteasome inhibitor carfilzomib. The rationale was that if CK1 epsilon is involved in promoting lymphoma through regulating the phosphorylation of 4EBP1 and stimulating mRNA translation, then CK1 targeting agents will be synergistic with carfilzomib and other PI3K inhibitors. Indeed, the results in FIG. 8 show that the combination of CAL-101/PF-4800567/2/carfilzomib inhibited c-Myc similar to the TGR-1202/carfilzomib combination, showing that the inhibition of CK-1ε by TGR-1202 was in addition PI3K inhibition was responsible for its unique synergy with carfilzomib.

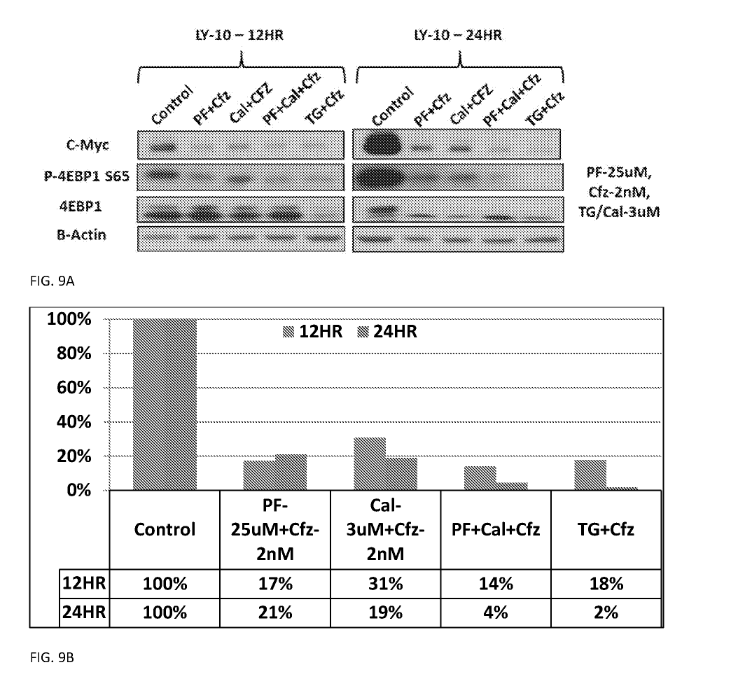

It was determined that CK-1ε inhibition coupled with PI3K inhibition results in a sustained inhibition of 4EBP1 phosphorylation and synthesis of c-Myc. FIG. 9A shows that at 10-12 hours PF-4800567/2 and carfilzomib caused a decrease in c-Myc expression and a decrease in 4EBP1 phosphorylation (represented by P-4EBP1 S65). As shown, the CAL-101/carfilzomib/PF-4800567/2 triple combination and the TGR-1202/carfilzomib double combination also showed a decrease in c-Myc and 4EBP1 phoshorylation at 10 hours. However, at 24 hours c-Myc expression rebounded in the cells treated with the dual PF-4800567/2/carfilzomib combination. In contrast, both the triple CAL-101/carfilzomib/PF-4800567/2 combination and the TGR-1202/carfilzomib combination that include CK-1ε inhibition showed sustained decreases in c-Myc expression and 4EBP1 phosphorylation over longer times, with the TGR-1202/carfilzomib combination showing the greatest decrease. This data shows that both PI3K and CK-1ε inhibition are both needed together with proteasome inhibitors to achieve sustained inhibition of c-Myc synthesis and inhibition of 4EBP1 phosphorylation. (See FIG. 9B.)

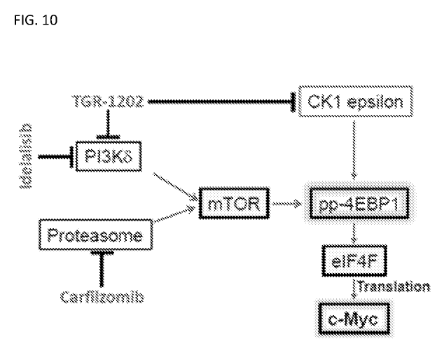

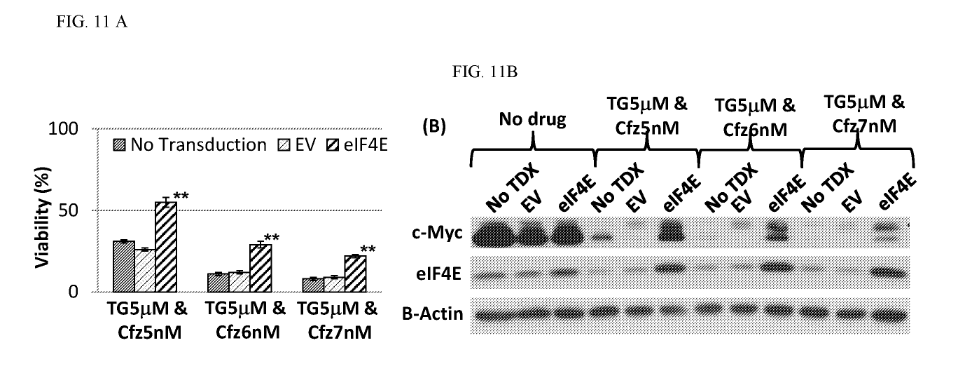

To help illustrate how targeting the PI3K-AKT-mTOR and CK1 pathways may suppress c-Myc, Applicants have developed the model shown in FIG. 10. Without being bound to any theory, it is believed that inhibition of PI3K and CK-1, in combination with proteasome inhibition, serves to inhibit phosphorylation of 4EBP1, which in turn suppresses the mechanisms required for inducing c-Myc synthesis.

### 5. Detailed Description of Embodiments

### Therapeutic Agents

Enumerated agents useful in embodiments of the therapeutic methods described herein for treating c-Myc-overexpressing cancers or hematologic cancers include any of a PI3K-AKT-mTOR signaling pathway inhibitor, proteasome inhibitor, CK-1 inhibitor or adjunct cancer therapeutic agent, including analogs or derivatives thereof, or pharmaceutically acceptable salts thereof. In select embodiments, enumerated agents include PI3K inhibitors, preferably those with dual PI3K and CK-1 inhibitory functions; proteasome inhibitors and inhibitors of various isoforms of CK-1, preferably CK-1ε where hematologic cancers and myeloma are being treated, including analogs or derivatives thereof, or pharmaceutically acceptable salts thereof. Tables 1 and 3 provide specific examples of PI3K inhibitors and CK-1 inhibitors, respectively, contemplated for use as anti-cancer agents. Use of PI3K inhibitors that possess CK-1ε inhibition in combination with proteasome inhibitors provide a new therapy regime for treating c-Myc-overexpressing cancers, and particularly hematological cancers. In other embodiments, proteasome inhibitors can be combined with select PI3K inhibitors that have dual function of inhibiting other CK-1 isoforms such as alpha and delta.

Combinations of inhibitors can be used in co-administration therapy or in preparation of formulations, including the following: 1) a dual PI3K/CK-1 inhibitor and proteasome inhibitor; 2) a PI3K-AKT-mTOR signaling pathway inhibitor inhibitor, CK-1 inhibitor, and proteasome inhibitor; 3) dual PI3K/CK-1 inhibitor, separate CK-1 inhibitor and proteasome inhibitor; 4) a dual PI3K/CK-1 inhibitor and, optionally, an adjunct cancer therapeutic agent (excluding proteasome inhibitor; 5) combination of a PI3K-AKT-mTOR signaling pathway inhibitor (i.e. PI3K inhibitors, AKT inhibitors, and mTOR inhibitors) and a CK-1 inhibitor, and optionally, an adjunct cancer therapeutic agent excluding proteasome inhibitors, 6) a CK-1 inhibitor and proteasome inhibitor, and optionally, an adjunct cancer therapeutic agent excluding proteasome inhibitors and 7) a CK-1 inhibitor alone, or optionally in combination with adjunct cancer therapeutic agent or PI3K-AKt-mTOR signaling pathway inhibitor, both. In more specific embodiments for options 4, 5, or 7 above, the dual PI3K/CK-1 inhibitor, combination of PI3K-AKT-mTOR signaling pathway inhibitor and CK-1 inhibitor, or CK-1 inhibitor, respectively, may be provided as a lead-in, c-Myc-silencing treatment in a manner to reduce or initiate reduction of c-Myc prior to administration of the adjunct cancer therapeutic agent.

One of the effectors of PI3K is mTOR. mTOR inhibitors are currently approved for the prevention and treatment of organ rejection in transplant recipients, and are also commonly used for the treatment of graft versus host disease (GVHD) in patients undergoing solid organ and bone marrow transplant. A combination of a PI3K-AKT-mTOR signaling pathway inhibitor with a proteasome inhibitor, or a PI3K-AKT-mTOR signaling pathway inhibitor with a CK1 inhibitor is therefore effective in transplant associated complications including organ rejection and GVHD, while at the same time reducing the toxicities associated with current mTOR inhibitors. In a similar manner, such combination strategy is useful for the treatment of other autoimmune disorders.

One of the main pathological factors in rheumatoid arthritis and other autoimmune diseases is activated nuclear factor kappa B (NF-kB) in immune cells. Constitutively activated NF-kB in immune cells is suppressed by proteasome inhibitors, especially immune-proteasome specific inhibitors such as carfilzomib. Therefore, according to certain embodiments, co-administration of a combination of a proteasome inhibitor (e.g. carfilzomib) with either a dual PI3K/CK1 inhibitor, or a CK1 inhibitor, or a PI3K inhibitor will be more effective and safer in the treatment of the following autoimmune diseases, including rheumatoid arthritis, psoriasis, eczema, asthma, multiple sclerosis, inflammatory bowel disease, Crohn's disease, colitis (e.g., ulcerative colitis), systemic lupus erythematosus, myasthenia gravis, Sjögren's syndrome and sclerodema, autoimmune hemolytic anemia, cold agglutinin disease, and IgA nephropathy.

### Proteasome Inhibitors

Examples of proteasome inhibitors useful in accord with the teachings herein include, but are not limited to, the following:

boronic ester or acid such as bortezomib (originally coded PS-341, and marketed as Velcade by Millennium Pharmaceuticals) is the approved name of the chemical entity [(1R)-3-methyl-1-({(2S)-3-phenyl-2-[(pyrazin-2-ylcarbonyl)amino]propanoyl}amino)butyl]boronic acid; or Ixazomib (MLN 2238); (R)-1-(2-(2,5-dichlorobenzamido)acetamido)-3-methylbutylboronic acid;

disulfiram [Disulfanediylbis(carbonothioylnitrilo)]tetraethane];

epigallocatechin-3-gallate (EGCG);

Salinosporamide A: 4R,5S)-4-(2-chloroethyl)-1-((1S)-cyclohex-2-enyl(hydroxy)methyl)-5-methyl-6-oxa-2-azabicyclo[3.2.0]heptane-3,7-dione;

Carfilzomib (PR-171); (S)-4-methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide;

Oprozomib; (ONX-0912); O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide;

CEP-18770: [(1R)-1-[[(2S,3R)-3-hydroxy-2-[[(6-phenylpyridin-2-yl)carbonyl]amino]-1-oxobutyl]amino]-3-methylbutyl]boronic acid;

MLN9708: 4-(carboxymethyl)-2-((R)-1-(2-(2,5-dichlorobenzamido)acetamido)-3-methylbutyl)-6-oxo-1,3,2-dioxaborinane-4-carboxylic acid;

YU 101: (αS)-α-(acetylamino)benzenebutanoyl-L-leucyl-N-[(1S)-3-methyl-1-[[(2R)-2-methyl-2-oxiranyl]carbonyl]butyl]-L-phenylalaninamide;

Marizomib: (NPI-0052); (4R,5S)-4-(2-chloroethyl)-1-((1S)-cyclohex-2-enyl-(hydroxy)methyl)-5-methyl-6-oxa-2-azabicyclo[3.2.0]heptane-3,7-dione;

Epoxomicin: (2S,3S)—N-((2S,3R)-3-hydroxy-1-(((S)-4-methyl-1-((R)-2-methyloxiran-2-yl)-1-oxopentan-2-yl)amino)-1-oxobutan-2-yl)-3-methyl-2-((2S,3S)-3-methyl-2-(N-methylacetamido)pentanamido)pentanamide;

MG132: N-(benzyloxycarbonyl)leucinylleucinylleucinal Z-Leu-Leu-Leu-al; and

Lactacystin: 2-(acetylamino)-3-[({3-hydroxy-2-[1-hydroxy-2-methylpropyl]-4-methyl-5-oxopyrrolidin-2-yl}carbonyl)sulfanyl]propanoic acid.

See also, Crawford L. J., Walker B., Irvine A. E. Proteasome inhibitors in cancer therapy. Journal of Cell Communication and Signaling. 2011; 5(2):101-110. doi:10.1007/s12079-011-0121-7.

### PI3K Inhibitors

Examples of PI3K inhibitors useful for administration for cancer or autoimmune therapies as taught herein are set forth in Tables 1. In a specific embodiment, the PI3K inhibitor used as the therapeutic agent is TGR1202. TGR-1202 (previously known as RP5264) is a highly selective inhibitor for the δ isoform of phosphatidylinositol 3-kinase (PI3K), referred to as PI3Kδ. It is well-tolerated and has greatly reduced hepatoxicity compared to other, less selective PI3K inhibitors and has nanomolar potency.

A phosphatidylinositol 3-kinase (PI3K) inhibitor is a class of drug that inhibits one or more of the four isoforms (α, β, γ, or δ) of the phosphoinositide 3-kinase enzymes. These enzymes are a part of the PI3K-AKT-mTOR signaling pathway, which regulates the cell cycle and is important to the survival of cancer cells. PI3K is constitutively active in some hematologic cancers such as chronic lymphocytic leukemia (CLL). This constitutive activity allows the cells to evade apoptosis. The δ isoform, PI3Kδ, is predominantly expressed in cells of hematologic origin and is largely confined to lymphocytes.

TGR-1202 acts by interfering with the PI3K-AKT-mTOR pathway (inhibiting AKT phosphorylation) to enable cancer cells to undergo apoptosis. TGR-1202 targets PI3Kδ. It has been shown effective in vitro against CLL and is being tested in studies for other hematologic cancers, for example, B cell lymphomas.

The chemical structure of TGR-1202 is given below.

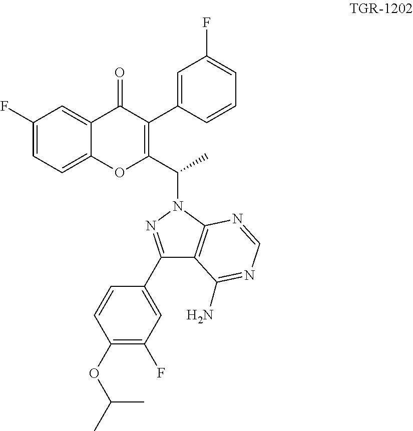 [SMILES: CC(C)Oc1ccc(-c2nn([C@@H](C)c3oc4ccc(F)cc4c(=O)c3-c3cccc(F)c3)c3ncnc(N)c23)cc1F]

Compounds according to the generic structure of Formula I below are also PI3Kδ inhibitors that may be used in accord with the embodiments herein:

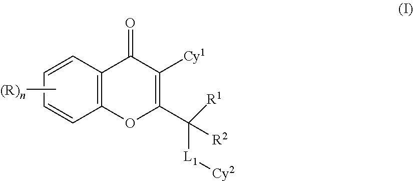

or a tautomer thereof, N-oxide thereof, pharmaceutically acceptable ester thereof, prodrug thereof, or pharmaceutically acceptable salt thereof, wherein each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;

R¹ and R² may be the same or different and are independently selected from hydrogen, halogen, and substituted or unsubstituted C₁₋₆ alkyl, or both R¹ and R² directly bound to a common atom, may be joined to form an oxo group (=0) or a substituted or unsubstituted saturated or unsaturated 3-10 member ring (including the carbon atom to which R¹ and R² are bound), which may optionally include one or more heteroatoms which may be the same or different and are selected from O, NR^{a} and S;

Cy¹ is a monocyclic group selected from substituted or unsubstituted cycloalkyl, substituted or unsubstituted heterocyclic group, substituted or unsubstituted aryl and substituted or unsubstituted heteroaryl;

Cy² is selected from a substituted or unsubstituted heterocyclic group, substituted or unsubstituted aryl and substituted or unsubstituted heteroaryl;

L₁ is absent or selected from —(CR^{a}R^{b})_{q}—, -0-, —S(=0)_{q}-, —NR^{a}— or —C(═Y)—. each occurrence of R^{a} and R^{b} may be the same or different and are independently selected from hydrogen, halogen, hydroxy, cyano, substituted or unsubstituted (C₁₋₆)alkyl, —NR^{c}R^{d} (wherein R^{e} and R^{d} are independently hydrogen, halogen, hydroxy, cyano, substituted or unsubstituted (C₁₋₆)alkyl, and (C₁₋₆)alkoxy) and —OR^{c} (wherein R^{c} is substituted or unsubstituted (C₁₋₆)alkyl) or when R^{a} and R^{b} are directly bound to a common atom, they may be joined to form an oxo group (=0) or form a substituted or unsubstituted saturated or unsaturated 3-10 member ring (including the common atom to which R^{a} and R^{b} are directly bound), which may optionally include one or more heteroatoms which may be the same or different and are selected from O, NR^{d} (wherein R^{d} is hydrogen or substituted or unsubstituted (C₁₋₆)alkyl) or S;

Y is selected from O, S, and NR^{a}; n is an integer from 1 to 4; and q is 0, 1 or 2; are expected to have the same activity and are contemplated as part of the invention.

Certain preferred compounds are those according to Formula II:

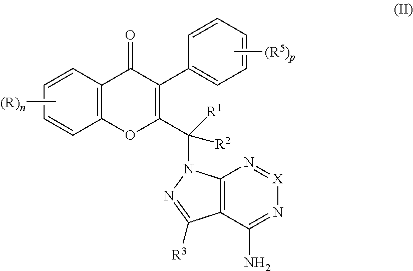

or a pharmaceutically acceptable salt thereof, wherein each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;

R¹ and R² may be the same or different and are independently selected from hydrogen, halogen, and substituted or unsubstituted C₁₋₆ alkyl, or both R¹ and R² directly bound to a common atom, may be joined to form an oxo group (=0) or a substituted or unsubstituted saturated or unsaturated 3-10 member ring (including the carbon atom to which R¹ and R² are bound), which may optionally include one or more heteroatoms which may be the same or different and are selected from O, NR^{a} and S; each occurrence of X is independently selected from CR³ or N; and each occurrence of R³ is independently selected from hydrogen, hydroxy, halogen, carboxyl, cyano, nitro, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenylalkyl substituted or unsubstituted cycloalkenyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(0)R^{x}, —C(S)R^{X}, —C(0)NR^{x}R^{y}, —C(0)ONR^{x}R^{y}, —NR^{y}R^{z}, —NR^{x}CONR^{y}R^{z}, —N(R^{X})SOR^{y}, —N(R^{x})S0₂R^{y}, —(═N—N(R^{X})R^{y}), —NR^{X}C(0)OR^{y}, —NR^{x}R^{y}, —NR^{x}C(0)R^{y}—, —NR^{X}C(S)R^{y}—NR^{x}C(S)NR^{y}R^{z}, —SONR^{x}R^{y}—, —S0₂NR^{x}R^{y}—, —OR^{x}, —OR^{x}C(0)NR^{y}R^{z}, —OR^{x}C(0)OR^{y}—, —OC(0)R^{x}, —OC(0)NR^{x}R^{y}, —R^{x}NR^{y}C(0)R^{z}, —R^{x}OR^{y}, —R^{x}C(0)OR^{y}, —R^{X}C(0)NR^{y}R^{Z}, —R^{x}C(0)R^{x}, —R^{x}OC(0)R^{y}, —SR^{X}, —SOR^{x}, —S0₂R^{x}, and

—ON0₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted heteroaryl, substituted or unsubstituted heteroarylalkyl, substituted or unsubstituted heterocyclic ring, substituted or unsubstituted heterocyclylalkyl ring, or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} (e.g., R^{x} can be hydrogen or substituted or unsubstituted alkyl) or S. each occurrence of R⁵ is hydrogen, C₁₋₆ alkyl or halogen; n is 0, 1, 2, 3 or 4; and p is 0, 1, 2, 3, 4 or 5.

In another embodiment, PI3K inhibitors include those according to Formula I or a tautomer thereof, N-oxide thereof, pharmaceutically acceptable ester thereof, prodrug thereof, or pharmaceutically acceptable salt thereof, wherein each occurrence of R is independently selected from hydrogen, halogen, —OR^{f} (wherein R^{f} is substituted or unsubstituted (C₁₋₆)alkyl), CN, substituted or unsubstituted C.₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;

R¹ and R² may be the same or different and are independently selected from hydrogen, halogen, and substituted or unsubstituted C₁₋₆ alkyl, or both R¹ and R² directly bound to a common atom, may be joined to form a substituted or unsubstituted saturated or unsaturated 3-10 member ring (including the carbon atom to which R¹ and R² are bound), which may optionally include one or more heteroatoms which may be the same or different and are selected from O, NR^{a} and S;

Cy¹ is a monocyclic group selected from substituted or unsubstituted cycloalkyl, substituted or unsubstituted heterocyclic group, substituted or unsubstituted aryl and substituted or unsubstituted heteroaryl;

Cy² is selected from a substituted or unsubstituted heterocyclic group, substituted or unsubstituted aryl and substituted or unsubstituted heteroaryl;

L¹ is selected from —S(═O)_{q}— and —NR^{a}—; each occurrence of R^{a} is selected from hydrogen, halogen, hydroxy, cyano, substituted or unsubstituted (C₁₋₆)alkyl, —NR^{c}R^{d} (wherein R^{c} and R^{d} are independently hydrogen, halogen, hydroxy, cyano, substituted or unsubstituted (C₁₋₆)alkyl, and (C₁₋₆)alkoxy) and —OR^{c} (wherein R^{c} is substituted or unsubstituted (C₁₋₆)alkyl);

n is an integer from 1 to 4; and

q is 0, 1 or 2.

### New Class of CK-1 Inhibitors

Few if any CK-1 inhibitors have been tested in humans. As is described herein, certain compounds currently known as PI3K inhibitors have been discovered to possess CK-1 inhibitory activity as well, i.e., dual PI3K/CK-1 inhibitors. Thus, given the discovery that these PI3K inhibitor compounds possess CK-1 inhibition, they in actuality represent a new class of CK-1 inhibitors that would be suitable for human trials. Based on x-ray crystal structure analysis, TGR-1202 is structurally related to the known CK-1ε inhibitor PF4800567. In silico docking studies targeting the ATP binding pocket of CK1ε showed that TGR-1202 possessed high docking scores in binding modes highly consistent with PF4800567. Equipped with this information, compounds having structural similarity to certain portions of the TG1-1202 compound allow for the identification of CK-1 inhibitors from known compounds, or the design of new compounds having CK-1 activity.

The compounds of Formulas I and II above represent examples of this new class of CK-1 inhibitors. Also, compounds described in WO2015/001491, incorporated by reference, are compounds belonging to this new class of CK-1 inhibitors. In addition, compounds according to Formulas III or Formula IV below represent embodiments of this new class of CK-1 inhibitors:

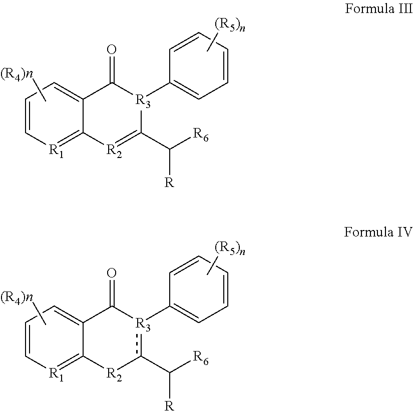

wherein
R is H or any one of groups A-G:

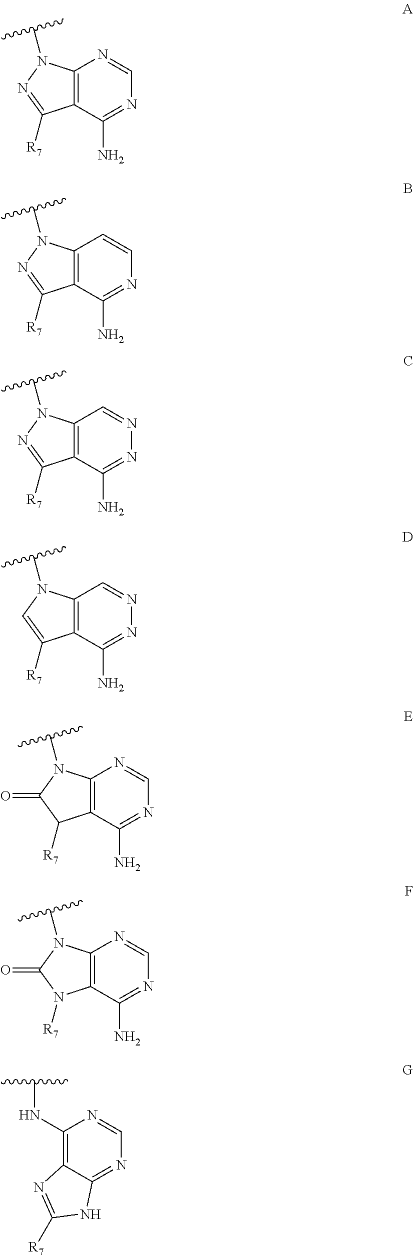

and wherein [symbol]
represents a single or double bond;
R₁ is CH, substituted C or N;

### R₂

in the compound of Formula III is CH, substituted C or N;

in the compound of Formula IV is O, CH₂, substituted C, NH or substituted N;

### R₃

in the compound of Formula III is CH, substituted C or N;

in the compound of Formula IV is
CH, substituted C or N when [symbol] represents a single bond; or
C when [symbol] represents a double bond;
   each R₄ is independently substituted alkyl, unsubstituted alkyl, substituted alkenyl, unsubstituted alkenyl, substituted alkynyl, unsubstituted alkynyl, or halogen; each R₅ is independently substituted alkyl, unsubstituted alkyl, substituted alkenyl, unsubstituted alkenyl, substituted alkynyl, unsubstituted alkynyl, or halogen;
   R₆ is H, Me or Me substituted with halogen;
   R₇ is H or a group selected from any one of groups J, K and H

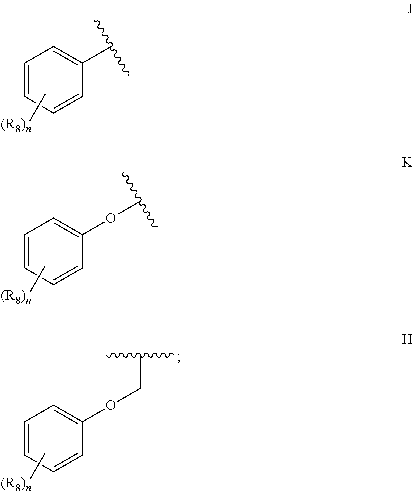

and
each R₈ is independently substituted alkyl, unsubstituted alkyl, substituted O-alkyl,
unsubstituted O-alkyl or halogen;
n,

for R₄ and when R₁ is not N, is 0, 1, 2, 3 or 4;

for R₄ and when R₁ is N, is 0, 1, 2 or 3;

for R₅ is 0, 1, 2, 3, 4 or 5;

for R₈ is 0, 1, 2, 3, 4 or 5;

In certain embodiments, compounds of formula III exclude those wherein at the same time R is group A, R₁ is CH, R₃ is N and R₇ is J.

In other embodiments, compounds of formula IV exclude those wherein at the same time R is group A, R₁ is CH, R₂ is O, R₃ is C, [symbol] represents a double bond, and R₇ is J.

In other certain embodiments, R₇ is not H when R is group G.

In other embodiments, compounds include those of Formulas III and IV with the provisos that
compounds of formula III wherein at the same time R is group A, R₁ is CH, R₃ is N and R₇ is J, are excluded;
compounds of formula IV wherein at the same time R is group A, R₁ is CH, R₂ is O, R₃ is C, [symbol] represents a double bond, and R₇ is J, are excluded;
R₇ is not H when R is group G.

In specific embodiments, one or more of the following apply to Formulas III and IV:

R₁ is N;

R₂ is not O;

R₃ is not N;

R₄ is halogen and n for R₄ is 1 or 2;

R₄ is F and n for R₄ is 1 or 2;

R₄ is F, n for R₄ is 1, and R₄ is located at position 5 of the quinazolin-4-one ring to which it is attached;

n for R₅ is 0;

R₆ is Me; R is not group A;

R is group A;

R₇ is J; R₇ is not J;

n for R₈ is 2, one R₈ is isopropyl or O-isopropyl, and the other R₈ is halogen, preferably F;

R is not group G; and

R₇ is one of the following:

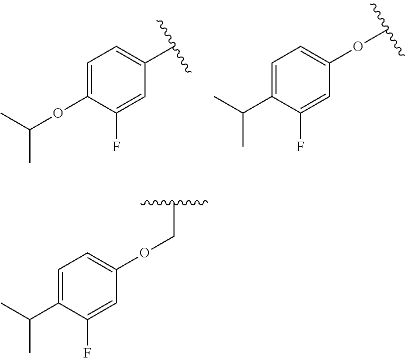 [SMILES: *c1ccc(OC(C)C)c(F)c1 ; *Oc1ccc(C(C)C)c(F)c1 ; *COc1ccc(C(C)C)c(F)c1]

Compounds according to formulas III and IV may be prepared by employing and/or adapting synthetic methodology described in PCT publication Nos. WO2015001491, WO2008/127226, WO2009/088986, WO 2011/055215 and WO 2012/151525, which are incorporated herein by reference. Those skilled in the art would be able to modify the preparation schemes of these references within common general knowledge to produce such compounds.
In a specific embodiment, a new CK-1 inhibitor is CUX-03173 having the following structure:

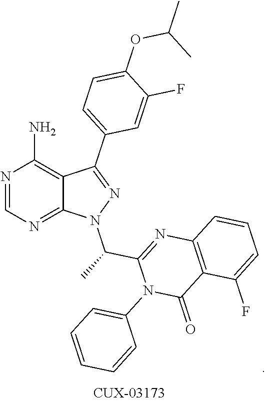 [SMILES: CC(C)Oc1ccc(-c2nn([C@@H](C)c3nc4cccc(F)c4c(=O)n3-c3ccccc3)c3ncnc(N)c23)cc1F ; "."]

It is believed that CUX-03173 is a dual PI3K/CK-1 inhibitor.

### Derivatives

According to certain embodiments, as used herein, derivatives of the specific PI3K inhibitors, proteasome inhibitors, or CK-1 inhibitors as set forth in the tables and discussed herein (example agents) include salts, esters, enol ethers, enol esters, acetals, ketals, orthoesters, hemiacetals, hemiketals, solvates, hydrates, metabolites or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, alk(en)(yn)yl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, alk(en)(yn)yl, aryl, aralkyl, or cycloalkyl. Pharmaceutically acceptable enol esters include, but are not limited to, derivatives of formula C═C(OC(O)R) where R is hydrogen, alkyl, alkenyl, alkynyl, aryl, aralkyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

According to further embodiments, derivatives may include, but are not limited to, specific substitutions of reactive constituents on or emanating from an example agent may include, but are not limited to, one or more of the following: a hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, sulfhydryl, thioalkyl, alkylthio, sulfonyl, C1-C6 straight or branched chain alkyl, C2-C6 straight or branched chain alkenyl or alkynyl, aryl, aralkyl, heteroaryl, carbocycle, or heterocycle group or moiety, or CO2 R7 where R7 is hydrogen or C1-C9 straight or branched chain alkyl or C2-C9 straight or branched chain alkenyl group or moiety.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, alkyl refers to an unbranched or branched hydrocarbon chain. An alkyl group may be unsubstituted or substituted with one or more heteroatoms.

As used herein, alkenyl refers to an unbranched or branched hydrocarbon chain comprising one or more double bonds. The double bond of an alkenyl group may be unconjugated or conjugated to another unsaturated group. An alkenyl group may be unsubstituted or substituted with one or more heteroatoms.

As used herein, alkynyl refers to an unbranched or branched hydrocarbon chain comprising one of more triple bonds therein. The triple bond of an alkynyl group may be unconjugated or conjugated to another unsaturated group. An alkynyl group may be unsubstituted or substituted with one or more heteroatoms.

As used herein, alk(en)(yn)yl refers to an unbranched or branched hydrocarbon group comprising at least one double bond and at least one triple bond. The double bond or triple bond of an alk(en)(yn)yl group may be unconjugated or conjugated to another unsaturated group. An alk(en)(yn)yl group may be unsubstituted or substituted with one or more heteroatoms.

Exemplary alkyl, alkenyl, alkynyl, and alk(en)(yn)yl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl).

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl or isoquinolinyl.

As used herein, “halo,” “halogen,” or “halide” refers to F, Cl, Br or I.

As used herein, base refers to any compound that accepts protons in water or solvent. Thus, exemplary bases include, but are not limited to, alkali metal hydroxides and alkali metal alkoxides (i.e., MOR, wherein M is an alkali metal such as but not limited to potassium, lithium, or sodium and R is hydrogen, alkyl, alkenyl, alkynyl, or alk(en)(yn)(yl) such as but not limited to potassium hydroxide, potassium tert-butoxide, potassium tert-pentoxide, sodium hydroxide, sodium tert-butoxide, lithium hydroxide, etc.); other hydroxides such as but not limited to magnesium hydroxide (Mg(OH)2), calcium hydroxide (Ca(OH)2), or barium hydroxide (Ba(OH)2); alkali metal hydrides (i.e., MH, wherein M is as defined above) such as but not limited to sodium hydride, potassium hydride, or lithium hydride; carbonates such as but not limited to potassium carbonate (K2CO3), sodium carbonate (Na2CO3), potassium bicarbonate (KHCO3), or sodium bicarbonate (NaHCO₃); alkyl ammonium hydroxides, alkenyl ammonium hydroxides, alkynyl ammonium hydroxides, or alk(en)(yn)yl ammonium hydroxides such as but not limited to n-tetrabutyl ammonium hydroxide (TBAH); amines such as ammonia, diethylamine, 2,2,6,6-tetramethyl piperidine (HTMP), tertiary amines (such as but not limited to dimethylethyl amine, diisopropylethylamine, trimethylamine, triethylamine, tributylamine, N-methylmorpholine, N-methylpyrrolidine, 1,8-diazabicyclo[5.4.0]-7-undecene (DBU), 1,5-diazabicyclo[4.3.0]-5-nonene (DBN), or tetramethylenediamine (TMEDA)), aromatic amines (such as but not limited to pyridine, collidine, lutidine, picoline, quinoline, or N,N-dimethylaniline); alkali metal amides such as but not limited to lithium amide, lithium dimethylamide, lithium diisopropylamide (LDA), lithium tetramethylpiperidide (LiTMP), or alkali metal hexamethyldisilazanes (such as but not limited to potassium hexamethyldisilazane, (KHMDS), sodium hexamethyldisilazane (NaHMDS), or lithium hexamethyldisilazane (LiHMDS)); alkyl lithiums, alkenyl lithiums, alkynyl lithiums, or alk(en)(yn)yl lithiums such as but not limited to n-butyl lithium sec-butyllithium, isopropyllithium; alkyl magnesium halides, alkenyl magnesium halides, alkynyl magnesium halides, or alk(en)(yn)yl magnesium halides such as but not limited to methyl magnesium bromide.

As used herein, solvent refers to any liquid that completely or partially dissolves a solid, liquid, or gaseous solute, resulting in a solution such as but not limited to hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone.

As used herein, dehydrating agent refers to any compound that promotes the formation of carboxamides from carboxylic acids, such as but not limited to thionyl chloride, sulfuryl chloride, a carbodiimide, an anhydride or a mixed anhydride, a phenol (such as but not limited to nitrophenol, pentafluorophenol, or phenol), or a compound of Formula (A):

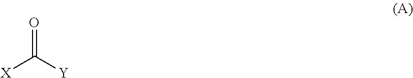

wherein each of X and Y is independently an unsubstituted or substituted heteroaryl group (such as but not limited to imidazolyl, benzimidazolyl, or benzotriazolyl). Examples of dehydrating agents further include, but are not limited to, benzotriazole-1-yl-oxy-tris-(dimethylamino)-phosphonium hexafluorophosphate (BOP), N,N′-carbonyldiimidazole (CDI), 3-(diethoxyphosphoryloxy)-1,2,3-benzotriazin-4(3H)-one (DEPBT), 1-ethyl-3-(3-dimethyllaminopropyl)carbodiimide (EDC), 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 1-hydroxybenzotriazole (HOBt), benzotriazole-1-yl-oxy-tris-pyrrolidino-phosphonium hexafluorophosphate (PyBOP), 2-(1H-benzotriazole-1-yl)-1,1,3,3-tetramethyluronium tetrafluoroborate (TBTU), O-(3,4-dihydro-4-oxo-1,2,3-benzotriazine-3-yl)-N,N,N,N-tetra methyluronium tetrafluoroborate (TDBTU), 3-(diethyloxyphosphoryloxy)-1,2,3-benzotriazin-4(3H)-one (DEPBT), dicyclohexylcarbodiimide (DCC), N,N′-diisopropylcarbodiimide (DIC), or 1-hydroxy-7-azabenzotriazole (HOAt).

As used herein, acid refers to any compound that contains hydrogen and dissociates in water or solvent to produce positive hydrogen ions, as well as Lewis acids, including but not limited to hydrochloric acid, sulfuric acid, phosphoric acid, acetic acid, trihaloacetic acids (such as but not limited to trifluoroacetic acid or trichloroacetic acid), hydrogen bromide, maleic acid, sulfonic acids (such as but not limited to toluenesulfonic acids or camphorsulfonic acids), propionic acids (such as but not limited to (R)-chloropropionic acid), phthalamic acids (such as but not limited to N—[(R)-1-(1-naphthyl)ethyl]phthalamic acid), tartaric acids (such as but not limited to L-tartaric acid or dibenzyl-L-tartaric acid), lactic acids, camphoric acids, aspartic acids, or citronellic acids.

It is to be understood that reactants, compounds, solvents, acids, bases, catalysts, agents, reactive groups, or the like may be added individually, simultaneously, separately, and in any order. Furthermore, it is to be understood that reactants, compounds, acids, bases, catalysts, agents, reactive groups, or the like may be pre-dissolved in solution and added as a solution (including, but not limited to, aqueous solutions). In addition, it is to be understood that reactants, compounds, solvents, acids, bases, catalysts, agents, reactive groups, or the like may be in any molar ratio.

It is to be understood that reactants, compounds, solvents, acids, bases, catalysts, agents, reactive groups, or the like may be formed in situ.

### Enantiomers/Tautomers

Agents also include where appropriate all enantiomers and tautomers of the example agents. The skilled artisan will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Agents may exist as stereoisomers and/or geometric isomers—e.g. they may possess one or more asymmetric and/or geometric centres and so may exist in two or more stereoisomeric and/or geometric forms. Contemplated herein is the use of all the individual stereoisomers and geometric isomers of those inhibitor agents, and mixtures thereof. The terms used in the claims encompass these forms, provided said forms retain the appropriate functional activity (though not necessarily to the same degree).

Agents also include all suitable isotopic variations of the example agent or pharmaceutically acceptable salts thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36Cl, respectively. Certain isotopic variations of the agent and pharmaceutically acceptable salts thereof, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations of the example agents and pharmaceutically acceptable salts thereof of this disclosure can generally be prepared by conventional procedures using appropriate isotopic variations of suitable reagents.

### Solvates

Agents also include solvate forms of the example agents. The terms used in the claims encompass these forms.

### Polymorphs

Agents also include their various crystalline forms, polymorphic forms and (an)hydrous forms. It is well-established within the pharmaceutical industry that chemical compounds may be isolated in any of such forms by slightly varying the method of purification and or isolation form the solvents used in the synthetic preparation of such compounds.

### Prodrugs

Embodiments of the disclosure further include agents in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Metabolites

Also falling within the scope of this invention are the in vivo metabolic products of compounds of example agents. A “metabolite” is a pharmacologically active product produced through metabolism in the body of a specified compound or salt thereof. Such products can result, for example, from the oxidation, reduction, hydrolysis, amidation, deamidation, esterification, deesterification, enzymatic cleavage, and the like, of the administered compound. Accordingly, the invention includes metabolites of example agents, including compounds produced by a process comprising contacting a compound of this invention with a mammal for a period of time sufficient to yield a metabolic product thereof.

Metabolites are identified, for example, by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to a human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolites, so long as they are not otherwise found in vivo, are useful in diagnostic assays for therapeutic dosing of the compounds of the invention.

### Interfering Molecules

Expression of PI3K or CK-1 can be inhibited by a number of means including silencing via antisense, miRNA, shRNA, or siRNA, for example, directed to a portion of the sequence described at the genbank accession numbers provided herein. In one embodiment, an inhibitor of PI3K or CK-1 or proteasomes comprises an interfering molecule, and wherein the interfering molecule comprises a member selected from the group consisting of a phosphothioate morpholino oligomer (PMO), miRNA, siRNA, methylated siRNA, treated siRNAs, shRNA, antisense RNA, a dicer-substrate 27-mer duplex, and combinations thereof.

siRNA molecules can be prepared against a portion of a nucleotide sequence encoding PI3K or CK-1, according to the techniques provided in U.S Patent Publication 20060110440, incorporated by reference herein, and used as therapeutic compounds. shRNA constructs are typically made from one of three possible methods; (i) annealed complementary oligonucleotides, (ii) promoter based PCR or (iii) primer extension. See Design and cloning strategies for constructing shRNA expression vectors, Glen J McIntyre, Gregory C FanningBMC Biotechnology 2006, 6:1 (5 Jan. 2006).

For background information on the preparation of miRNA molecules, see e.g. U.S. patent applications 20110020816, 2007/0099196; 2007/0099193; 2007/0009915; 2006/0130176; 2005/0277139; 2005/0075492; and 2004/0053411, the disclosures of which are hereby incorporated by reference herein. See also U.S. Pat. Nos. 7,056,704 and 7,078,196 (preparation of miRNA molecules). Synthetic miRNAs are described in Vatolin, et al, 2006 J Mol Biol 358, 983-6 and Tsuda, et al 2005 Int J Oncol 27, 1299-306. See also patent document WO2011/127202 for further examples of interfering molecules for targeting CK-1, for example.

### Administration of Enumerated Therapeutic Agents

Certain embodiments involve administering an enumerated agent or combination of enumerated agents to treat cancer, such as c-Myc-overexpressing cancers including hematologic cancers, and more specifically exemplified co-administration of a dual PI3K/CK-1 inhibitor and a proteasome inhibitor, so as to deliver the agent or agents to a subject in need. Other embodiments involve administration of single agents or co-administration two or more agents to treat cancer (e.g. c-Myc-overexpressing cancer) according to the following: 1) a dual PI3K/CK-1 inhibitor and proteasome inhibitor; 2) a PI3K-AKT-mTOR signaling pathway inhibitor inhibitor, CK-1 inhibitor, and proteasome inhibitor; 3) dual PI3K/CK-1 inhibitor, separate CK-1 inhibitor and proteasome inhibitor; 4) a dual PI3K/CK-1 inhibitor and an adjunct cancer therapeutic agent (excluding proteasome inhibitor); 5) a PI3K-AKT-mTOR signaling pathway inhibitor (i.e. PI3K inhibitor, AKT inhibitor, or mTOR inhibitor) and a CK-1 inhibitor and an adjunct cancer therapeutic agent excluding proteasome inhibitors; 6) CK-1 inhibitor alone or in combination with a PI3K-AKT-mTOR signaling pathway inhibitor or proteasome inhibitor, 7) a CK-1 inhibitor in combination with adjunct cancer therapeutic agent. In more specific embodiments for options 4, 5, or 7, the dual PI3K/CK-1 inhibitor, combination of PI3K-AKT-mTOR signaling pathway inhibitor and CK-1 inhibitor, or CK-1 inhibitor, respectively, may be provided as a lead-in, c-Myc-silencing treatment in a manner to reduce or initiate reduction of c-Myc prior to administration of the adjunct cancer therapeutic agent.

Modes of administering include, but are not limited to oral administration, parenteral administration such as intravenous, subcutaneous, intramuscular or intraperitoneal injections, rectal administration by way of suppositories, transdermal administration, intraocular administration or administration by any route or method that delivers a therapeutically effective amount of the drug or composition to the cells or tissue to which it is targeted. Alternatively, routine experimentation will determine other acceptable routes of administration.

One of the effectors of PI3K is mTOR. mTOR inhibitors are currently approved for the prevention and treatment of organ rejection in transplant recipients, and are also commonly used for the treatment of graft versus host disease (GVHD) in patients undergoing solid organ and bone marrow transplant. Accordingly, certain alternative embodiments pertain to (i) administration of a CK-1 inhibitor alone, (ii) co-administration of a combination of one or more approved a PI3K-AKT-mTOR signaling pathway inhibitors with one or more proteasome inhibitors, or (iii) or one or more a PI3K-AKT-mTOR signaling pathway inhibitors with one or more CK1 inhibitors to ameliorate transplant associated complications including organ rejection and GVHD, while at the same time reducing the toxicities associated with current mTOR inhibitors. In a similar manner, such combination strategy is useful for the treatment of other autoimmune disorders.

According to a specific embodiment, provided is a method involving co-administering a therapeutically effective amount of an a PI3K-AKT-mTOR signaling pathway inhibitor and a therapeutically effective amount of a CK-1epsilon inhibitor in a subject who has received an organ transplant (e.g. a bone marrow transplant or stem cell transplant). The subject is one that is typically at risk of GVHD related to the organ transplant or exhibits symptoms of GVHD.

One of the main pathological factors in rheumatoid arthritis and other autoimmune diseases is activated nuclear factor kappa B (NF-kB) in immune cells. Constitutively activated NF-kB in immune cells is suppressed by proteasome inhibitors, especially immune-proteasome specific inhibitors such as carfilzomib. Therefore, the combinations of a proteasome inhibitor (for example, carfilzomib) with either a dual PI3K/CK1 inhibitor, or a CK1 inhibitor, or a PI3K inhibitor is more effective and safer in the treatment of the following autoimmune diseases, including rheumatoid arthritis, colitis, systemic lupus erythematosus, Sjögren's syndrome and sclerodema, autoimmune hemolytic anemia, cold agglutinin disease, and IgA nephropathy.

Another embodiment provided herein is directed to a method that involves administering or co-administering therapeutically effective amounts of (i) a CK-1 inhibitor alone, (ii) a combination of one or more PI3K-AKT-mTOR signaling pathway inhibitors with one or more proteasome inhibitors, or (iii) or one or more a PI3K-AKT-mTOR signaling pathway inhibitors with one or more CK1 inhibitors in a subject, wherein the subject is diagnosed with or exhibits one or more symptoms of an autoimmune disease. In a more specific embodiment, the autoimmune disease is rheumatoid arthritis, psoriasis, asthma, eczema, inflammatory bowel syndrome, Chrohn's disease, colitis (e.g. ulcerative colitis), systemic lupus erythematosus, myasthenia gravis, multiple sclerosis, Sjögren's syndrome and sclerodema, autoimmune hemolytic anemia, cold agglutinin disease, or IgA nephropathy. Not to be bound by theory, it is known that over expression of CK-1ε mimics WNT-signaling, and increased WNT signaling induces release of interleukin 12. Interleukin 12 is known to be a mediating cytokine in autoimmune disorders. Therefore, inhibiting CK-1 likely acts to reduce IL-12 as a mechanism for treating autoimmune disorders.

Typically, agents are administered to a subject in an amount effective to achieve a desired therapeutic effect. The therapeutically effective amount will vary depending on the compound, the disease and its severity and the age, weight, etc., of the subject to be treated. In the context of treating cancer, a therapeutically effective amount refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

In a certain embodiment, a composition of this invention can be administered to a subject who has symptoms of or is diagnosed with a carcinoma. A composition of this invention can be administered prophylactically, i.e., before development of any symptom or manifestation of the disease, disorder or condition. Typically, in this case the subject will be at risk of developing the condition. Treating also may comprise treating a subject exhibiting symptoms of a certain disease, disorder or condition.

Co-administration of a combination of enumerated therapeutic agents, as described herein, may be accomplished by administering a mixed formulation comprising two or more agents (e.g., single composition). Alternatively, the two or more enumerated agents can be administered separately. The co-administration may be conducted by a first step of administering one of the therapeutic agents such as a PI3K Inhibitor, and a second step of administering a second agent such as a proteasome inhibitor, wherein the first and the second administration steps may be conducted simultaneously or sequentially. In case of the sequential administration, the first step and the second step may be performed in any order, and separated by any suitable time interval (e.g., 1-60 seconds, 1-60 minutes, 1-24 hours, or 1-7 days). A first agent, such as a PI3K inhibitor and a second agent, such as a proteasome inhibitor, may be administered in amounts that are therapeutically effective when combined, which amount may be determined by the skilled medical practitioner or medical researcher. Alternatively, as described elsewhere herein, a CK-1 inhibitor can also be co-administered with a PI3K inhibitor or administered in place of a PI3K inhibitor for co-administration with a proteasome inhibitor.

The relevant literature shows that CK1 isoforms can influence the development and progression of tumor cells, although they seem to have different effects depending on the tumor types. Birgit Schittek and Tobias Sinnberg, Molecular Cancer 2014 13:231. The Schittek reference involves a study evaluating survival rates in patients suffering from certain cancer types and whether expression of the alpha, delta or epsilon isoforms of CK-1 is either positively or negatively associated with survival in such patients. The study showed that CK-1 alpha expression had a negative association with survival rates in lung or colon cancers, and liposarcoma. However, CK-1 alpha expression had a positive association with survival rates in breast cancer, B cell lymphoma, lymphocytic leukemia, multiple myeloma. CK-1 delta expression had a negative association with lung cancer and glioblastoma, but had a positive association with survival in breast cancer, astrocytic gliomas, and lymphocytic leukemia. CK-1 epsilon expression had a negative association with survival in B cell lymphoma, lung cancer, and breast cancer but had a positive association with survival rates in gliomas, lung cancer and lymphocytic leukemia. In addition, CK-1 delta was shown to have elevated expression levels in Choriocarcinomas (Stoter M, Bamberger A M, Aslan B, Kurth M, Speidel D, Loning T, et al. Inhibition of casein kinase I delta alters mitotic spindle formation and induces apoptosis in trophoblast cells. Oncogene (2005) 24(54):7964-75); and high grade ductal pancreatic carcinomas (Brockschmidt C, Hirner H, Huber N, Eismann T, Hillenbrand A, Giamas G, et al. Anti-apoptotic and growth-stimulatory functions of CK1 delta and epsilon in ductal adenocarcinoma of the pancreas are inhibited by IC261 in vitro and in vivo. Gut (2008) 57(6):799-806). CK-1episilon was shown to have elevated expression levels in high-grade ductal pancreatic carcinomas (Brockshmidt et al, supra), mammary DCIS (Fuja T J, Lin F, Osann K E, Bryant P J. Somatic mutations and altered expression of the candidate tumor suppressors CSNK1 epsilon, DLG1, and EDD/hHYD in mammary ductal carcinoma. Cancer Res (2004) 64(3):942-51), breast cancer (Shin S, Wolgamott L, Roux P P, Yoon S O. Casein Kinase 1 {varepsilon} Promotes Cell Proliferation by Regulating mRNA Translation. Cancer Res (2014) 74(1):201-11), adenoid cystic carcinoma of the salivary gland (Frierson H F Jr., El-Naggar A K, Welsh J B, Sapinoso L M, Su A I, Cheng J, et al. Large scale molecular analysis identifies genes with altered expression in salivary adenoid cystic carcinoma. Am J Pathol (2002) 161(4):1315-23), epithelial ovarian cancer (Rodriguez N, Yang J, Hasselblatt K, Liu S, Zhou Y, Rauh-Hain J A, et al. Casein kinase I epsilon interacts with mitochondrial proteins for the growth and survival of human ovarian cancer cells. EMBO Mol Med (2012) 4(9):952-63), and tumors of brain, head and neck, bladder, lung, prostate, and salivary gland (Yang W S, Stockwell B R. Inhibition of casein kinase 1-epsilon induces cancer-cell-selective, PERIOD2-dependent growth arrest. Genome Biol (2008) 9(6):R92).

Based on these findings, a combination of an inhibitor for a specific CK-1 isoform can be combined with a PI3K inhibitor and proteasome inhibitor to treat a cancer that is dependent on the complex network of stimulatory signals from the PI3K-AKT-mTOR, CK1, and proteasome pathways to produce overexpression of c-Myc and other pro-survival oncogenes. In a specific embodiment, a PI3K inhibitor, proteasome inhibitor and CK-1 alpha inhibitor are co-administered in therapeutically effective amounts to treat lung cancer, colon cancer or a liposarcoma in a subject in need thereof. In a specific embodiment, the CK-1 alpha inhibitor is lenalidomide.

According to another embodiment, a PI3K inhibitor, a proteasome inhibitor and a CK-1delta inhibitor are co-administered to treat lung cancer or glioblastoma in a subject in need thereof. In a specific embodiment the CK-1 delta inhibitor is PF 670462, TA01, TA02, TAK 715 or LH846, see Table 3.

In a further embodiment, a PI3K inhibitor, a proteasome inhibitor and a CK-1 epsilon inhibitor are co-administered in therapeutically effective amounts to treat lung cancer or breast cancer.

In a further embodiment, a combination of mTOR inhibitors with proteasome inhibitors, or mTOR inhibitors with CK1epsilon inhibitors are co-administered to prevent or treat GVHD, while at the same time reducing the toxicities associated with current mTOR inhibitors. In a similar manner, such combination strategy is useful for the treatment of other autoimmune disorders. Combinations of carfilzomib with either a dual PI3K/CK1 inhibitor, or a CK1 inhibitor, or a PI3K inhibitor are more effective and safer in the treatment of the following autoimmune diseases, including rheumatoid arthritis, systemic lupus erythematosus, Sjögren's syndrome and sclerodema, autoimmune hemolytic anemia, cold agglutinin disease, and IgA nephropathy.

By the co-administration of two or more enumerated agents (e.g. a dual PI3K/CK-1 inhibitor and a proteasome inhibitor), enhanced and synergetic effects can be obtained as compared to the use of either single active ingredient without the other. In some cases, a dose of a first enumerated agent or second enumerated agent typically required to achieve a therapeutic effect can be reduced by at least 5, 10, 20, 30, 40, 50, 60, 70, 80 or even 90 percent to achieve the same effect when the first and second enumerated agents are co-administered.

### Adjunct Cancer Therapy

Disclosed herein is the discovery that inhibition of CK-1epsilon or CK-1 and PI3K can reduce c-Myc in c-Myc-overexpressing cells. The reduction of c-Myc expression makes the c-Myc overexpressing cells more susceptible to other adjunct cancer therapy protocols such as chemotherapy, surgery, radiotherapy, thermotherapy, cancer vaccines, immunotherapy, gene therapy and laser therapy. The data provided herein thoroughly demonstrates that a combination of a dual PI3K/CK-1 inhibitor with a proteasome inhibitor provides a strong cancer killing effect. It is believed that the reduction of the c-Myc expression in the cancer cells makes the cells more susceptible to the cytotoxic effects of the proteasome inhibition. This same effect carries over to other cancer therapeutic agents. Accordingly, certain embodiments pertain to methods that involve administering a CK-1 inhibitor, a dual PI3K/CK-1 inhibitor or a combination of a PI3K-AKT-mTOR pathway inhibitor and CK-1 inhibitor, with an adjunct cancer therapeutic agent to enhance treatment of c-Myc-overexpressing cancer cells.

### Pharmaceutical Formulations

Certain embodiments are directed to pharmaceutical formulations comprising a combination of therapeutically effective amount of an enumerated PI3K-AKT-mTOR signaling pathway inhibitor and a CK-1 inhibitor, and optionally an adjunct cancer therapeutic agent. In select embodiments, formulations are provided that include the following combination of enumerated agents: 1) a dual PI3K/CK-1 inhibitor and proteasome inhibitor; 2) a PI3K-AKT-mTOR signaling pathway inhibitor inhibitor, CK-1 inhibitor, and proteasome inhibitor; 3) dual PI3K/CK-1 inhibitor, separate CK-1 inhibitor and proteasome inhibitor; 4) a dual PI3K/CK-1 inhibitor and an adjunct cancer therapeutic agent (excluding proteasome inhibitor); 5) a PI3K-AKT-mTOR signaling pathway inhibitor (i.e. PI3K inhibitor, AKT inhibitor, or mTOR inhibitor) and a CK-1 inhibitor and an adjunct cancer therapeutic agent excluding proteasome inhibitors; 6) CK-1 inhibitor alone or in combination with a PI3K-AKT-mTOR signaling pathway inhibitor or proteasome inhibitor, 7) a CK-1 inhibitor in combination with adjunct cancer therapeutic agent. In a specific embodiment, the adjunct cancer therapeutic agent either includes or excludes a proteasome inhibitor. Agents useful in therapeutic methods described herein may be provided in a formulation or composition acceptable for administration to a subject. Typically, agent(s) are provided with a pharmaceutically acceptable carrier. “Pharmaceutically acceptable carrier” is intended to include any and all solvents, binders, diluents, disintegrants, lubricants, dispersion media, coatings, antibacterial and antifungal agents, isotonic and absorption delaying agents, and the like, compatible with pharmaceutical administration. The use of such media and agents for pharmaceutically active substances is well known in the art. As long as any conventional media or agent is compatible with the active agent, such media can be used in the compositions of the invention and supplementary active agents or therapeutic agents can also be incorporated into the compositions. A pharmaceutical composition of the invention is formulated to be compatible with its intended route of administration.

Solutions or suspensions can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

As noted above, “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. In the context of treating cancer, a therapeutically effective amount refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

In another embodiment, a therapeutically effective amount refers to that amount of active ingredient which modulates target activity such as PI3K or proteasome activity, or CK-1 activity, compared to that which occurs in the absence of the therapeutically effective dose.

Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents. Clinicians can readily determine the therapeutically effective amount using techniques known in the art.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Preferably, a therapeutic agent reduces expression of a target gene or the activity of a target polypeptide by at least about 10, preferably about 50, more preferably about 75, 90, or 100% relative to the absence of the reagent. The effectiveness of the mechanism chosen to decrease the level of expression of a target gene or the activity of a target polypeptide can be assessed such as by hybridization of nucleotide probes to target-specific mRNA, quantitative RT-PCR, immunologic detection of a target polypeptide, or measurement of target polypeptide activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles.

The combination of therapeutic agents can act synergistically to effect the treatment of cancer. Using this approach, one may be able to achieve therapeutic efficacy with lower dosages of each agent, thus reducing the potential for adverse side effects. Any of the therapeutic methods described above can be applied to any subject in need of such therapy.

### Screening

In accordance with other aspects, there are provided methods for screening PI3K inhibitors to identify selective PI3K inhibitor compounds that will also inhibit the activity of one or more isoforms of the CK-1. Screening methods may involve, cell-free in vitro assays or cell models of cancer (e.g. lymphoma) for example, and determine whether such compounds slow growth of tumor cells or cause them to die. In one specific embodiment, there is provided a method of screening for compounds capable of inhibiting both PI3K and a CK-1 isoform polypeptide. The method comprises determining the activity of a CK-1 isoform with or without contact with a test compound. A test compound that inhibits the activity of the CK-1 isoform and PI3K is identified as a potential dual PI3K/CK-1 inhibitor.

According to another embodiment, disclosed herein are methods for screening of compound libraries to identify selective compounds that modulate activity of PI3K in conjunction with a CK-1 polypeptide. In accordance with another aspect, provided is a method of screening for compounds capable of inhibiting expression of PI3K as well as CK-1.

The compounds tested as dual inhibitors of PI3K and CK-1 can be any small chemical compound, or a biological entity, such as a protein, sugar, nucleic acid or lipid. Typically, test compounds will be small chemical molecules or peptides. Essentially any chemical compound can be used as a potential modulator in the assays of the invention. The compounds can be dissolved in aqueous or organic solutions (e.g., methanol, DMSO, or a mixture of organic solvents). The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

### A. Solid State and Soluble High Throughput Assays

In one embodiment, provided are in vitro soluble assays in a high throughput format. In another embodiment, provided is a soluble or solid phase based in vivo assays in a high throughput format, where the cell or tissue is attached to a solid phase substrate. Optionally, the in vitro assay is a solid phase assay.

In the high throughput assays, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule or cell of interest can be bound to the solid state component, directly or indirectly, via covalent or non-covalent linkage of a tag and or a tag binder. A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders; see SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface, which is reactive with a portion of the tag binder. For example, groups which are suitable for attachment to a longer chain portion would include amines, hydroxyl, thiol, and carboxyl groups Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

### B. Labels and Means of Detection

Detectable labels and moieties can be primary labels (where the label comprises an element which is detected directly or which produces a directly detectable element) or secondary labels (where the detected label binds to a primary label, e.g., as is common in immunological labeling). An introduction to labels, labeling procedures and detection of labels is found in Polak & Van Noorden (1997) Introduction to Immunocytochemistry (2^{nd} ed. 1977) and Handbook of Fluorescent Probes and Research Chemicals, a combined handbook and catalogue Published by Molecular Probes, Inc., Eugene, Oreg. Primary and secondary labels can include undetected elements as well as detected elements.

The particular label or detectable group used in the assay is not a critical aspect of the invention, as long as it does not significantly interfere with the specific binding of an agent used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means.

Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horseradish peroxidase, alkaline phosphatase etc.), spectral colorimetric labels such as colloidal gold or colored glass or plastic (e.g. polystyrene, polypropylene, latex, etc.) beads.

The label may be coupled directly or indirectly to a component of the detection assay according to methods well known in the art. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. As indicated above, a wide variety of labels may be used, with the choice of label depending on sensitivity required, ease of conjugation with the compound, stability requirements, available instrumentation, and disposal provisions.

In general, a detector that monitors a particular probe or probe combination is used to detect the recognition reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling nucleic acids is digitized for subsequent computer analysis.

Preferred labels include those which utilize enzymes such as hydrolases, particularly phosphatases, kinases, esterases and glycosidases, or oxidotases, particularly peroxidases; chemiluminescence (e.g., enzymes such as horseradish peroxidase or alkaline phosphatase with substrates that produce photons as breakdown products; kits available, e.g., from Molecular Probes, Amersham, Boehringer-Mannheim, and Life Technologies/Gibco BRL); color production (using, e.g., horseradish peroxidase, β-galactosidase, or alkaline phosphatase with substrates that produce a colored precipitate; kits available from Life Technologies/Gibco BRL, and Boehringer-Mannheim); hemifluorescence (using, e.g., alkaline phosphatase and the substrate AttoPhos (Amersham) or other substrates that produce fluorescent products); fluorescence (e.g., using Cy-5 (Amersham), fluorescein, and other fluorescent tags, and fluorescent proteins such as Green and Red Fluorescent Protein); antibodies bound to a detectable moiety, and radioactivity. Other methods for labeling and detection will be readily apparent to one skilled in the art. For example, phenotypic changes such as drug resistance can be used as a “label” in the present invention.

Typical enzymes that can be used as reporters or detectable moieties include, e.g., β-galactosidase, luciferase, green or red fluorescent protein, kinase, peroxidase, e.g., horse radish peroxidase, phosphatase, e.g., alkaline phosphatase, and chloramphenicol transferase. The chemiluminescent substrate for luciferase is luciferin. One embodiment of a chemiluminescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures known to those skilled in the art and kits for performing enzyme immunoassays are available as described above.

RNA expression can also analyzed by techniques known in the art, e.g., reverse transcription and amplification of mRNA, e.g., RTQ-PCR, isolation of total RNA or poly A⁺ RNA, northern blotting, dot blotting, in situ hybridization, RNase protection, probing DNA microchip arrays, and the like. In one embodiment, high density oligonucleotide analysis technology (e.g., GeneChip™) is used to identify reporter RNA molecules of the invention, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

In accordance with yet another aspect, there is provided a method for the preparation of a pharmaceutical composition useful for the prevention and/or treatment of a c-Myc-overexpressing cancer, or a symptom thereof. The method comprises identifying a dual PI3K/CK-1 inhibitor in accordance with any method described herein. The method further includes combining of the dual PI3K/CK-1 inhibitor with an acceptable pharmaceutical carrier.

### Detection of CK-1 Expression or Activity and Diagnosis

In certain embodiments, diagnosing, prognosing, or determining progression of cancer involves determining levels CK-1 expression and/or activity in a sample. Typically, the sample involves blood, tissue or cells isolated thereof, or homogenates thereof.

In certain embodiments, disclosed is a method of analyzing a cancer in a subject that involves obtaining a CK-1 expression level from a cancer cell sample obtained from the subject; and comparing the expression level from the cancer cell sample to an expression level of a control. A control in this context may include an CK-1 expression level from a cell sample obtained from or representative of tumor samples from patients showing no evidence of disease, from patients that develop systemic cancer or from healthy individuals without cancer. Observing an elevated CK-1 expression level in the cancer cell sample relative to the control indicates the cancer is susceptible to CK-1 inhibition, such as by CK-1 inhibitor or dual PI3K/CK-1 inhibitor administration, or PI3K inhibitor and CK-1 inhibitor co-administration therapy. Upon determining that the cancer is susceptible to CK-1 inhibition, the method may further involve administering a therapeutically effective amount of CK-1 inhibitor alone, or co-administering a therapeutically effective amount of a dual PI3K/CK-1 inhibitor or CK-1 inhibitor with a therapeutically effective amount of a proteasome inhibitor, or optionally, co-administering a therapeutically effective amount of a PI3K inhibitor, CK-1 inhibitor and proteasome inhibitor.

In another embodiment, provided is a method of monitoring effectiveness of a co-administration chemotherapy in a subject who has cancer that involves determining a pre-treatment CK-1 expression level in a first cancer cell sample from the subject; co-administering a therapeutically effective amount of a dual PI3K/CK-1 inhibitor with a therapeutically effective amount of a proteasome inhibitor, or optionally, co-administering a therapeutically effective amount of a PI3K inhibitor, CK-1 inhibitor and proteasome inhibitor; and then determining a post-treatment CK-1 expression level in a second cancer cell sample from the subject. A reduction in the post-treatment CK-1 expression level relative to the pre-treatment level indicates that the co-administration chemotherapy is effective to treat the cancer.

CK-1 activity or expression can be ascertained at the DNA, mRNA, and protein levels. For example, a reduction in CK-1 expression can be determined based on monitoring the presence of mRNA transcript encoded by the CK-1 gene. Methods known in the art can be used to measure abundance of mRNA transcript, such as PCR, quantitative RT PCR. Another method is a nuclease protection assay, wherein a labeled antisense probe hybridizes in solution to an RNA sample. Following hybridization, single-stranded, unhybridized probe and RNA are degraded by nucleases and intensity of antisense probe is determined for double stranded molecules. In addition, Northern blot assays may be used to detect and ascertain the relative amounts of mRNA transcript in a sample according to conventional Northern blot assay techniques known in the art.

According to other embodiments, RNA can be detected in the cell, in situ. For example, fluorescent in situ hybridization can be used to determine the presence, relative quantity, and spatial distribution of target mRNA in a cell. For example, Single Molecule RNA FISH (Biosearch Technologies, Novato, Calif.) uses multiple short singly labeled oligonucleotide probes complementary to distinct portions of the target sequence. When each probe binds to the single stranded mRNA template, it causes cooperative unwinding of the mRNA, promoting the binding of the additional probes. The net result is the binding of a large multitude of fluorescent labels to a single molecule of mRNA template, providing sufficient fluorescence to reliably locate each target mRNA in a wide-field fluorescent microscopy image.

Detectable probes useful for any of the methods described herein may be constructed according to well-known techniques based on SEQ ID NO. 2, or sequences having high identity thereto.

Determining a level of CK-1 expression may involve detecting/determining a level of CK-1 protein. For example, immunoassays such as Western blot involve immunoprecipitation of protein from a sample according to methods well-known in the art. This is typically followed gel electrophoresis (e.g., SDS-PAGE) of the protein sample. After separation of the proteins, immunocytochemistry and the like can be used to determine the amount of the CK-1 present in the sample. Antibodies, or fragments thereof, that target CK-1 may be used for detection of CK-1.

In another embodiment, CK-1 activity can be determined in a sample based on evaluating the activity levels of CK-1 via a standard enzymatic assay. In one example, a CK-1 activity is conducted at 37° C. in a biological sample (e.g. a cell homogenate) mixed with a reagent mixture containing 25 mM2-(N-morpholino)ethanesulfonic acid, pH 6.5, 50 mM NaCl, 15 mM MgĈ, 2 mg/ml casein, 2 mM EGTA, 100 μM[γ-32P]ATP (100-400 cpm/pmol). Kinetic constants and their standard errors are calculated based on isotope phosphorylation of casein. Alternatively, other suitable synthetic or natural substrates of CK-1 may also be utilized in an enzymatic assay.

### 6. Examples

This invention is not limited to the particular processes, compositions, or methodologies described or exemplified, as these may vary. Although any methods and materials similar or equivalent to those described herein can be used in the practice or testing of embodiments of the present invention, the preferred methods, devices, and materials are described. It will, however, be evident that various modifications and changes may be made thereto without departing from the broader spirit and scope of the invention. Examples are provided below to facilitate a more complete understanding of the invention. The following examples illustrate the exemplary modes of making and practicing the invention, however, the scope of the invention is not limited to specific embodiments disclosed in these Examples, which are for purposes of illustration only. Those skilled in the art will recognize, or be able to ascertain, using no more than routine experimentation, numerous equivalents to the specific embodiments described herein. Such equivalents are considered to be within the scope of this invention.

### Example 1. Materials and Methods

### Cell Culture and Reagents

The cell lines were obtained from ATCC and grown in Iscove Modified Dulbecco Medium with 10% FCS. Fresh medium was added every 2 to 3 days, and the cells were kept at a cell concentration of 0.1 to 1×10⁶/mL. For primary cells, the culture medium was RPMI. The reagents were purchased from Selleck, including carfilzomib, bortezomib, idelalisib/Cal-101. TGR-1202 was provided by TG Therapeutics.

### Cell Free PI3K Activity Assay

Enzyme activity was determined using a PI3K HTRF Assay Kit (Millipore, Billerica, Mass.) with modifications. The PI3 Kinase inhibitor assay works on the established principle that PI3 Kinase phosphorylates PIP2 converting it to PIPS. Fluorescence was measured on a Time Resolved Fluorescent Reader (BMG Labtech., Germany) at excitation and emission wavelengths of 340 & 615 nm respectively.

### Cell Based PI3K Activity Assay

Compound specificity towards PI3Kδ was determined in an IgM-induced B cell proliferation assay. B-cells isolated from blood of healthy subjects were seeded in a 96-well tissue culture plate and incubated with predetermined concentrations of compound for 30 min. Cells were stimulated with 5 μg/ml purified goat anti-human IgM. Growth was assessed using the 3-[4,5-dimethylthiazol-2-yl]-2,5-diphenyltetrazolium bromide (MTT) dye reduction test. For selectivity against PI3K α, β, or γ isoforms, NIH-3T3 or RAW macrophages were seeded in a 6-well tissue culture plate and incubated overnight. Complete medium was replaced with serum-free media the following day and compound at the desired concentrations was added. After 15 min, 20 ng/ml PDGF, 5 μM LPA, or 50 ng/ml c5a was added and incubated for an additional 10 min Cells were lysed and AKT phosphorylation was determined by Western Blotting. Intensity of the bands was determined using ImageJ 1.42q (NIH, USA) and normalized to Actin (loading control).

### Cytotoxicity Assay

Cytotoxicity was performed on cultured cells using Cell Titer Glo, as previously described [22]. Experiments were carried out in 96-well plates, with each treatment in triplicate. Samples were taken at typically 24, 48, and 72 hours after treatment. Cytotoxicity was expressed by the decreasing percentage of live cells in each treatment relative to the untreated control from the same experiment, as a function of time. IC50 (half the maximal inhibitory concentration) for each cell line was calculated using the CalcuSyn Version 2.0 software (Biosoft).

### Calculation of Drug: Drug Synergy

Two independent and similar methods were used to determine drug:drug synergy. First, the Bliss additivism (Bliss independence) model predicts that if Compound X and Compound Y have an additive rather than synergistic effect, the expected fractional inhibition c of the combination X and Y is defined as: c=1−(1−x)*(1−y)=x+y−x*y. The excess over Bliss (EOB) value is determined by subtracting the expected fractional inhibition expected in the additive case from its experimentally determined value z: Δ=z−c. Compound pairs for which Δ≈0 have an additive behavior, whereas compound pairs with positive (or negative) Δ values have synergistic (or antagonistic) behavior. Alternatively, synergy was calculated using relative risk ration (RRR) as described [22]. RRR values below, equal to, or above 1 represent synergistic, additive, or antagonistic effect of the two drugs, respectively.

### Flow Cytometry Analysis of Apoptosis

To study apoptosis, Yo-Pro-1 and propidium iodide (Vybrant apoptosis assay kit #4; Invitrogen) were used, as previously described [23]. A minimum of 10,000 events were acquired from each sample. The fluorescence signals acquired by a FACS Calibur System were resolved by detection in the conventional FL1 and FL3 channels. Cells were considered early apoptotic if Yo-Pro-1-positive but PI-negative, late apoptotic if Yo-Pro-1- and PI positive, and necrotic if only PI-positive. Alternatively, dead cells were detected by flow cytometry using the Alexa Fluor 488 annexin V/Dead Cell Apoptosis Kit from Invitrogen.

### Western Blot

Western blot was performed on whole protein extract from cultured cells under specified treatment conditions, most often for 24 hours. Western blotting was performed according to standard protocols, using the chemiluminescence detection system from Thermo Scientific. The primary antibodies were purchased from Cell Signaling Technology unless specified otherwise, and were against these proteins: AKT, phos-AKT (T308), phos-AKT (S473), mTOR, phos-mTOR, Raptor, STAT3, phos-STAT3, 4EBP1, phos-4EBP1, S6K, phos-S6K, eIF4E, eIF4A, eIF4G, c-Myc, Bcl2, Bcl-xL, PARP. Signals of beta-actin and GAPDH were used as loading control. Goat anti-rabbit or anti-mouse secondary antibodies were purchased from Santa Cruz Biotechnology.

Quantitative PCR (qPCR).

Cells were treated and RNA was extracted using the RNeasy mini kit (Qiagen) according to the manufacturer's protocol. RNA quality and quantity was assessed by nanodrop (Nano Drop 2000c, Thermo Fisher) and normalized RNA quantities were converted to cDNA using the Omniscript Reverse Transcription System according to the manufacturer's protocol (Qiagen). q-PCR's were performed with TaqMan® primers (Applied Biosystems, Thermo Fisher) on a StepOnePlus™ Real-Time PCR System (Applied Biosystems). Relative quantitation of gene expression of target to control genes was determined by the Livak method.

### Plasmids

The pcDNA3 RLUC POLIRES FLUC was a gift from Nahum Sonenberg (Addgene plasmid #45642). The pcDNA3 5′UTRMYC RLUC POLIRES FLUC was constructed by insertion of a PCR-amplified genomic region of the human MYC gene (corresponding to nucleotide+1 to +526 of the 5′ untranslated region) in the Nhe I restriction site upstream of the Renilla Luciferase gene in the pcDNA3 RLUC POLIRES FLUC vector. Translation of the RLUC cistron is cap-dependent, whereas that of the FLUC cistron is directed by the poliovirus IRES and is therefore cap-independent.

Transduction of pCDH-GFP and pCDH-GFP-eIF4E.

Expression vectors for the full-length wild-type eIF4E were generated by inserting the respective coding regions into pCDH-GFP vector. In brief, full-length human eIF4E cDNA was cut out from pHA-eIF4E (Addgene, Cambridge, Mass.) by HindIII/XhoI double digestion followed by filling-in of 5′ overhangs by DNA polymerase I large (Klenow) fragment to form blunt ends. Similarly, pCDH-CMV-MCS-EF1-COPGFP (System Biosciences, Mountain View, Calif.) was digested by EcoRI followed by filling-in of 5′ overhangs then ligated with eIF4E fragment by T4 ligase (NEB, Ipswich, Mass.). Lentivirus were packaged and concentrated by PEG-it (System Biosciences, Mountain View, Calif.) from 293TN cells supernatant after co-transfection of pCDH-CMV-eIF4E-EF1-COPGFP with pPACKH1 packaging plasmids (System Biosciences, Mountain View, Calif.). Myeloma ells (2×10⁶) were transduced with an empty vector lentiviral control pCDH-GFP(EV) or pCDH-GFP-eIF4E construct. Transduced cells were selected by Influx cell sorter (BD Bioscience, San Jose, Calif.) and analyzed by western blotting or cell proliferation assay.

Luciferase Reporter Assay for c-Myc UTR Driven Translation:

Luminescence was measured using the Dual-Luciferase® Reporter Assay System (Promega) on a dual injection Luminometer (Glomax Discover, Promega) according to the manufacturer's protocol. Cap-Dependent Translation rates were determined by the ratio of Renilla to Firefly Luciferase, and RL/FL ratios were compared from control to treated samples.

### Transduction of Plasmids:

Transient transfections of OCI-LY-7 cells were performed by electroporation using the Neon® Transfection System (Invitrogen). Electroporation settings were selected after 24-well optimization was performed according to the manufacturer's protocol. 5×10̂6 Cells and 2 ug of pC-5′-UTRmycRL-IRES-FL were electroporated per 100 ul reaction and incubated for 48 hrs at 37° C./5% CO2. Cells were pooled, counted, and treated with DMSO, combinations of idelalisib and Bortezomib, or combinations of TGR-1202 and Carfilzomib.

### Purification of Primary Lymphoma Cells and Normal Lymphocytes.

Blood from consented patients was drawn into 10 ml EDTA-vacutainers. Blood was mixed with PBS containing 2 mM EDTA and 0.5% BSA in a 1:2 Ratio Blood to PBS. Next fifteen milliliters of Ficoll-Paque Plus (GE Healthcare) was pipetted into 50 ml centrifuge tubes, with blood-PBS mixture layered on top. Tubes were centrifuged at 400×g for 40 min at room temperature with no brake. PBMC's in the buffy coat were collected by pipette and washed in EDTA-BSA PBS. Supernatant was removed and 10 ml ACK Lysis Buffer (Thermo Fisher) was used to remove Erythrocytes. Pellets were again washed in EDTA-BSA PBS, resuspended and counted via automated cell counter. Cytotoxicity and Western Blotting assays were performed according to protocol.

### GEP and Informatics

Sequencing analysis was performed on the samples treated with a PI3K inhibitor (PI3Ki), a proteasome inhibitor (PRi), and the combination (combo). Two models were developed to analyze the data. First, DEseq analysis for each gene using the normalized read counts was run, comparing the drug combination versus each of the two drugs separately. Fold change for each gene and the p-value in the combination versus each of the single drug exposures was calculated. By doing this, the effect of single drug exposure on each gene individually was ruled out. To get a single statistic that represents the effect of drug combination, combining 2 p-values deriving from single comparison is needed. To achieve this, we first transformed pvalue to Z-score with “qnorm” function in R. And then the new combined z-score is obtained by Fisher's method:

Z_{new}=(Z₁+Z₂)/\sqrt{2}

Consequently, genes with a combined z-score larger than 0 are up-regulated by the treatment of drug combination and those with z-score smaller than 0 are down-regulated. All the genes from sequencing results were ranked by their combined z-score. This combined z-score allowed us to rank all genes from the most up-regulated to the most down-regulated. Gene set enrichment analysis (GSEA) was used to evaluate the enrichment for specific genes sets. To further isolate the combination effect from those of the single agents, a generalized linear model was built using the normalized read counts. In this model, the normalized read counts were considered as the dependent variable of three independent variables: the effect of a PI3K inhibitor (PI3Ki), the effect of a proteasome inhibitor (PRi) and the combination effect (combo). Thus, the “glm.nb” function in R was used to fit a generalized linear model for each gene by the following formula:

Count=αE_{PI3Ki}+βE_{PRi}+γE_{combo}+residue

αE_{PI3Ki}+βE_{PRi}+γE_{combo} are all binary variables. They would be 1 if the samples were treated with corresponding drugs, otherwise they would be 0. After obtaining the best generalized linear model, the next focus was the coefficient γ and its p-value, which shows the synergistic effect of TC on each gene. Consequently, genes with a coefficient γ greater than 0 are up-regulated by the treatment of drug combination and those with coefficient smaller than 0 are downregulated. All of the genes were ranked by log(p-value)*sign(γ), which derives a list of all the genes from the most up-regulated to the most down-regulated. Gene set enrichment analysis (GSEA) was then used to evaluate the enrichment for specific genes sets. In addition, the same analysis was performed for other drug combinations including TB, IC, and IB.

### Kinome Profiling

3 PI3Kδ inhibitors were compared on a panel of 365 wild-type protein kinases, using the kinome profiling platform from Reaction Biology as described (Anastassiadis et al., 2011). The substrate for CK1ε was the peptide [KRRRAL[pS]VASLPGL] at 20 μM and [γ-33P]-ATP at 10 μM. The PI3Kδ inhibitors TGR-1202, idelalisib, and IPI-145/duvelisib were used at 1 μM. Control Compound, Staurosporine, was tested in 10-dose IC50 mode with 4-fold serial dilution starting at 20 μM or 100 μM. Alternate Control Compounds were tested in 10-dose IC50 mode with 3-fold serial dilution starting at 20 μM or 100 μM. Information on the conditions of the other 364 kinases in the kinome profiling can be found at the following URL: www.reactionbiology.com/webapps/site/KinaseDetail.aspx.

### CK1ε Kinase Activity Assay

CK1ε kinase activity was determined using the CK1ε enzyme system from Promega, according to the manufacturer's instruction. Full-length recombinant human CK1ε (CK1epsilon) was expressed by baculovirus in Sf9 insect cells using an N-terminal GST tag. ADP-Glo™ Kinase Assay is a luminescent kinase assay that measures ADP formed from a kinase reaction; ADP is converted into ATP, which is a substrate in a reaction catalyzed by Ultra-Glo™ Luciferase that produces light. The luminescent signal positively correlates with ADP amount and kinase activity.

### In Silico Docking

The X-ray crystal structure of CK1ε in its conformation in complex with PF4800567 (PDB accession code 4HNI) was used as a target for in silico docking. The pdb structure was prepared using the Protein Preparation Wizard tool (Sastry et al., 2013) in Maestro release 2015-3 (Maestro 10.3, Schrodinger, LLC, New York, 2015). Notably, PF4800567 was removed from the structure, hydrogen atoms were added, hydroxyl and amide groups (in Ser, Thr, Asn, and Gln), and His protonation states were optimized based on their environment. TGR-1202, Idelalisib, CUX-03166 and CUX-03173 structures were prepared using Ligprep (Ligprep, version 3.5, Schrodinger, LLC, New York, N.Y., 2015). TGR-1202, Idelalisib, CUX-03173 and CUX-03166 were flexibly docked into the ATP binding pocket of the CK1 structure using Glide SP (Standard Precision) (Friesner et al., 2004; Repasky et al., 2007), with no constraint. The binding site was defined by the position of PF4800567. For the case of Idelalisib and CUX-03166, in parallel with docking, the molecules were superposed to PF48000567 and CUX-03173 respectively, using the Superposition tool in Maestro.

### CK1ε Autophosphorylation Assay

The assay was modified from the previously described methods (Cegielska et al., 1998; Cheong et al., 2011; Rivers et al., 1998). Briefly LY10 cells were grown at a cell density of 3×105 cells per milliliter and treated with DMSO, 1 μM PF670462, 1 μM PF4800567, 25 μM TGR-1202, 25 μM idelalisib for 1 h before addition of 50 nM of calyculin A (Sigma-Aldrich) to the culture media. Cells were harvested after 15-60 minutes of treatment by calyculin A for Western blot analysis. Phosphorylation of casein kinase CK1ε was measured by using anti-CK1ε. Protein phosphatase 2A-A subunit (PP2A-A) was used as a loading control.

### Example 2. TGR-1202 is a Novel PI3Kδ Inhibitor Whose Activity and Isoform Selectivity are Comparable to Idelalisib

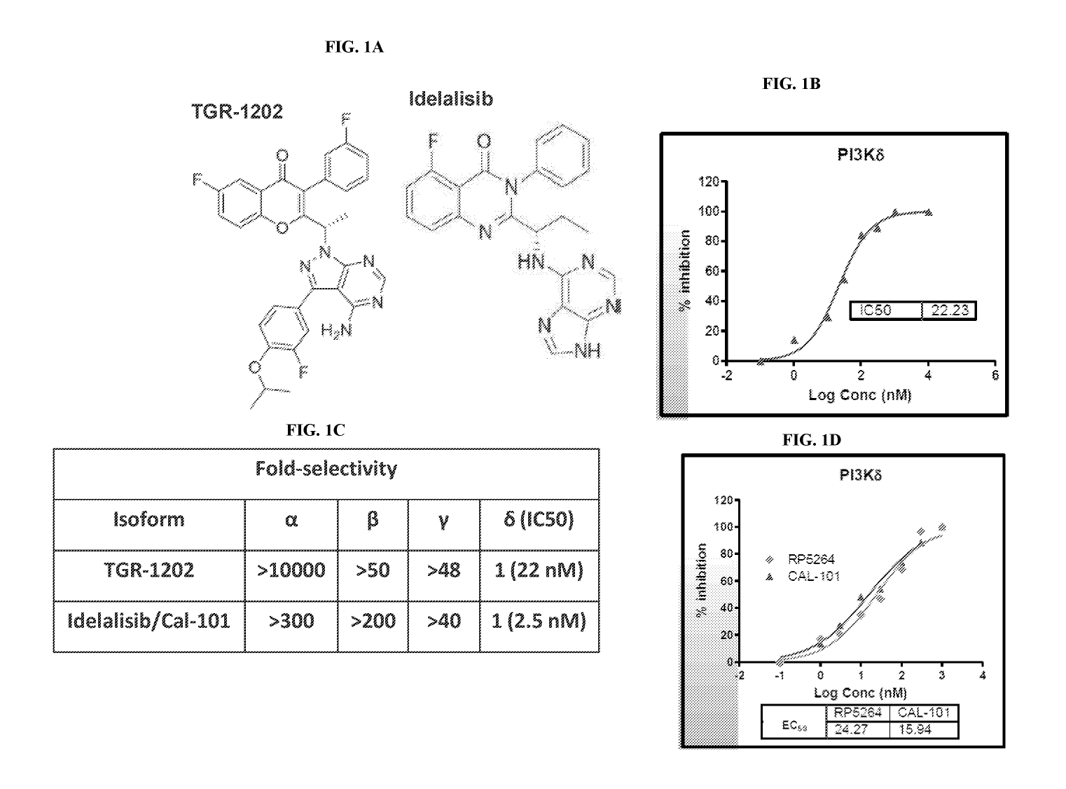

Idelalisib/Cal-101 is a selective PI3Kδ inhibitor with only modest activity in aggressive lymphoma in preclinical studies [23, 24], and is approved for the treatment of indolent B-cell non-Hodgkin lymphoma (iNHL) and chronic lymphocytic leukemia (CLL) [25, 26]. TGR-1202 is a novel PI3Kδ inhibitor with a structure distinct from idelalisib (FIG. 1A). Notably, TGR-1202 does not have the nitrogen heterocyclic ring structure that is present in idelalisib. TGR-1202 is currently in phase I clinical studies and has demonstrated excellent safety and promising clinical activity in iNHL and CLL, and limited activity in aggressive lymphoma [27]. In the cell free system, TGR-1202 potently inhibited recombinant PI3Kδ, with a half maximal inhibitory concentration (IC50) at 22 nanomolar (nM) (FIG. 1B). In contrast, the IC50 values of TGR-1202 for PI3Kα, PI3Kβ, and PI3Kγ were 10000, 50, and 48 times higher, respectively (FIG. 1C). These results establish TGR-1202 as a highly selective PI3Kδ inhibitor. The selectivity of TGR-1202 for PI3Kδ was in the same range as that of idelalisib (FIG. 1C). The IC50 values of idelalisib for PI3Kδ were 2.5 nM, suggesting that idelalisib is significantly more potent than TGR-1202 against PI3Kδ by the cell-free assay using recombinant PI3Kδ. In contrast, in a cell based assay of PI3Kδ [24] the activity of endogenous PI3Kδ signaling was measured by IgM-induced B cell proliferation. The IC50 values of TGR-1202 and idelalisib were 24 nM and 16 nM, respectively (FIG. 1D). These results demonstrated that TGR-1202 is a novel, highly selective PI3Kδ inhibitor, with comparable potency to idelalisib in cell based assays using normal B cells or lymphoma cells.

### Example 3. TGR-1202 and Carfilzomib Demonstrated Superior Activity and Synergy Among Four Combination Pairs of PI3K and Proteasome Inhibitors in DLBCL

The pharmacologic interaction of 2 PI3Kδ inhibitors (TGR-1202 and idelalisib) with 2 FDA approved proteasome inhibitors (carfilzomib and bortezomib) was studied, using a high throughput screening (HTS) platform. Four combination pairs were studied in the DLBLC ABC subtype cell line LY10 (FIG. 2A), including TGR-1202+carfilzomib “T&C” (left upper panel), CAL-101/idelalisib+carfilzomb “C&C” (right upper panel), TGR-1202+bortezomib “T&B” (left lower panel), and CAL-101+bortezomib “C&B” (right lower panel). For every combination pair, each of the two study drugs were given as single agents at 10 concentrations and in combination at 100 conditions resulting from 10×10 pairing of the two drugs. Idelalisib and TGR-1202 were given at the same concentrations ranging from 1 to 15 micromolar (μM), which produced comparable and modest levels of growth inhibition, ranging from 15 to 30%. Carfilzomib was given at concentrations ranging from 1 to 2 nM, which produced up to 35% inhibition. Bortezomib was given at concentrations ranging from 1 to 8 nM: from 1 to 3 nM bortezomib did not produce significant inhibition, and from 4 to 8 nM bortezomib produced 42-90% inhibition.

In the T&C combination (FIG. 2A), there was a marked increase of inhibition in essentially all of the 10×10 combination conditions compared to the single agents at the same concentrations. The increase of growth inhibition by the combination conditions occurred as a function of increasing concentrations of either of the two drugs, namely TGR-1202 and carfilzomib. Similarly but to a less degree, in the C&C combination, the combination conditions produced higher levels of growth inhibition than the single agent CAL-101 or carfilzomib, in a manner proportional to the increasing concentrations of either single agent. In contrast, the T&B combination produced levels of inhibition that were only moderately higher than those achieved by bortezomib as a single agent, and only when bortezomib was given at the higher concentration range, namely 4-8 nM. Finally in the C&B combination, CAL-101 did not add any significant cytotoxicity to bortezomib at any concentration.

The Bliss additivism model was used to calculate the expected inhibition of two drugs that are purely additive [28] (Example 1). Next, the Excess over Bliss (EOB) values were calculated by subtracting the percentage of expected inhibition of an assumed additive combination condition from the observed inhibition. EOB values above, at, and below 0 indicate synergy, additivism, and antagonism, respectively. Higher EOB values above 0 are consistent with higher levels of drug synergy. FIG. 2B demonstrates that the combination pair TGR-1202+carfilzomib (left upper panel, same layout as in FIG. 2A) was highly synergistic in essentially all 10×10 combination conditions. CAL-101 and carfilzomib were synergistic (right upper panel), but to a less degree and only at higher concentrations of CAL-101. In contrast, TGR-1202 and bortezomib were rarely synergistic, while CAL-101 and bortezomib were not synergistic at all according to the Bliss model. These results demonstrate that individual proteasome inhibitors and PI3Kδ inhibitors are not equivalent in their synergistic interactions. In the DLBCL ABC subtype cell line T&C was clearly the most synergistic combination, followed in order by C&C, T&B, and finally C&B, in essence supporting the superior contributions of the TGR-1202 and carfilzomib in these respective combinations.

### Example 4. TGR-1202 and Carfilzomib were Consistently the Most Synergistic Pair Among Four Combinations of PI3K and Proteasome Inhibitors in Aggressive B- and T Cell Lymphomas and Multiple Myeloma

It was investigated whether combining PI3K and proteasome inhibitors would be synergistic in 4 other DLBCL cell lines. The four combination pairs demonstrated distinctly different levels of synergy in the following descending order: T&C>C&C>T&B>C&B, which was consistent across all five DLBCL cell lines, including LY10, SUDHL2, LY1, SUDHL4, and LY7 (FIG. 3). For simplicity, FIG. 3A-3D depicts the striking difference between two combination pairs, TGR-1202+carfilzomib (T&C) versus CAL-101+bortezomib (C&B) in 4 DLBCL cell lines. The midlines of the graphs indicate where observed inhibition was equal to the expected inhibition if the two study drugs were merely additive. The vast majority of the 100 combination conditions of T&C produced observed inhibition that was substantially higher than the expected inhibition. In contrast, the observed inhibition caused by C&B was by and large no more than the expected inhibition in any of the four DLBCL cell lines. These results demonstrated that TGR-1202 and carfilzomib exhibit highly synergistic inhibition of DLBCL cells, regardless of whether they were the ABC or GCB subtype. The other three combination pairs are associated with less frequent and lower levels of synergy in DLBCL.

Interestingly, CAL-101/idelalisib and bortezomib were merely additive in two mantle cell lymphoma (MCL) cell lines, including Jeko-1 and Z138 (FIG. 3E-F), despite their acknowledged activity in the disease. In contrast, TGR-1202 and carfilzomib were highly synergistic at all 100 combination conditions in both MCL cell line models. Extending these observations to T-cell lymphoma (TCL), FIG. 3G-3J demonstrates that TGR-1202 and carfilzomib were potently synergistic in 2 immature T-cell acute lymphoblastic leukemia (T-ALL) cell lines (PF-382 & P12) and 2 mature cutaneous TCL cell lines (HH & H9). In contrast, idelalisib and bortezomib were less synergistic and less effective in all four cell lines representing aggressive TCL. In the MM cell line MM.1S, T&C was also more synergistic than C&B. These data were consistent in that they confirm the unique synergistic interaction of TGR-1202 and carfilzomib in contrast to all other doublets, in a manner that is not due to merely changes in the concentration: effect relationship.

To confirm the synergistic inhibition in the above experiments was associated with killing of the cancer cells, two independent assays were performed, including cleavage of poly (ADP-ribose) polymerase (PARP) [29] and activation of caspase 3/7. FIG. 3L-N demonstrates that the combination pair TGR-1202+carfilzomib was more effective than any single agents or the combination pair Cal-101+bortezomib in inducing PARP cleavage in the DLBCL cell lines LY10 an LY7, and the T-ALL cell line PF382. FIG. 3O demonstrates that TGR-1202 at 1 μM markedly enhanced the ability of carfilzomib to activate caspase 3/7. In contrast, Cal-101 even when used at 5 μM, failed to augment caspase activation caused by bortezomib. Collectively, the above results demonstrated that the PI3Kδ inhibitor TGR-1202 and proteasome inhibitor carfilzomib were highly synergistic in potently inhibiting the growth and survival of cancer cells representing a broad panoply of aggressive B- and T-cell lymphomas as well as multiple myeloma. Other combination pairs of PI3Kδ and proteasome inhibitors proved to be substantially less synergistic in these models.

### Example 5. TGR-1202 and Carfilzomib in Combination Markedly Inhibited Signaling in the mTOR-eIF4F-Myc Axis in Models of B- and T-Cell Lymphoma

To understand the mechanistic basis of the synergy of PI3Kδ and proteasome inhibitors, it was explored how these compounds affected the PI3K-AKT-mTOR pathway in DLBCL. The LY10 cell line was treated with the drugs as single agents and in combination, with the concentration chosen so that the single agents produced a comparable level of inhibition (FIG. 4A). The combination pair TGR-1202+carfilzomib was highly synergistic with a calculated EOB value of 31. The pair Cal-101+bortezomib was not synergistic, with an EOB value of 3 only. At the equimolar concentration, i.e. 3 μM, Cal-101 was more effective than TGR-1202 in inhibiting the phosphorylation of AKT, as well as on reducing the protein level of Raptor, a component of the mTORC1 complex, in the DLBCL cell line LY10 (FIG. 4A, upper panel). Carfilzomib and bortezomib exhibited comparable and modest inhibition of these two signals. Both combinations pairs, namely idelalisib+bortezomib and TGR-1202+carfilzomib, were able to completely suppress AKT phosphorylation, and markedly inhibited the expression of Raptor (FIG. 4A, upper panel). The comparable levels of inhibition on AKT and Raptor by the two combination pairs could not explain why one pair was synergistic, while the other not.

mTOR stimulates the phosphorylation of STAT3, p70S6K, and 4EBP1, which regulate overlapping but discrete downstream pathways. Both TGR-1202 and Cal-101 were able to moderately inhibit the phosphorylation of 4EBP1, and marginally inhibited the phosphorylation of STAT3 and p70S6K, in the LY10 cells (FIG. 4A, middle panel). At the equimolar concentration of 2 nM, bortezomib and carfilzomib caused marginal or no inhibition of these pathways. The combination pair CAL-101+bortezomib did not inhibit the phosphorylation of 4EBP1 at all, mildly inhibited the phosphorylation of STAT3, and markedly inhibited the phosphorylation of p70S6K. In contrast, the combination of TGR-1202+carfilzomib potently inhibited the phosphorylation of 4EBP1, STAT3, and p70S6K in the DLBCL cell line LY10 (FIG. 4A, middle panel). These results suggested that the different levels of synergy in the two combination pairs may be attributed in part to their differential effects on the phosphorylation of 4EBP1. Potent inhibition of 4EBP1 phosphorylation by TGR-1202+carfilzomib could lead to markedly increased sequestration of eIF4E by it cognate inhibitor, the dephosphorylated 4EBP1.

eIF4E as an essential subunit of the eIF4F complex is involved in cap dependent translation of mRNA. Furthermore, eIF4F forms a feed-forward loop with c-Myc by stimulating the translation of c-Myc, and c-Myc in turn activates the transcription of the eIF4F subunits. Therefore, the effects of the PI3K and proteasome inhibitors on the protein levels of eIF4F and some of the cancer related genes known to depend on eIF4F for efficient translation were investigated, including c-Myc and HIF1α. Neither the PI3K inhibitors nor the proteasome inhibitors as single agents had any significant effect on the expression level of eIF4E, eIF4A, or eIF4G1 (FIG. 4A, lower panel). The combination pair CAL-101+bortezomib did not inhibit eIF4E, eIF4A, or eIF4G1. In contrast, TGR-1202 and carfilzomib in combination markedly inhibited the level of eIF4A and eIF4G1. Furthermore, the PI3K inhibitors and proteasome inhibitors as single agents only mildly inhibited the level of c-Myc, and did not inhibit HIF1α.

Similarly, the combination pair CAL-101+bortezomib produced mild inhibition of Myc, and no effect on HIF1α. In contrast, the combination pair TGR-1202+carfilzomib was able to dramatically reduce the protein level of c-Myc and HIF1α(FIG. 4A, lower panel). The above results implicate disruption of the mTOR-eIF4F-Myc axis as a potential mechanism for the marked synergy of TGR-1202 and carfilzomib in the LY10 cell line (ABC subtype). FIG. 4B demonstrated that in the DLBCL cell line LY7 (GCB subtype), the PI3K inhibitors moderately inhibited the phosphorylation of 4EBP1, and the proteasome inhibitors exerted a mild to moderate inhibitory effect on the phosphorylation of 4EBP1. These drugs as single agents had no effect on the protein level of c-Myc. The combination pair CAL-101+bortezomib had essentially the same effect as bortezomib alone on 4EBP1 and Myc. In contrast, the combination pair TGR-1202+carfilzomib markedly inhibited the phosphorylation of 4EBP1 and the expression of c-Myc.

The effects of these drugs in the T-ALL cell line PF382 were examined FIG. 4C demonstrates that at 5 uM, neither TGR-1202 nor idelalisib affected the phosphorylation of AKT, phosphorylation of mTOR, the protein levels of mTOR and Raptor (FIG. 4C, upper panel). At 2.5 nM, the proteasome inhibitors carfilzomib or bortezomib did not significantly change any signals involved in the PI3K-AKT-mTOR cascade. Both combination pairs effectively inhibited the phosphorylation of AKT. Interestingly, only the combination pair TGR-1202 and carfilzomib, but not CAL-101+bortezomib, was observed to cause marked decrease in the phosphorylation of mTOR, and the protein levels of mTOR and Raptor (FIG. 4C, upper panel). Similarly, among the seven treatment conditions, only the combination pair TGR-1202+carfilzomib was able to markedly down-regulate the phosphorylation of STATS, 4EBP1, and S6K (FIG. 4C, middle panel), and substantially inhibited the expression level of c-Myc, HIF1a, and Bcl-xL (FIG. 4C, lower panel). Collectively, these results demonstrated that select combinations of PI3K and proteasome inhibitors, such as TGR-1202+carfilzomib, disrupt mTOR signaling, leading to dephosphorylation of 4EBP1, and potent inhibition of the c-Myc protein level.

### Example 6. TGR-1202 and Carfilzomib in Combination Potently Inhibited the Cap Dependent Translation of c-Myc

Given that c-Myc has a short half-life of about 30 minutes, the impact of the combinations on translation was investigated. C-Myc is regulated at the levels of transcription and translation, and is further subject to phosphorylation and proteasome mediated degradation. FIG. 5A demonstrated that potent reduction of c-Myc protein was associated exclusively with the highly synergistic combination pair TGR-1202+carfilzomib in the DLBCL cell line LY10. The single agents and other combination pairs caused only mild to moderate reduction of the c-Myc protein level. FIG. 5B demonstrated that none of the combination pairs caused any decrease in the mRNA level of c-Myc when compared with the untreated control. In contrast, the mRNA level of a c-Myc target gene, LDH-A, was reduced most effectively by the synergistic combination of TGR-1202+carfilzomib (FIG. 5C). The level of LDH-A mRNA was also significantly reduced by the combination pairs TGR-1202+bortezomib and Cal-101+carfilzomib, but interestingly not by Cal-101+bortezomib. None of the combination pairs significantly reduced the expression of PKM2. These results suggested that the down-regulation of Myc by TGR-1202+carfilzomib occurs at the level of decreased translation rather than transcription, leading to reduced transcription of MYC target genes like LDH-A. To further confirm this finding, a bi-cistronic luciferase reporter was designed as shown in FIG. 5D. Translation of renilla luciferase (LucR) is cap-dependent and requires eIF4F, and is further regulated by the 5′ UTR of C-MYC. In contrast, translation of firefly luciferase (LucF) is not cap dependent as it has the Polio virus internal ribosome entry site (IRES), and is less dependent on the translation initiation factors. This reporter allows us to determine the relative efficiency of cap dependent translation downstream of the 5′ UTR of MYC, using the ratio of renilla luciferase divided by firefly luciferase (R/F Luc). Unfortunately, the lymphoma cell line LY10 was resistant to the transduction of plasmids. Another DLBCL cell line LY7 was used. The synergistic combination pair TGR-1202+carfilzomib markedly decreased the protein, but not the mRNA level of c-Myc in LY7 in the same way as in LY10 (FIGS. 5E & 5F). FIG. 5G demonstrated that combining TGR-1202 at 5 μM and carfilzomib at 5 nM preferentially decreased the R/F Luc ratio when compared to the untreated control (p=0.000013) or the combination of Cal-101 at 5 μM and bortezomib at 5 nM (p=0.0013). TGR-1202 and carfilzomib in combination potently inhibited CAP dependent translation of MYC in a fashion that cannot be capitulated by the C+B combination or single agents, establishing the unique mechanisms of action of this doublet.

### Example 7. TGR-1202 and Carfilzomib in Combination Potently Inhibit the c-Myc Transcription Program

If the potent reduction of the c-Myc protein level by TC is a primary cause instead of a secondary effect of the combination's cytotoxicity, then transcription of c-Myc dependent genes will be specifically inhibited. Gene expression profile (GEP) was performed studies by RNA-seq in the DLBCL LY10 cells treated by the vehicle control, TGR-1202, idelalisib, carfilzomib, bortezomib, and the 4 combinations including TC, TB, IC, and IB for 24 h. DEseq analysis was run for each gene comparing the drug combinations versus each of the two contributing single agents separately. Fold change for each gene and the p-value in the combination versus each of the single drug exposures were calculated. Next, combined were the 2 p-values to calculate a combined Z score, which were used to rank list the genes according to the up- or down-regulation at the transcription level by the combinations.

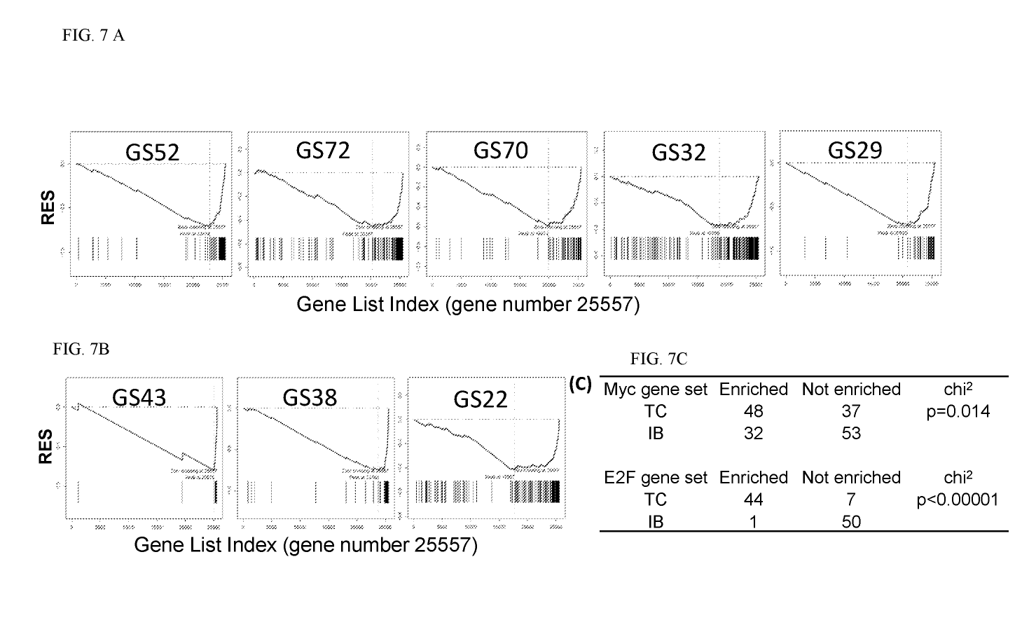

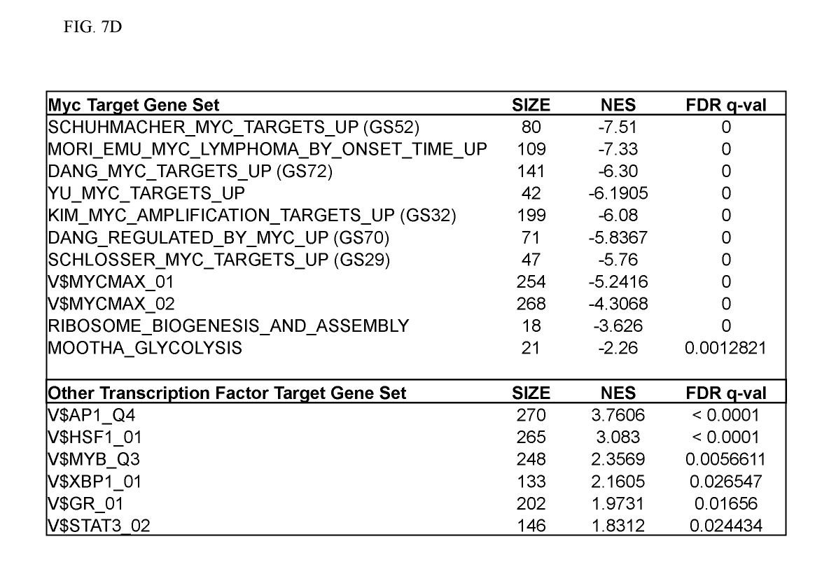

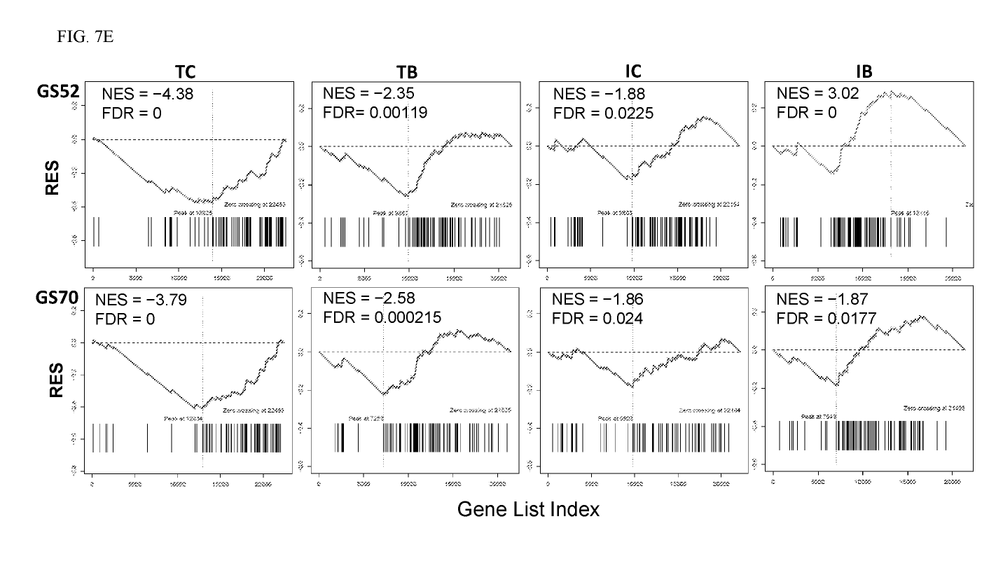

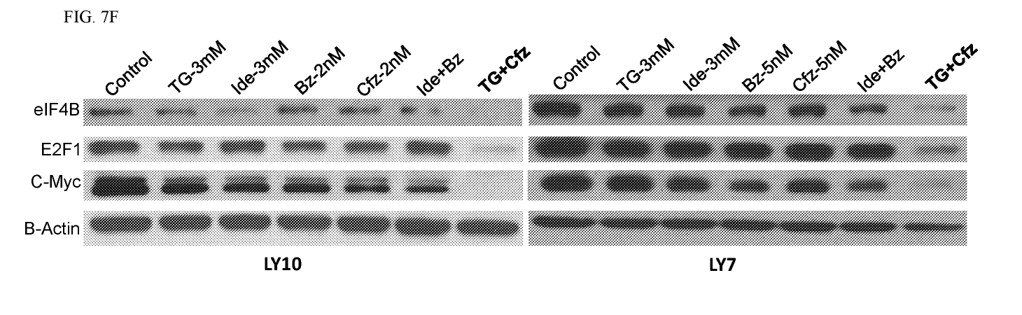

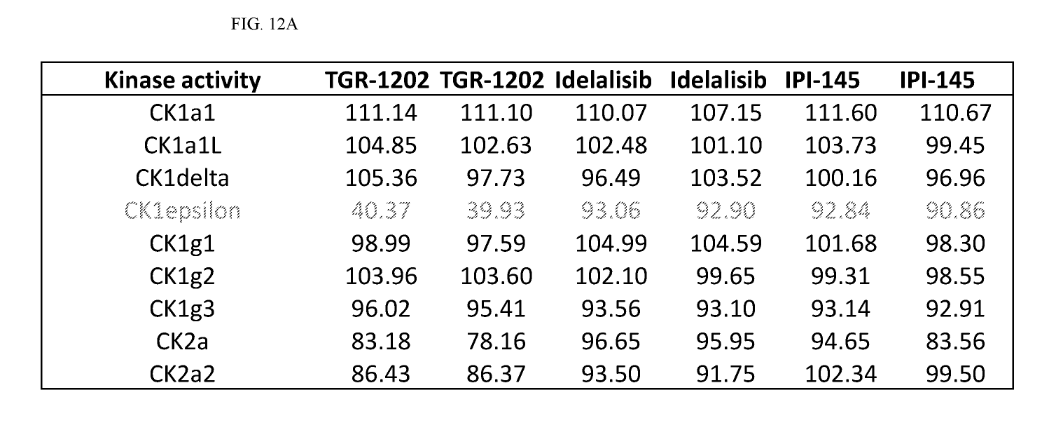

Gene set enrichment analysis (GSEA) was performed to evaluate the enrichment of c-Myc target genes using the annotated gene sets in the Molecular Signatures Database (MSigDB). FIG. 7A demonstrates the Running Enrichment Score (RES) of c-Myc targets, including the 4 “canonical” Myc target gene sets (GS52, GS72, GS32, and GS29) selectively downregulated by the BRD4 inhibitor JQ-1 (Delmore et al., 2011) and an additional gene set GS70. In lymphoma cell treated by the TC combination, the 5 Myc gene sets reached their peak score at the bottom of ranked gene list (normalized enrichment score (NES)<0 and false discovery rate (FDR) q-value=0, FIG. 11A&6D), suggesting that the drug TGR-1202 and carfilzomib in combination exhibits a negative effect on these c-Myc dependent target genes. As JQ-1 has been shown to downregulate E2F target genes, it was investigated whether the effect of the TC combination on the E2F transcription program. FIG. 7 demonstrates that 3 E2F gene sets (GS43, GS38, and GS22) were enriched among the downregulated genes (NES<0 and FDR q-val=0). GSEA was conducted on all the Myc and E2F target gene sets, and found that 48 of the 85 Myc target gene sets and 44 of the 51 E2F target gene sets are significantly down-regulated by the drug combination TC (NES<0 and FDR q-val<0.05, FIG. 7D). In contrast, most unrelated sets have smaller NES and/or larger FDR values. These results indicate that the synergistic combination TC specifically inhibit the c-Myc and E2F transcription programs.

In FIG. 7 it has been shown that the TC combination completely eliminated the protein expression of c-Myc, and the IC, TB, and IB combinations modestly decreased the protein level of c-Myc. Not surprisingly, it was found that the least synergistic IB combination did also downregulate the 5 Myc target gene sets evaluated in FIG. 7A. However, IB downregulated only 32 Myc gene sets and 1 E2F gene sets, compared to 48 Myc gene set and 44 E2F gene sets downregulated by the TC combination. The differential effects by TC and IB on Myc and E2F were statistically significant (p=0.014 for Myc and p<0.00001 for E2F) (FIG. 7C). These results support that silencing of the c-Myc and E2F transcription programs is likely a specific and a primary event for the highly synergistic TC combination but a less specific and likely secondary effect for the non-synergistic IB combination.

To further distinguish the 4 combination pairs in terms of their effects on the c-Myc transcription program, it was attempted to identify and focus on genes that were differentially regulated by the TC, TB, IC, and IB combinations compared to the respective single agents in these combinations. To do this, a generalized linear model was built to calculate a coefficient and p-value which showed the synergistic effect of the drug combinations on each gene. The coefficient p-values were then calculated by multiplying the coefficient and the logarithmic p-values, which were used to rank list the genes according to the up- or down-regulation at the transcription level by the combinations. This analysis generated the differentially expressed genes for the TC, TB, IC, and IB combinations, which were then rank listed and used to run GSEA on two c-Myc gene sets, GS52 and GS70 (FIG. 7E). The most synergistic combination, TC, downregulated both GS52 and GS70 with the lowest NES scores and FDR values among the 4 combinations. There was a consistent trend of decreasing significance of downregulation of these c-Myc gene sets by the TC, TB, IC, and IB combinations, with IB showing the least significant downregulation of GS52 (NES=3.02 and FDR=0) and GS70 (NES=−1.87 and FDR=0.0177). The GSEA results of the 4 combinations were consistent with their respective levels of synergism inhibiting lymphoma growth and survival, phosphorylation of 4EBP1, and translation of c-Myc, therefore further support the notion that the TC combination acts mechanistically through silencing the c-Myc transcription program that is vital for lymphoma cells.

Two of the c-Myc target genes, eIF4B and E2F1 were independently evaluated. FIG. 7F demonstrates that the protein levels of both eIF4B and E2F1 were markedly reduced by the TC combination but not by any single agents or the IB combination in the DLBCL cells LY10 and LY7. As E2F1 was among the most highly suppressed gene by TC, ranked as lowest 7^{th} in its transcript level, the decreased protein level of E2F1 is most likely due to suppression by TC at the transcription level. In comparison, the BRD4 inhibitor JQ-1 does not decrease the mRNA or protein level of E2F1 (Delmore et al., 2011). Collectively, the results above demonstrate that TGR-1202 and carfilzomib in combination potently silence the c-Myc and E2F transcription programs. Furthermore, silencing of c-Myc may be a primary driving force for the synergy of TGR-1202 and carfilzomib, but not a nonspecific effect secondary to the cytotoxicity of the drugs.

### Example 8. TGR-1202 and Carfilzomib in Combination were Highly Active Against Primary Lymphoma Cells but not Toxic to Normal Lymphocytes

Because established cancer cell lines may not faithfully recapitulate the diseases in patients, the effects of PI3K and proteasome inhibitors were investigated in primary lymphoma cells isolated fresh from five patients with the following malignancies, relapsed small lymphocytic lymphoma (SLL), treatment naïve chronic lymphocytic leukemia (CLL), treatment naïve blastoid MCL with p53 deletion, refractory angioimmunoblastic T cell lymphoma (AITL), and refractory acute B Lymphoblastic Leukemia (B-ALL). FIG. 6A demonstrated Cal-101 at 2.5, 5, and 7.5 μM produced only a mild degree of inhibition (20-30%) of the primary SLL cells after 48 hours of treatment. Bortezomib produced 10-80% of inhibition of the SLL cells at the concentrations 2.5, 5, and 7.5 nM. Bortezomib at these same three concentrations was also combined with Cal-101 at 2.5, 5, or 7.5 μM. The dose-effect curves of the combinations were essentially superimposable to the curve of bortezomib, indicating lack of synergy between Cal-101 and bortezomib. FIG. 6B demonstrates that TGR-1202 at 2.5, 5, and 7.5 μM produced mild degrees of inhibition (20-30%) of the primary SLL cells after 48 hours of treatment. Carfilzomib produced 10-90% of inhibition of the CLL cells at the concentrations 2.5, 5, and 7.5 nM. The inhibition curves of the combinations, even when only 2.5 μM TGR-1202 was combined with carfilzomib, separated widely from the curve of carfilzomib as a single agent. The EOB values of the combinations were consistently above 20, indicating potent synergy between these two drugs. FIGS. 6C and 6E demonstrated that Cal-101 did not enhance the cytotoxicity of bortezomib in CLL and MCL cells respectively. In contrast, TGR-1202 even at the low concentration of 2.5 μM markedly increased the activity of carfilzomib in models of CLL and MCL (FIG. 6D & FIG. 6F).

In AITL, Cal-101 was more effective than TGR-1202, and bortezomib more potent than carfilzomib. However, Cal-101 and bortezomib were not synergistic, while TGR-1202+Car remained highly synergistic. In the primary B-ALL cells, the single agents were surprisingly active, but there was no synergy in any of the combinations. Remarkably, in the primary tumor SLL and CLL cells only the combination pair TGR-1202+carfilzomib was observed to potently induce PARP cleavage, inhibit the protein level of c-Myc, and phosphorylation of 4EBP1 (FIGS. 6G & 6H). Neither any of the single agents nor the combination pair Cal-101+bortezomib was able to induce PARP cleavage, downregulate the expression of c-Myc protein, or inhibit the phosphorylation of 4EBP1. Collectively, the results from the these experiments in primary cells suggest that the combination regimen TGR-1202 and carfilzomib may be a promising treatment for aggressive B cell and T cell lymphoma. To determine whether these two drugs will have excessive toxicity to the hematopoietic system, the peripheral blood mononuclear cells, representing primarily lymphocytes, from a healthy donor were treated at four combination conditions. Those conditions were chosen for their known synergy and potent activity in a number of lymphoma models. FIG. 6I demonstrated that PBMC cells were highly resistant to all four combination conditions of TGR-1202+carfilzomib even after 72 hours of treatment, suggesting the combination regimen of TGR-1202 and carfilzomib will be safe for the human hematopoietic system.

### Example 9: TGR-1202 Inhibits Casein Kinase-1ε

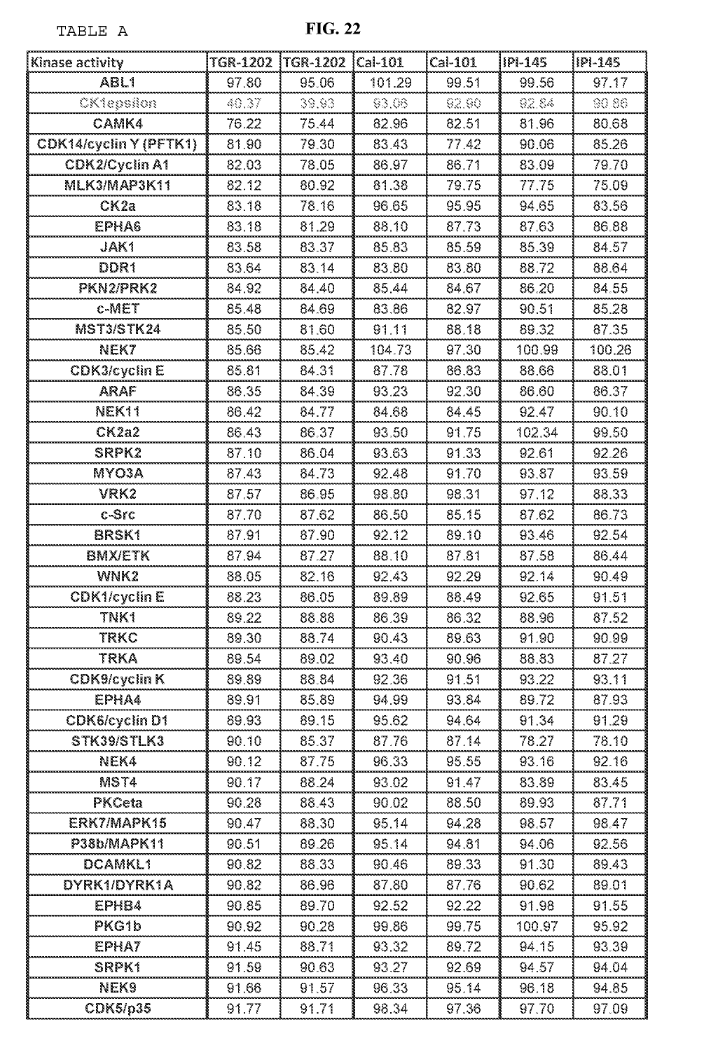

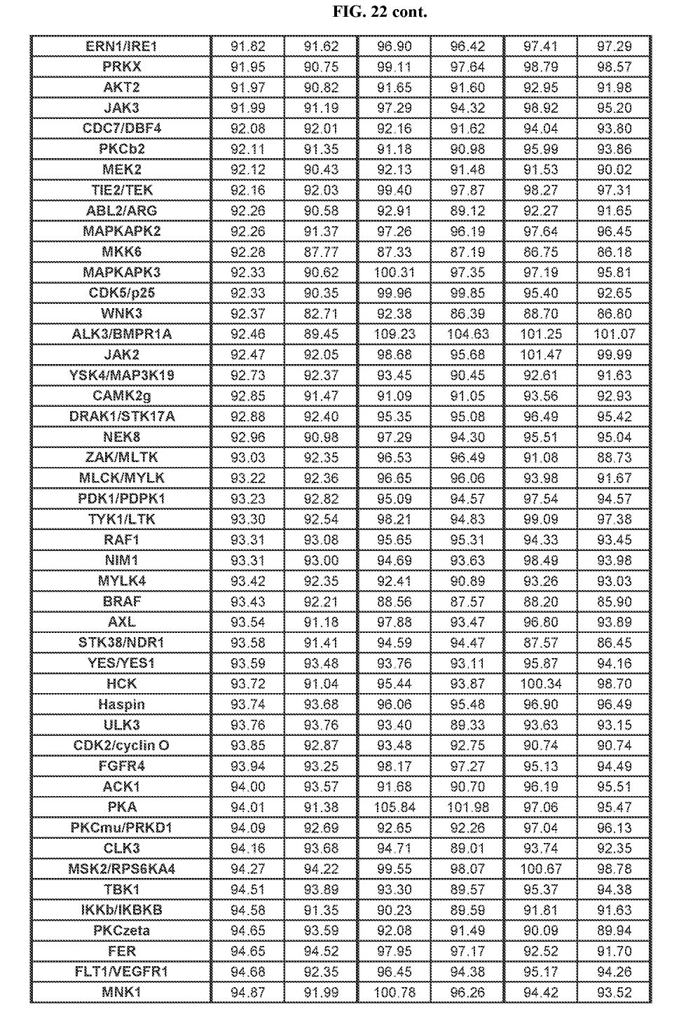

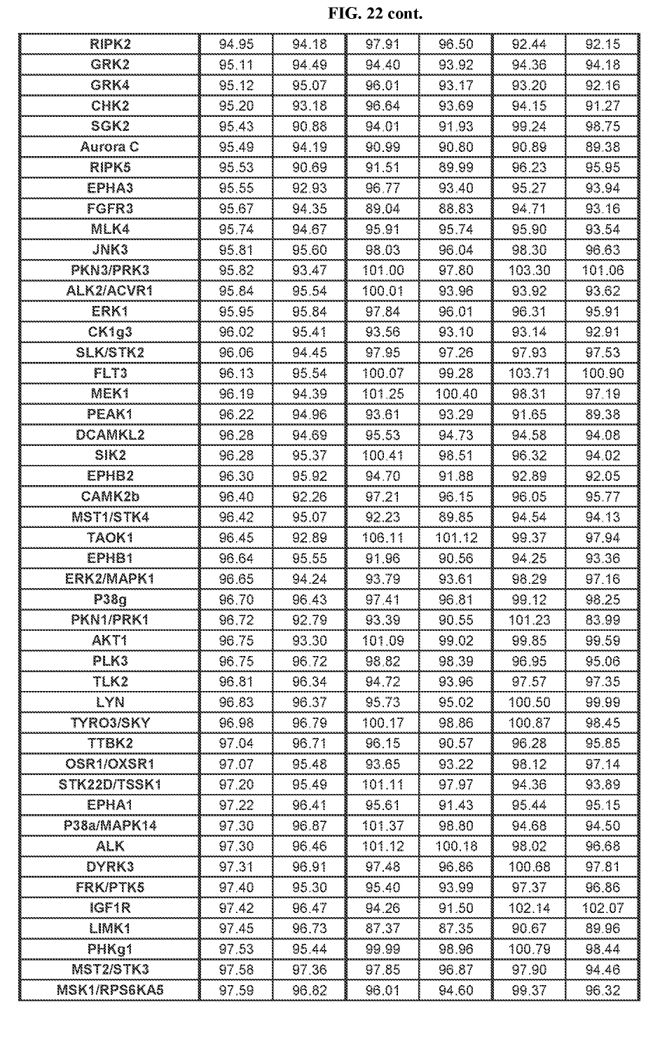

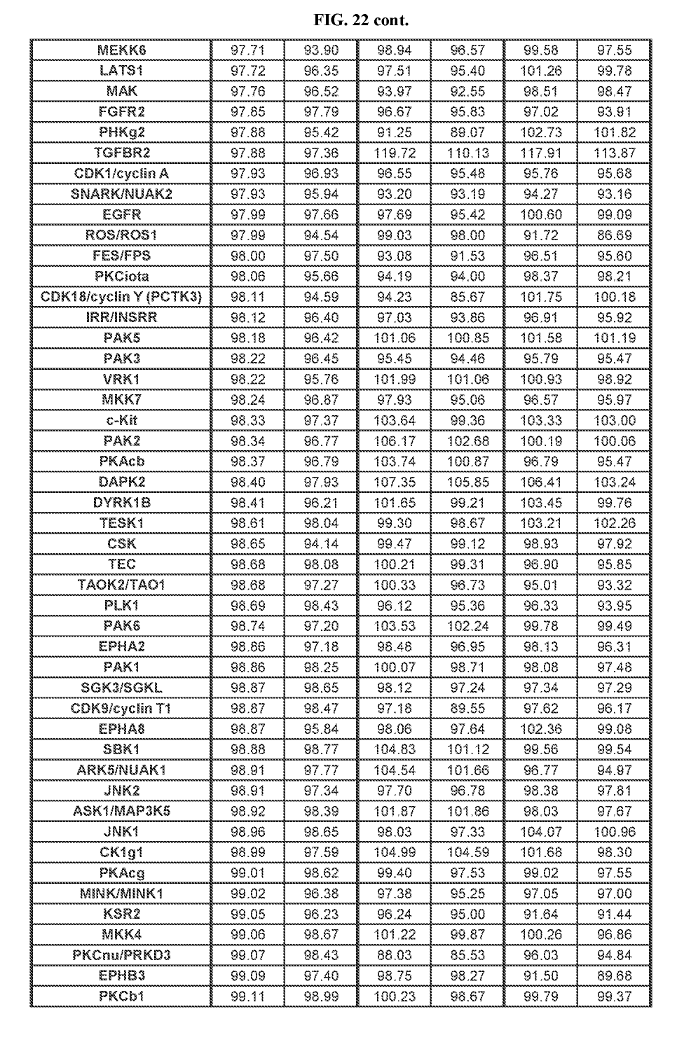

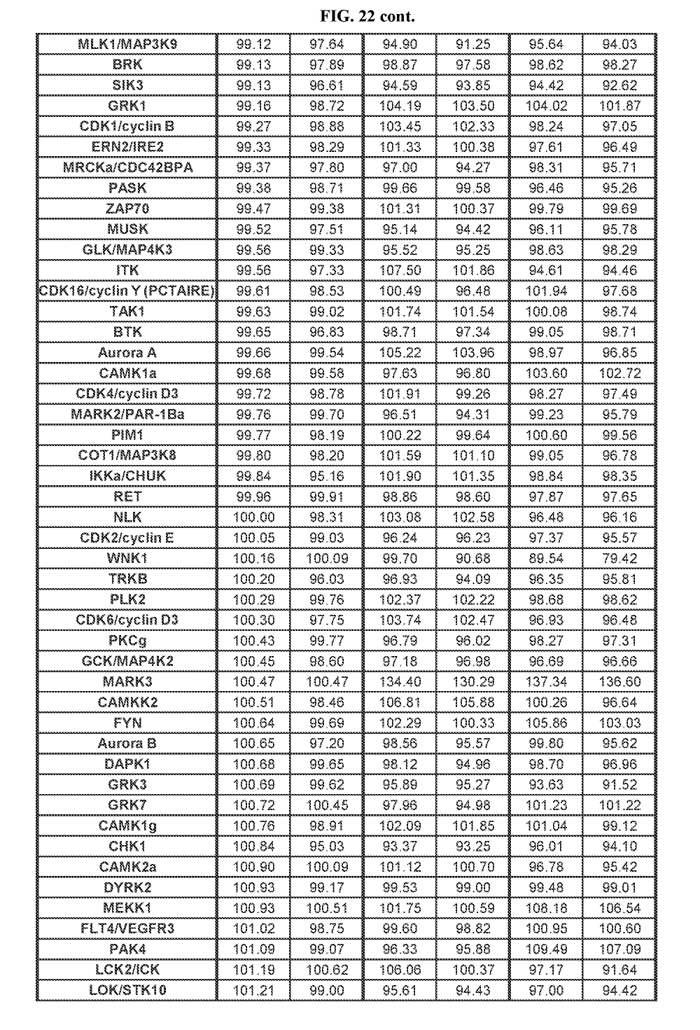

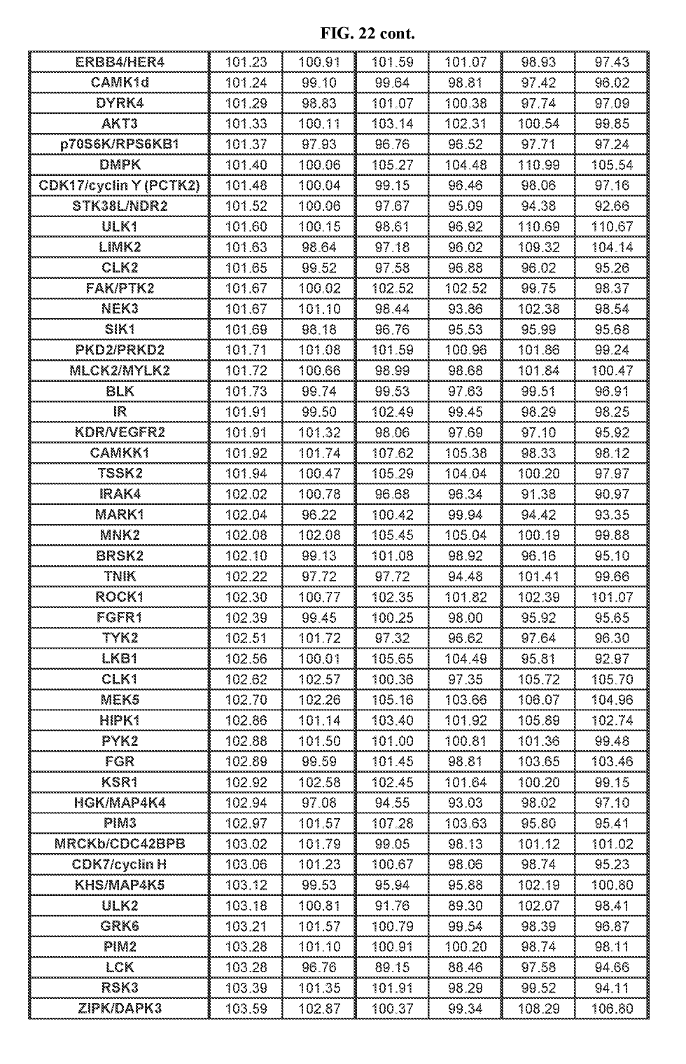

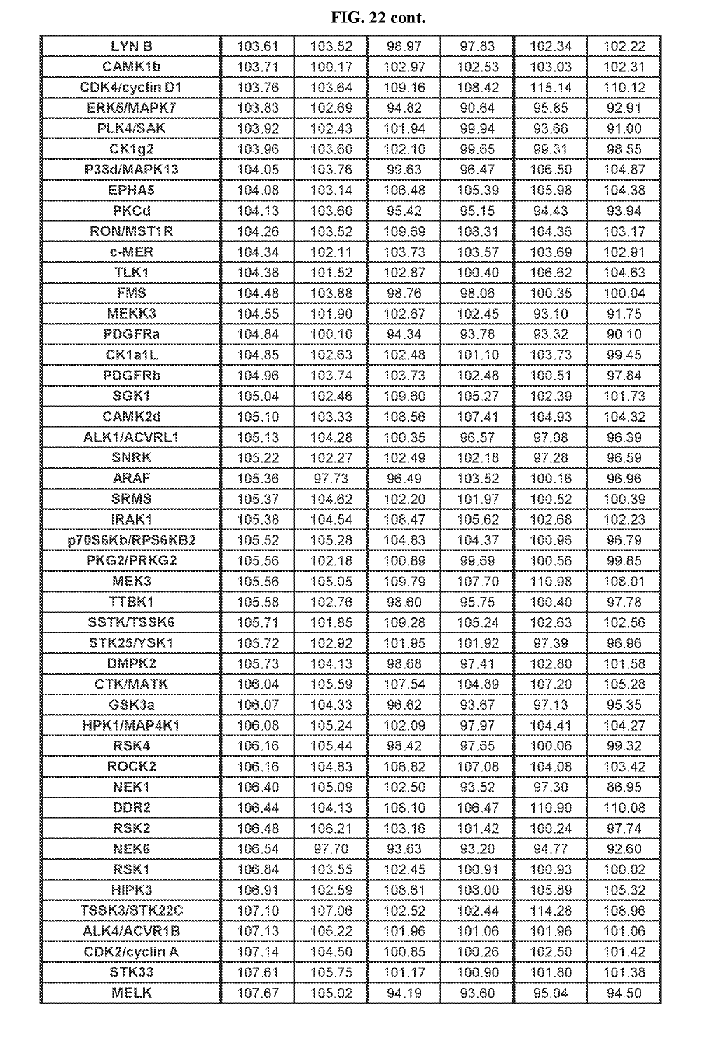

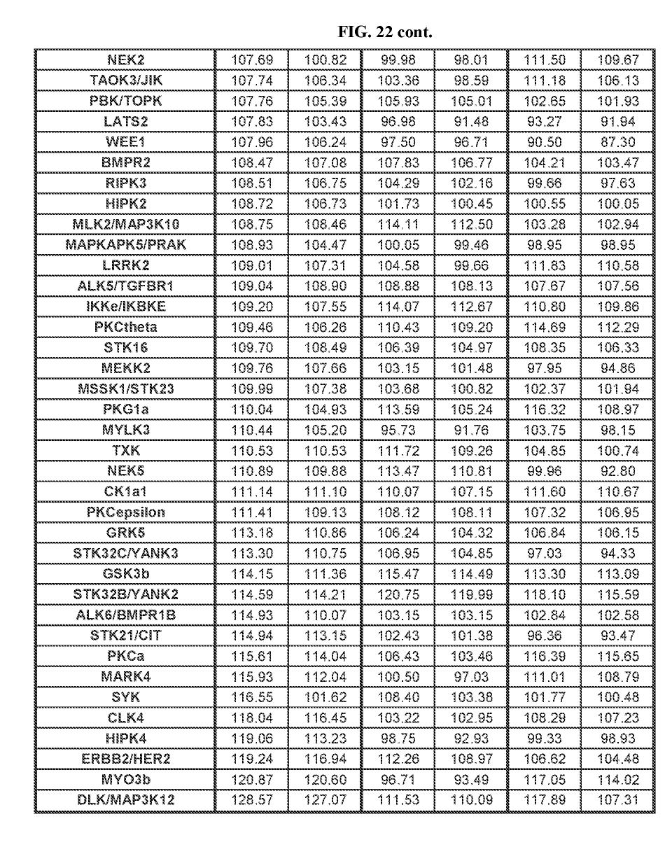

Kinome profiling was performed using the radioisotope filter binding assay from Reaction Biology. The experiment studied the ability of 3 compounds, TGR-1202, CAL-101/idelalisib, and IPI-145/duvelisib to inhibit 359 protein kinases in a cell free system. Each kinase had its own specific substrate according to industry standards. For example, for the kinase CK1 epsilon, the kinase substrate is CK1 peptide with the following sequence [KRRRAL[pS]VASLPGL](Marin, O. et al. Biochem. Biophys. Res. Commun. 198, 898; 1994). Kinase activity was detected by the transfer of 33P-phosphophate from 33P labeled ATP to the phosphorylated amino acid(s) of the substrates. The drugs tested were given a 1 μM. Table A (FIG. 22) described the kinase activity of 359 kinases treated by the 3 PI3K inhibitors. The PI3K inhibitors were not active against this comprehensive panel of protein kinases, with the only exception of CK1 epsilon. Even at the low concentration of 1 μM, TGR-1202 reduced the kinase activity of CK1 epsilon by more than 60%.

### Example 10: Combination of CAL-101/PF-4800567/2/Carfilzomib Decreases c-Myc Expression Comparable to TGR-1202/Cafilzomib Combination

The lymphoma cell line LY10 was treated with PF4800567 (PF), Cal-101 (Cal), TGR-1202 (TG) in combination with the proteasome inhibitor carfilzomib (Cfz) for 48 hours. Cells were collected from each of the treatment groups including the vehicle treated negative control. Viable cells were quantitated by the Cell-Titer Glo assay from Promega. Viable cells in the treated cells were expressed an percentage of the negative control. The results demonstrated that compared to the vehicle treated negative control sample, Cal-101, TGR-1202, carfilzomib, and PF4800567 as single agents reduced the viability to 50-70%. The combination of TGR-1202 and carfilzomib produced potent synergistic inhibition, with a viability of 10%; Cal-101 and carfilzomib were additive, reducing the viability to 30%; Adding PF4800567 to the additive combination of Cal-101 and carfilzomib reduced the viability from 30% to 5%; PF4800567 was synergistic with carfilzomib, producing a viability of only 8%; Adding PF4800567 to the synergistic combination of TGR-1202 and carfilzomib essentially reduced viability to zero. These results thus demonstrated that in the presence of carfilzomib, TGR-1202 was equivalent to the combination of Cal-101/idelalisib and PF4800567 in their ability to inhibit the survival of aggressive lymphoma cells. The results of this experiment are shown in FIG. 8.

### Example 11: PI3K, CK-1δ and Proteasome Inhibition Provide Sustained Inhibition of c-Myc Synthesis

The lymphoma cell line LY10 was treated with PF4800567 (PF), Cal-101 (Cal), TGR-1202 (TG) in combination with the proteasome inhibitor carfilzomib (Cfz) for 12 and 24 hours. Protein extracts were processed for Western blot using antibodies against c-Myc, phosphorylated 4EBP1, total 4EBP1, and beta actin. The results demonstrated that compared to the vehicle treated negative control sample, PF&Cfz and Cal&Cfz produced moderate inhibition of the c-Myc level and phosphorylation of 4EBP1. The combinations PF&Cal&Cfz and TG&Cfz produced marked inhibition of the c-Myc level and phosphorylation of 4EBP1 at 12 hours. At 24 hours, the inhibitory effects of PF&Cfz and Cal&Cfz on c-Myc has warned off significantly, causing the level of c-Myc to rebound. In contrast, PF&Cal&Cfz and TG&Cfz produced even deeper inhibition of the c-Myc level and phosphorylation of 4EBP1 at 24 hours. Therefore, with the presence of carfilzomib, TGR-1202 was equivalent to the combination of Cal-101/idelalisib and PF4800567 in their ability to reduce the protein level of c-Myc and to inhibiting the phosphorylation of 4EBP1. The results of this experiment is shown in FIG. 9.

### Example 12: Overexpression of eIF4E Suppresses the Synergistic Activity of TGR-1202 and Carfilzomib

The results strongly implicate phosphorylation of 4EBP1 as a central mechanistic target of the synergistic combination TC. Because TC potently inhibits phosphorylation of 4EBP1 without affecting the protein level of total 4EBP1 (FIG. 4), dephosphorylated 4EBP1 is expected to efficiently sequester eIF4E leading to repression of mRNA translation. It was hypothesized that overexpression of eIF4E will mitigate the efficacy of TC. FIG. 11A demonstrates that overexpression of eIF4E by a lentivirus protected myeloma cell line H929 from the synergistic combination TC. FIG. 11B demonstrates that without any drug treatment eIF4E overexpression did not cause further accumulation of c-Myc. Upon treatment by the synergistic combination TC, eIF4E overexpression prevented reduction of c-Myc translation caused by the drug combination.

Collectively these data establish that the PI3Kδ inhibitor TGR-1202 and the proteasome inhibitor carfilzomib in combination synergistically silence the translation of c-Myc and c-Myc dependent transcription and survival programs in lymphoma and myeloma cells by potently inhibiting the phosphorylation of 4EBP1. Importantly, similar drug: drug combinations including IC, TB, and IB are much less effective in silencing c-Myc.

### Example 13: TGR-1202 and a Novel Analog CUX-03173 are Structurally Related to the Selective CK1ε Inhibitor PF4800567 and Demonstrate Activity Targeting CK1ε

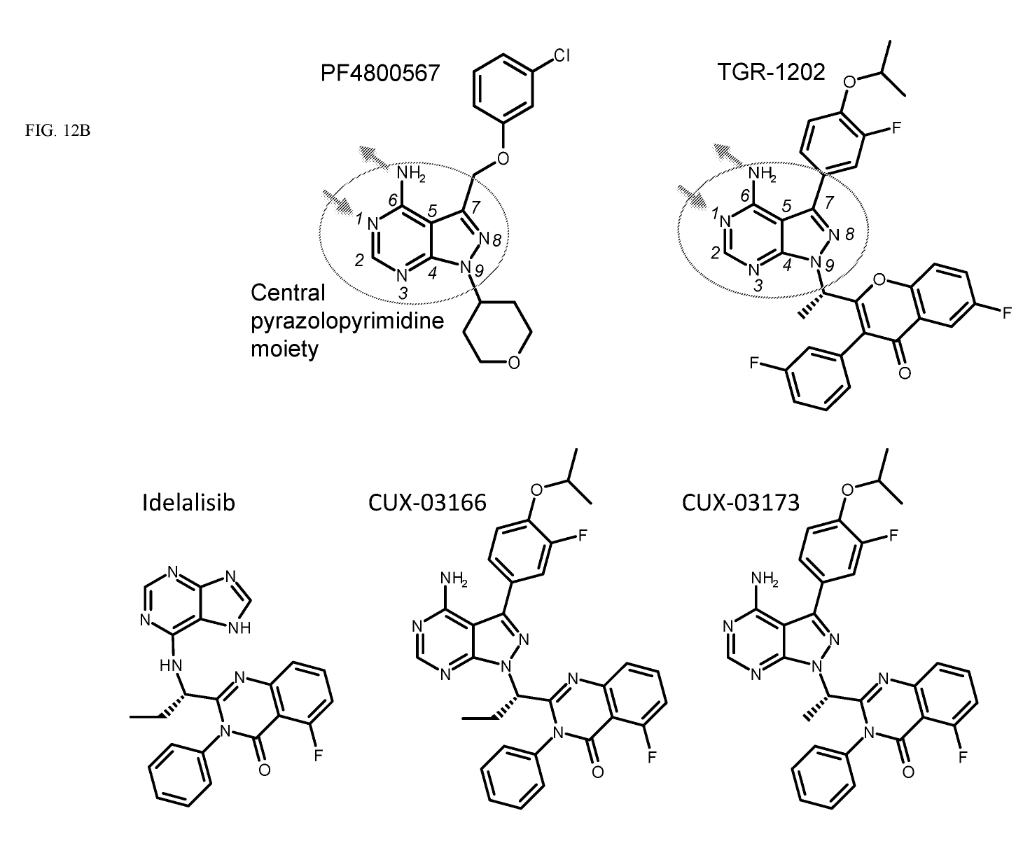

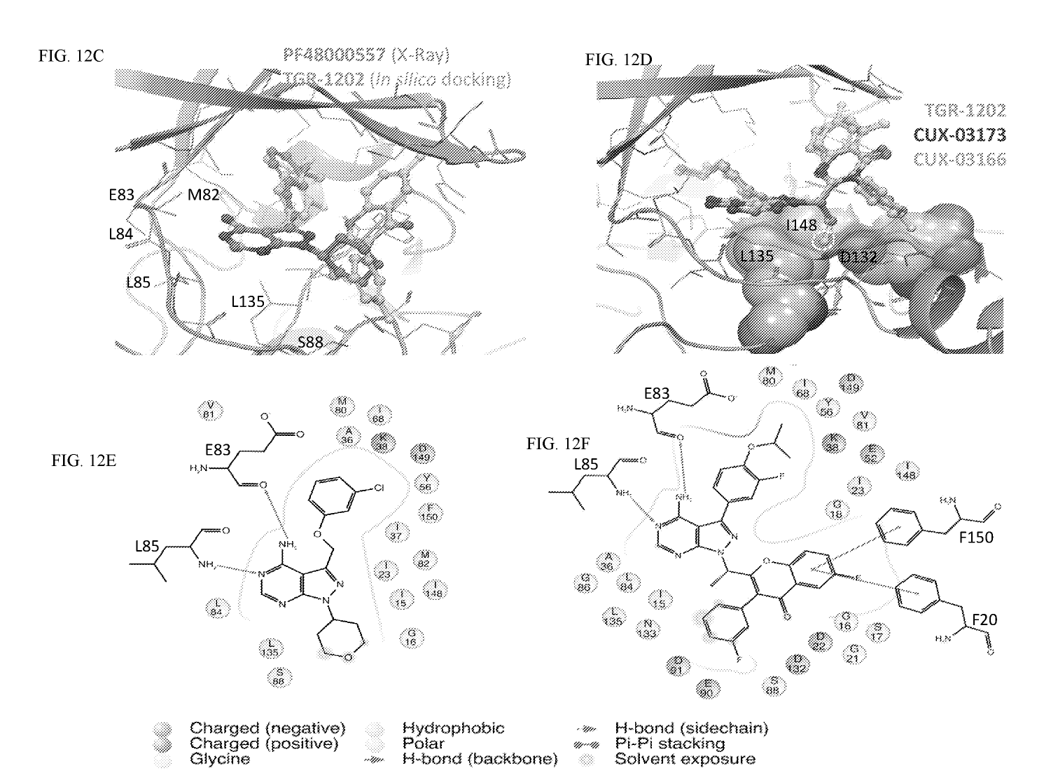

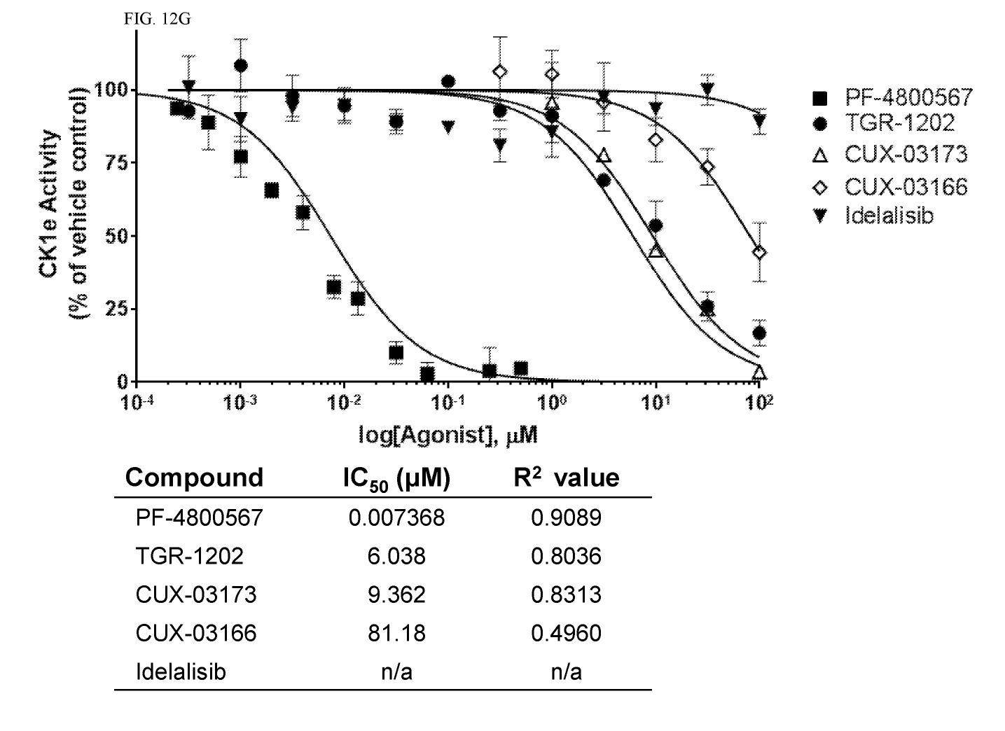

The results have demonstrated that TGR-1202 is superior to idelalisib when combined with proteasome inhibitors. This distinction cannot be explained by their effects on their intended target, i.e. the lipid kinase PI3Kδ since both TGR-1202 and idelalisib selectively and potently inhibit PI3Kδ with EC50 (half maximal effective concentration) values at 24 and 16 nM respectively (Deng et al., 2013). It was hypothesized that TGR-1202 may distinguish itself from idelalisib by targeting additional, and not yet identified, protein kinases. To test this hypothesis, the activity of TGR-1202, idelalisib, and IPI-145/duvelisib was compared on a panel of 365 wild-type protein kinases using the kinome profiling platform from Reaction Biology (Malvern, Pa.). The PI3Kδ inhibitors were not active against this large panel of protein kinases with only one exception: at 1 μM TGR-1202 inhibited 60% of the activity of CK1ε, which was not observed with idelalisib or IPI-145 (FIG. 12A). Of note CK1ε, which is more than 97% identical to CK1ε in their kinase domains, was not inhibited by TGR-1202.

Remarkably, TGR-1202 and the CK1ε selective inhibitor PF4800567 (Long et al., 2012; Walton et al., 2009) are both built around a central pyrazolopyrimidine amine moiety substituted at the same two positions (positions 7 and 9, see FIG. 12B). In contrast, idelalisib, which is not active against CK1ε, does not possess this central pyrazolopyrimidine moiety but a reminiscent adenine ring, which is furthermore only substituted on its amine group (FIG. 12B). To further test the importance of the chemical scaffold built around the pyrazolopyrimidine moiety, shared by PF4800456 and TGR-1202 for CK1ε inhibition, two hybrid compounds where the adenine moiety of Idelalisib was replaced by the top moiety of TGR-1202 were synthesized, including the central pyrazolopyrimidine amine moiety (compounds CUX-03166 and CUX-03173, FIG. 12B).

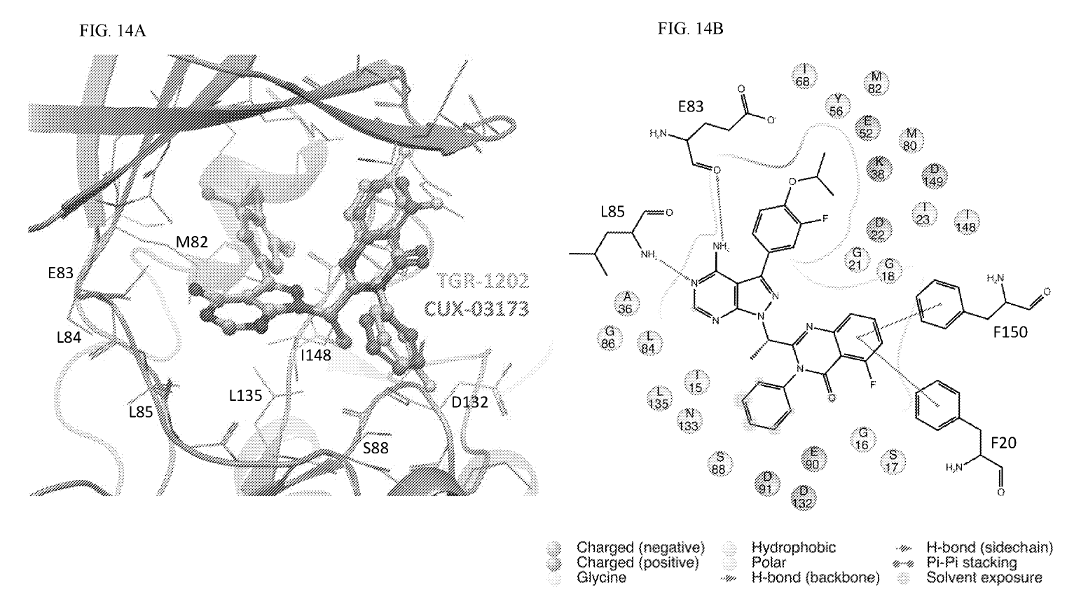

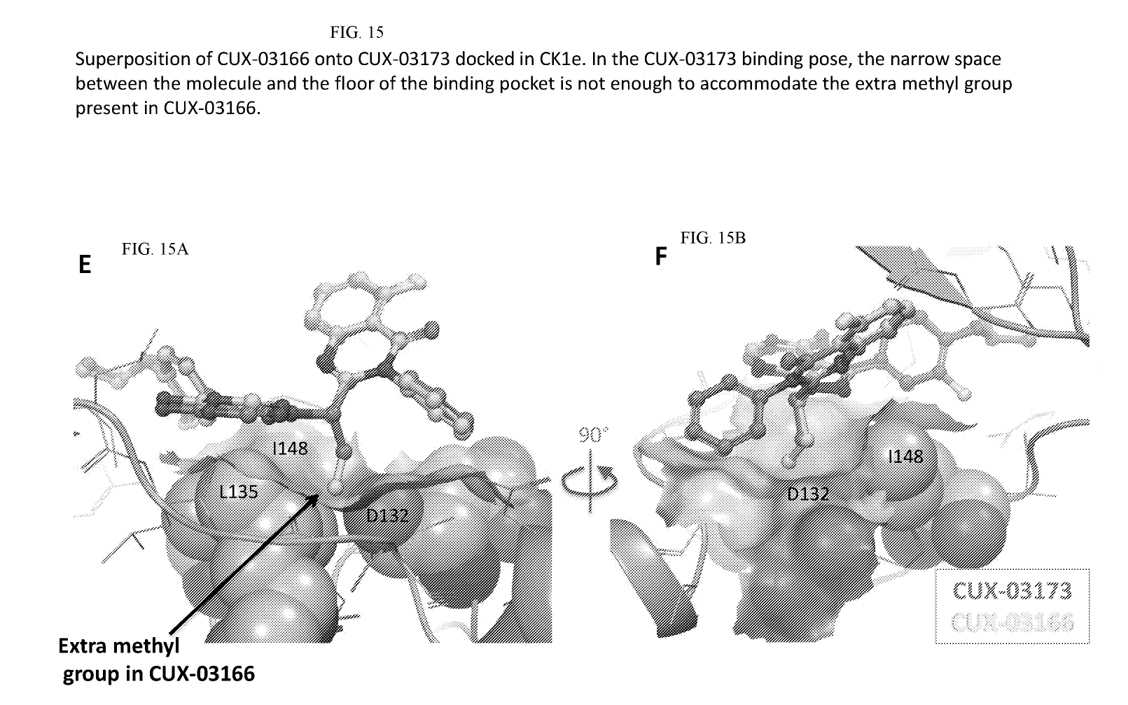

The X-ray crystal structure of PF4800567 bound into the ATP binding pocket of CK1ε (Long et al., 2012) reveals the structural basis of CK1ε inhibition by this compound. In particular, it confirms that the central pyrazolopyrimidine amine moiety plays a key role in the binding of the drug to the target as it establishes two hydrogen bonds with the hinge region of CK1ε (FIG. 12C-FIG. 12F) (Long et al., 2012). In this orientation, the chlorobenzen moiety of PF4800567 (substituted at position 7, FIG. 12B) occupies a hydrophobic pocket deeper in the protein (FIG. 12C & FIG. 7E). In silico docking of TGR-1202 into the ATP pocket of CK1ε resulted in top scoring (best docking score −9.3) binding modes very consistent with that of PF4800567, with the pyrazolopyrimidine amine moiety superposing very well and establishing the exact same hydrogen bonds (FIG. 12C-FIG. 12F). Importantly, the good docking scores obtained for these virtual binding modes show that the hydrophobic pocket reached by the chlorobenzen moiety for PF4800567 can favorably accommodate the somewhat larger corresponding moiety in TGR-1202 (FIG. 12C-FIG. 12F). In contrast, while idelalisib contains an adenine moiety that is reminiscent of the central pyrazolopyrimidine amine moiety shared by PF4800567 and TGR-1202, the potential hydrogen bond donors and acceptors are distributed very differently. Moreover, the amine group, which acts as a hydrogen bond donor in PF4800456 and TGR-1202, is substituted by a large moiety on idelalisib adenine moiety. In fact, superposing the adenine ring onto the pyazolopyrimidine amine moiety of PF4800567 bound to CK1ε results in important steric clashes with the target protein. In good agreement with these differences, the crystal structure of idelalisib in complex with PI3K (4XE0.pdb) shows that while the adenine ring is also involved in two hydrogen bonds with the hinge region of PI3K, it does so in a totally different orientation (Somoza et al., 2015). Superposing the pair of hydrogen bond donor and acceptor of idelalisib in PI3K onto those of PF4800567 bound to CK1ε also results in important steric clashes of idelalisib with CK1ε. Consistently, in silico docking of Idelasilib fails to find high-scoring binding modes (best docking score −3.8) into CK1ε ATP pocket. Overall these results are very consistent with the kinome profiling data showing that TGR-1202 possess CK1ε inhibiting activity while Idelasilib does not. Consistently with its high chemical similarity with TGR-1202, in silico docking of CUX-03173 results in a top-binding pose very close to that of TGR-1202, and with similar docking score (FIG. 14). Interestingly, while CUX-03166 differs from CUX-03173 by only the addition of a methyl group, in silico docking of CUX-03166 in CK1ε results in poses with significantly worst docking scores than CUX-03173 and TGR-1202. The proposed binding pose of CUX-03173 provides a possible structural explanation, as it places the extra methyl of CUX-03166 at a position where the floor of the ATP-binding pocket is very close to the compound and leaves insufficient room for the methyl group (FIG. 12D, and FIG. 15). Finally, this in silico structural study support TGR-1202 and CUX-03173 as potential inhibitors of CK1ε, and idelalisib as inactive against CK1ε. It also suggests that CUX-03166 will likely be less potent than CUX-03173 and TGR-1202 against CK1ε.

Next, the CK1ε inhibiting activity of the above compounds was experimentally determined using the ADP-Glo™ Kinase Assay kit from Promega and recombinant CK1ε expressed by baculovirus in Sf9 insect cells. FIG. 12G demonstrates that PF4800567 was highly potent against CK1ε with an IC50 of 7.4 nM, consistent with previous reports (Walton et al., 2009). TGR-1202 was active against CK1ε, with an IC50 value of 6.0 μM. The IC50 for CUX-03173 was 9.4 μM. In contrast, idelalisib or CUX-03166 did not reach 50% inhibition even at 50 μM. These results demonstrate that among PI3Kδ inhibitors TGR-1202 is uniquely equipped with structural features suitable for targeting CK1ε.

### Example 14: CK1ε Regulates c-Myc Translation in Concert with the PI3Kδ and Proteasome Pathways and is a Target of TGR-1202 in Lymphoma Cells

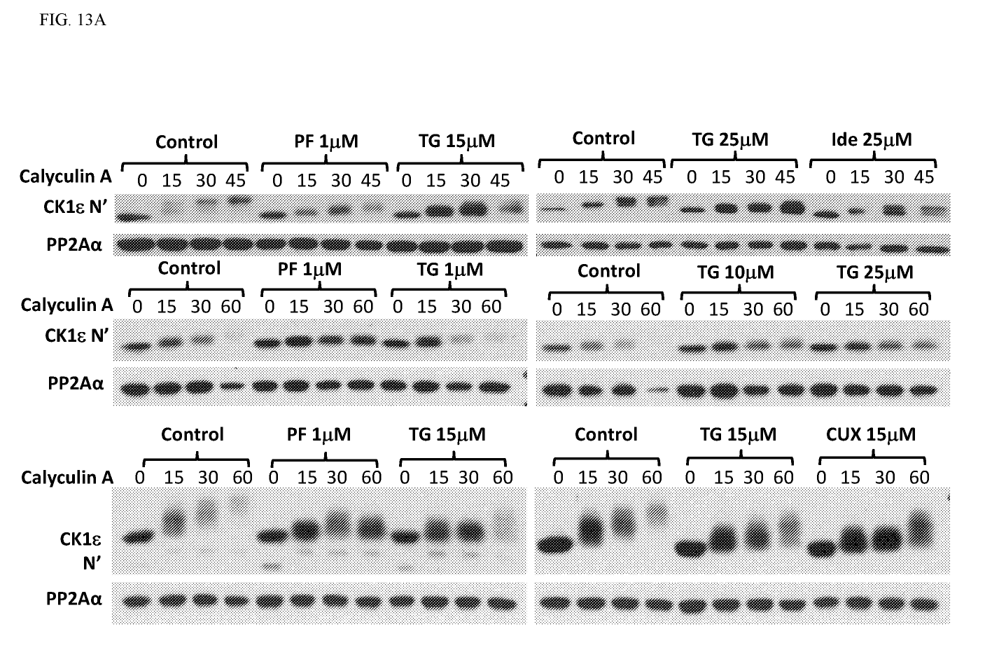

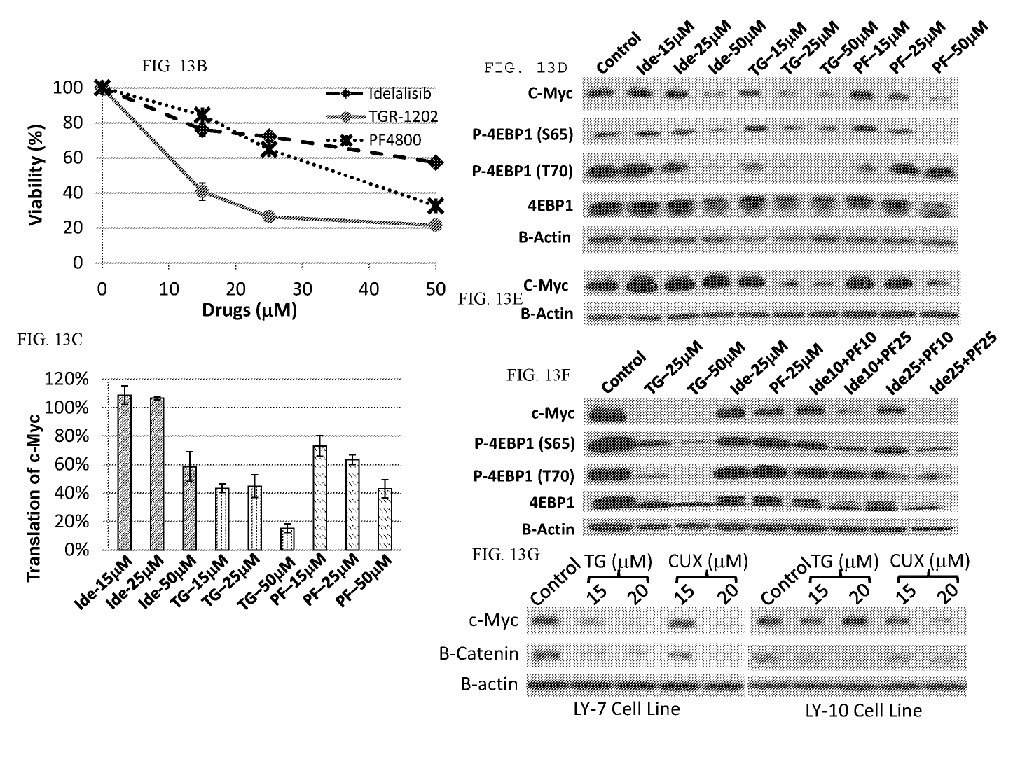

The effects of TGR-1202 on intracellular CK1ε were tested by examining its effect on the autophosphorylation of CK1ε carboxyl-terminus regulatory domain (Cegielska et al., 1998; Cheong et al., 2011; Rivers et al., 1998). Autophosphorylation is continuously reversed by cellular protein phosphatases that are sensitive to the phosphatase inhibitors such as calyculin A. CK1ε inhibitors and phosphatase inhibitors have opposing effects on CK1ε as they stimulate dephosphorylation and phosphorylation of CK1ε respectively. In the negative control not treated by any of the tested drugs, addition of calyculin A produced a time dependent up-shifting of the CK1ε band in the DLBCL cell line LY7 (FIG. 13A). In samples treated by PF4800567 at 1 μM or TGR-1202 at 15-25 μM there was no up-shifting of the CK1ε band. In contrast, in LY7 cells treated by idelalisib at 25 μM the band clearly shifted upward in a manner similar to the negative control. Consistent with the results from the kinase assays CUX-03173 at 15 μM inhibited the autophosphorylation of CK1ε. These results indicate that the PI3Kδ inhibitor TGR-1202 acts as a CK1ε inhibitor in DLBCL cells.

CK1ε has been demonstrated to phosphorylate 4EBP1 and regulate mRNA translation in HEK293 and breast cancer cells (Shin et al., 2014). Based on the pattern of synergy in FIG. 4, for example, it was therefore hypothesized that CK1ε operates as a compensatory pathway to PI3Kε to stimulate c-Myc translation and the survival of lymphoma cells. The hypothesis was first investigated by comparing the potency of idelalisib, PF4800567, and TGR-1202 as single agents in the DLBCL cell line LY7. The pure PI3Kδ inhibitor idelalisib and pure CK1ε inhibitor PF4800567 demonstrated only mild and comparable activity inhibiting the LY7 lymphoma cells in the concentration range of 10-50 μM (FIG. 13B). In contrast, TGR-1202 was significantly more effective than idelalisib and PF4800567 at inhibiting lymphoma cell survival. Similarly, FIG. 13C demonstrates that when measured by inhibition of c-Myc translation, TGR-1202 was most active, followed in decreasing order by PF4800567 and idelalisib. FIG. 13D demonstrates that in the concentrations ranging from 25-50 μM TGR-1202 potently suppressed phosphorylation of 4EBP1 and the protein level of c-Myc with 6 hours of treatment, while such effects were observed for idelalisib and PF4800567 only at the concentration of 50 μM. Interestingly, the expression of c-Myc in lymphoma cells treated by idelalisib and to a less degree by PF4800567 rebounded by 24 h (FIG. 13E). In contrast, the suppression of c-Myc expression by TGR-1202 was not relieved at 24 h. FIG. 13F further demonstrates that at 25 μM TGR-1202 was highly effective at inhibiting the phosphorylation of 4EBP1 and protein level of c-Myc in the DLBCL cell line LY7. Those potent effects caused by 25 μM TGR-1202 were reproduced entirely by combining 25 μM idelalisib with 25 μM PF4800567, partially by combining 10 μM idelalisib with 25 μM PF4800567, and minimally or none at all by combining 10-25 μM idelalisib with 10 μM PF4800567. Finally, FIG. 13G demonstrates that the novel analog of TGR-1202, CUX-03173, was as potent as TGR-1202 in inhibiting the protein level of c-Myc, and both agents inhibited beta-catenin, a target of CK1ε in the LY7 and LY10 cell lines. Collectively these results indicate that TGR-1202 targets both PI3Kδ and CK1ε in lymphoma cells in order to achieve superior activity in silencing c-Myc, which was recapitulated by combining pure PI3Kδ and CK1ε inhibitors.

### Example 15. TGR-1202 is a Selective PI3Kδ Inhibitor Distinct from Idelalisib

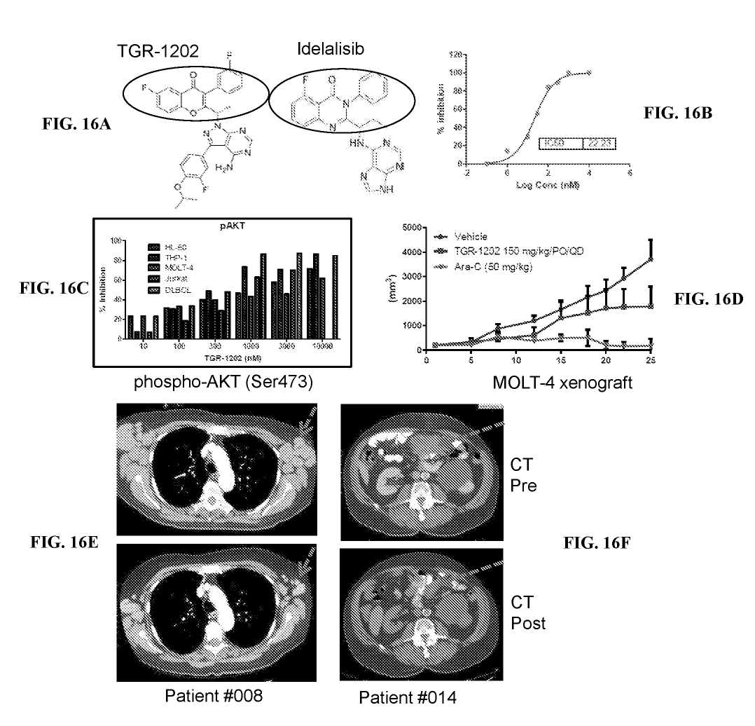

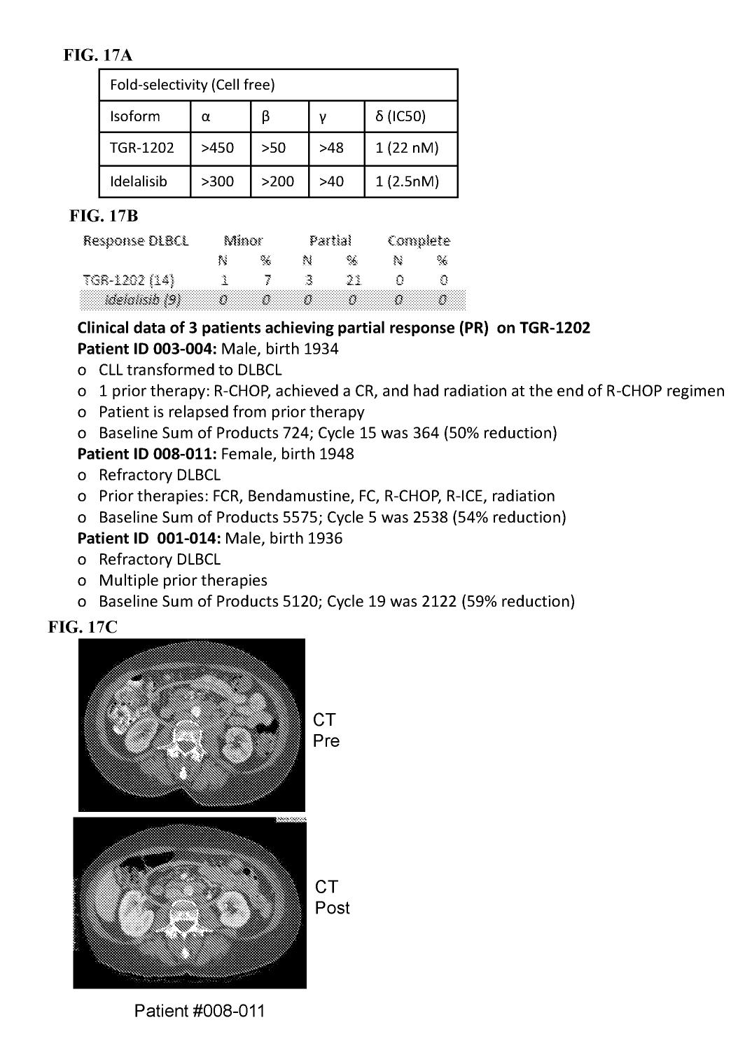

TGR-1202 has the core structure required for targeting PI3K delta (PI3Kδ), as circled in FIG. 16A. Notably, TGR-1202 does not have the nitrogen heterocyclic present in idelalisib, a selective PI3Kδ inhibitor recently approved in the US for the treatment of indolent lymphoma and chronic lymphocytic leukemia (CLL). In a kinase assay based on detection of phosphatidylinositol (3,4,5)-trisphosphate (PIPS), TGR-1202 potently inhibited PI3Kδ with a half maximal inhibitory concentration (IC50) of 22 nanomolar (nM) (FIG. 16A). The IC50 values of TGR-1202 against the other isoforms of PI3K were substantially higher (FIG. 17A), confirming TGR-1202 is a PI3Kδ inhibitor with a selectivity comparable to idelalisib. Next, human lymphoma and leukemia cell lines known for constitutively activated AKT were grown in log phase, then plated into starvation media and treated with TGR-1202 or the vehicle control for 4 hours. TGR-1202 inhibited phosphorylated AKT at Ser473 in a concentration dependent manner (FIG. 16C). At 1 micromolar (μM) TGR-1202 reduced the phosphorylation of AKT by 43-87% in these starved cell lines. In a subcutaneous xenograft model of T-cell acute lymphoblastic leukemia (T-ALL) in NOD/SCID mice using the MOLT-4 cell line, daily oral treatment with TGR-1202 at 150 mg/kg significantly shrank the tumors by day 24 (p<0.001) (FIG. 16D). Finally, in clinical trials TGR-1202 produced a partial response in 3 of 14 patients with DLBCL (FIG. 16E, and FIGS. 17B & C). In contrast, idelalisib did not produce any responses in 9 patients with DLBCL [Westin, J. R., Status of PI3K/Akt/mTOR pathway inhibitors in lymphoma Clin Lymphoma Myeloma Leuk, 2014. 14(5): p. 335-42].

### Example 16. CK1ε Regulates c-Myc Translation Via 4EBP1 in Concert with the PI3Kδ-mTOR Pathway in Lymphoma Cells

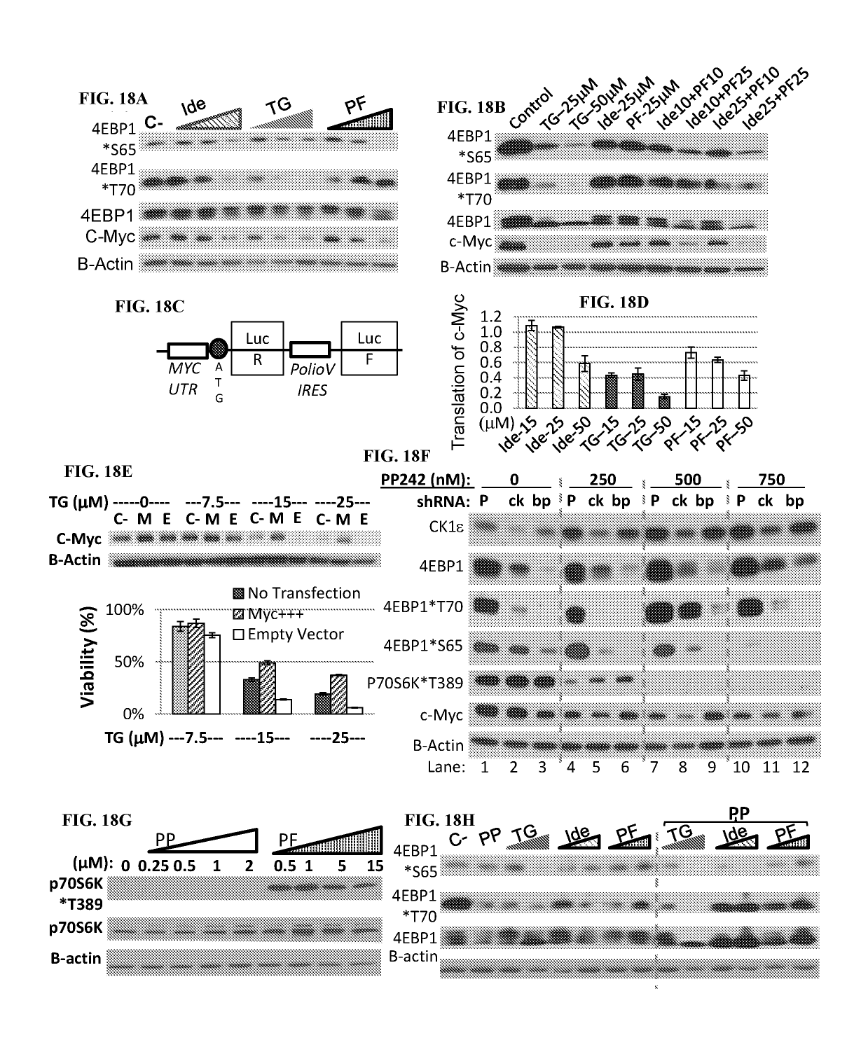

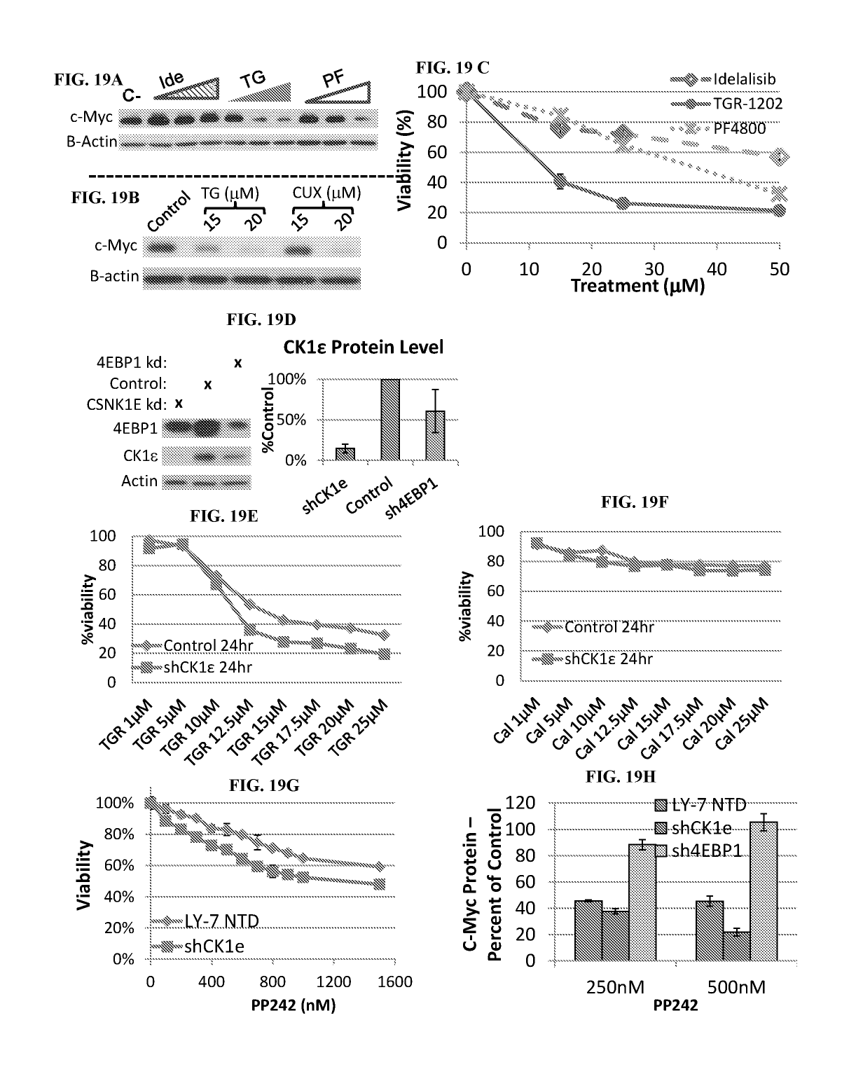

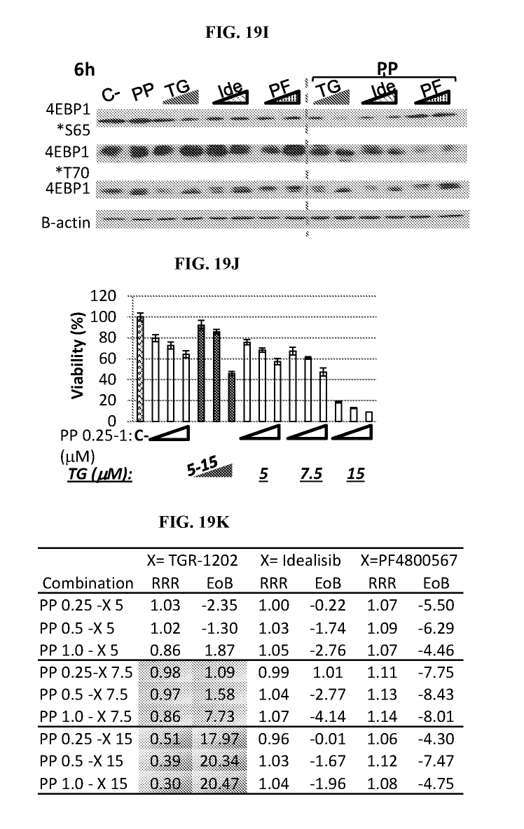

Next, effects were examined of TGR-1202 on 4EBP1, a key translation inhibitor that can be potentially regulated by PI3K, mTOR, and CK1E. FIG. 18A demonstrates that in the concentrations ranging from 15-50 μM TGR-1202 was more potent than idelalisib and PF4800567 at suppressing the phosphorylation of 4EBP1 and the protein level of c-Myc in the DLBCL cell line LY7 treated for 6 h. TGR-1202 and CUX-03173 were more effective than idelalisib and PF4800567 in reducing the protein level of c-Myc at 24 h (FIGS. 19A & B), and TGR-1202 was most potent among these compounds in the cytotoxicity assay in the lymphoma cell line LY7 (FIG. 19C). Combining the selective PI3Kδ inhibitor idelalisib and selective CK1ε inhibitor PF4800567 reproduced the effects of the dual PI3Kδ/CK1 ε inhibitor TGR-1202 on 4EBP1 and c-Myc (FIG. 18B). To determine whether TGR-1202 regulates specifically the translation of c-Myc, a bi-cistronic luciferase reporter as shown in FIG. 18C was designed. Translation of renilla luciferase (LucR) is regulated by the 5′ UTR of c-MYC and is dependent on eIF4F. In contrast, translation of firefly luciferase (LucF) is not eIF4F dependent as it has the Polio virus internal ribosome entry site (IRES). The relative efficiency of cap dependent translation downstream of the 5′ UTR of c-MYC, as measured by the ratio of LucR/LucF, was reduced by 50% by TGR-1202 at 15 μM but not by idelalisib or PF4800567 at 25 μM (FIG. 18D). Importantly, overexpressing c-Myc protein rescued lymphoma cells treated by TGR-1202 (FIG. 18E). These results suggest that the dual PI3Kδ/CK1ε inhibitor TGR-1202 acts through disrupting the eIF4F dependent translation of c-Myc in lymphoma cells.

To further examine the roles of CK1ε in lymphoma, expression of CK1ε was knocked down by more than 80% using shRNA (FIG. 19D). Knockdown of CK1ε produced a modest but consistent increase of sensitivity to TGR-1202 and PP242, a potent mTORC1/mTORC2 inhibitor, but did not affect the response of lymphoma cells to idelalisib (FIG. 19E-G). These results suggest that signaling through both CK1ε and PI3Kδ-mTOR is required for optimal survival of lymphoma cells, and mTOR is not entirely dependent on PI3Kδ. In the DLBCL cell line LY7 knockdown of CK1ε resulted in a moderate decrease in total 4EBP1 protein and a disproportionately large decrease of phosphorylated 4EBP1 at T70, but did not affect the phosphorylation of 4EBP1 at S65, or the protein level of c-Myc (FIG. 18F, Lanes 1-2). As a control, knockdown of 4EBP1 produced a proportionate large decrease in phosphorylated 4EBP1 at T70 and S65, and mildly inhibited the level of c-Myc (FIG. 18F, Lanes 1&3).

In contrast, inhibition of mTORC1 and mTORC2 by PP242 increased the levels of CK1ε had minimal or no effect on total 4EBP1 or phosphorylated 4EBP1 at T70, markedly inhibited phosphorylation of 4EBP1 at S65 (750 nM) and moderately reduced the level of c-Myc (FIG. 18F, Lanes 1, 4, 7, 10 and FIG. 19H). PP242 markedly augmented the inhibitory effects of CK1ε knockdown on phosphorylated 4EBP1 at T70 and S65 while exerting varied effects on total 4EBP1 and c-Myc (FIG. 18F, Lanes 2, 5, 8, 11). These results indicate that CK1ε and mTOR are involved in phosphorylating distinct residues of 4EBP1 and act cooperatively. To further distinguish CK1ε and mTOR, we examined p70S6K1, which is a substrate of mTORC1 and regulates primarily translational elongation. PP242 potently inhibited phosphorylation of p70S6K1 as expected (FIG. 18F, Lanes 1, 4, 7, 10). Knockdown of CK1ε mildly induced phosphorylation of p70S6K1 (FIG. 18F, Lanes 1, 2), and partially rescued the potent inhibition of p70S6K caused by PP242 (FIG. 18F, Lanes 4, 5). In agreement with genetic perturbation, the selective CK1ε inhibitor PF4800567 substantially increased phosphorylation of p70S6K (FIG. 18G), opposite of the effect of PP242. Finally, the dual PI3Kδ/CK1ε inhibitor TGR-1202, but neither idelalisib nor PF4800567, was synergistic with PP242 inhibiting phosphorylation of 4EBP1 at T70 and cell viability (FIG. 18F and FIG. 19I-K). The result further supports a model that CK1ε and PI3K-mTOR are two compensatory pathways upstream of 4EBP1.

### Example 17. TGR-1202 and Carfilzomib Synergistically Kill Blood Cancer Cells Through Potently Disrupting the 4EBP1-eIF4F-c-Myc Axis

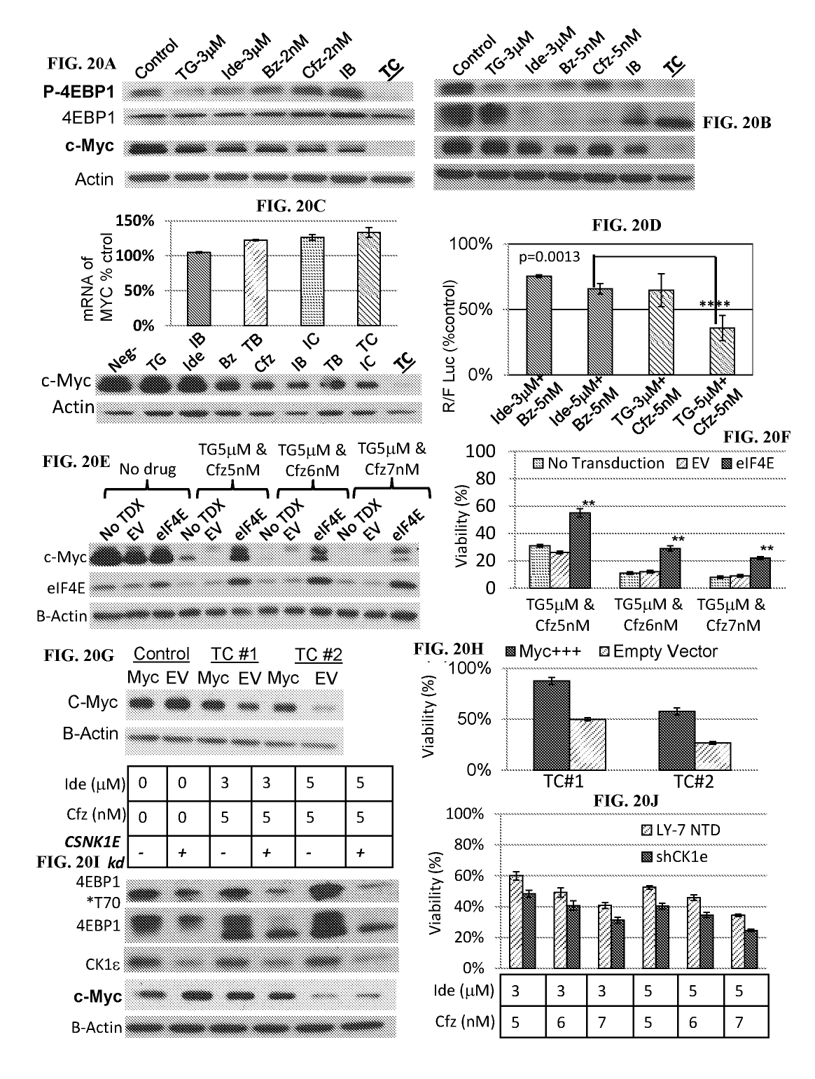

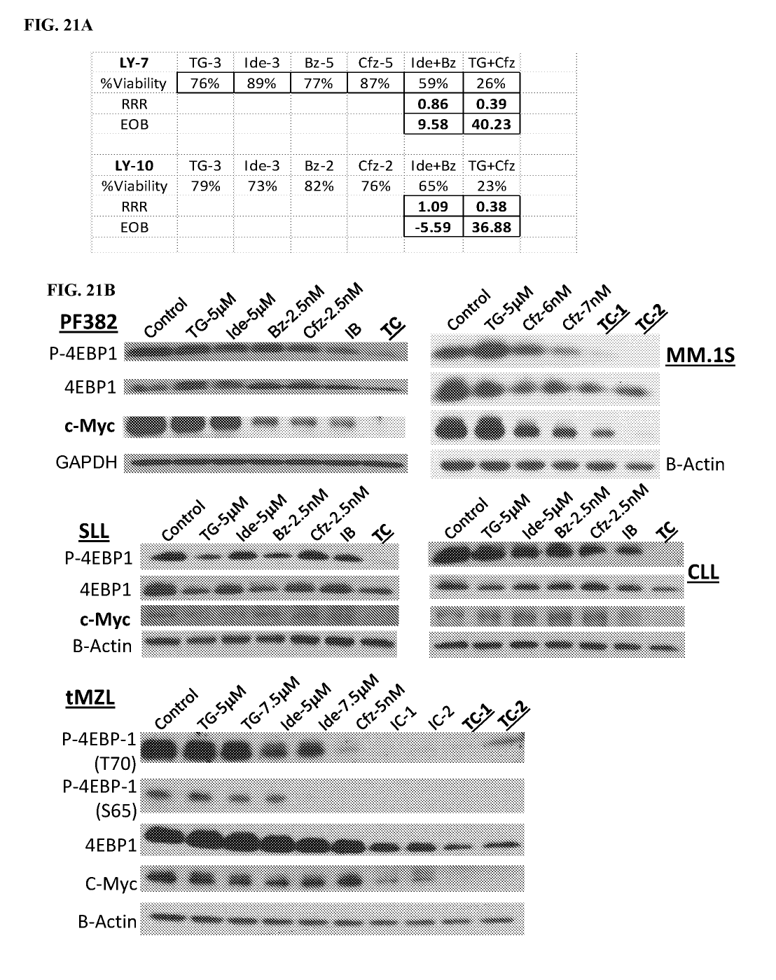

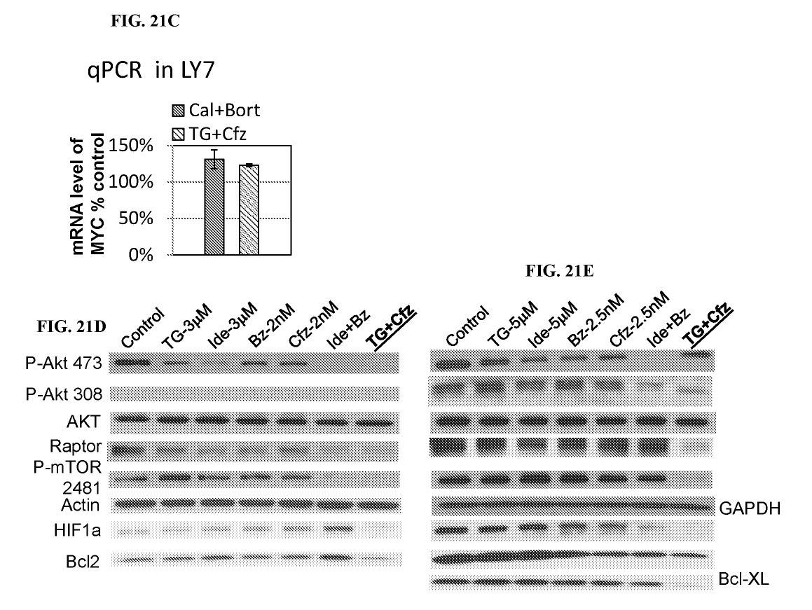

To understand the mechanistic basis of the synergy of TGR-1202 and carfilzomib, it was discovered that the highly synergistic TC combination, but not IB or single agents, potently inhibited phosphorylation of 4EBP1 and the protein level of c-Myc in the LY10 and LY7 cells (FIG. 20A-B and FIG. 21A). Similarly, TC was more effective than IB, IC, and the single agents on 4EBP1 and c-Myc in cell line models of T-ALL (PF382) and MM (MM.1S) as well as primary SLL, CLL, and tMZL cells (FIG. 21B). None of the four combinations, namely IB, TB, IC, and TC decreased the mRNA level of c-Myc (FIG. 20C and FIG. 21C). Using the reporter assay in FIG. 18C, TC was more potent than IB in decreasing the R/F Luc ratio, confirming c-Myc translation as a mechanistic target of TC (FIG. 20D). Moreover, overexpression of eIF4E rescued myeloma cells from the cytotoxicity and suppression of c-Myc level by TC (FIG. 20E-F). In agreement, overexpression of c-Myc also rescued the cytotoxicity of TC in lymphoma cells (FIG. 20G-H). To confirm that CK1ε cooperates with the proteasome and PI3Kδ pathways to regulate 4EBP1, it was demonstrated that knockdown of CK1ε markedly increased the ability of IC (idelalisib and carfilzomib) to inhibit the phosphorylation of 4EBP1 (FIG. 20I). Surprisingly, knockdown of CK1ε did not significantly enhance the cytotoxicity of IC and its suppression of c-Myc protein level (FIG. 20I-J).

As will be apparent to one of ordinary skill in the art from a reading of this disclosure, further embodiments of the present invention can be presented in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. Such equivalents are considered to be within the scope of this invention. Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention. The scope of the invention is as set forth in the appended claims and equivalents thereof, rather than being limited to the examples contained in the foregoing description.

TABLE 1
Partial list of inhibitors of the PI3K-AKT-mTOR signaling pathway

| Product | Description | Company |
|---|---|---|
| Idelalisib | Small molecule | Gilead Sciences Inc.(NASDAQ:GILD) |
| 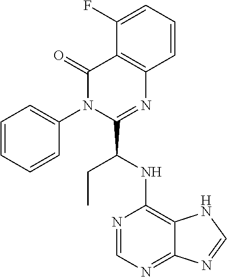 [SMILES: CC[C@H](Nc1ncnc2nc[nH]c12)c1nc2cccc(F)c2c(=O)n1-c1ccccc1] | inhibitor of PI3Kd | |
| IPI-145, Duvelisib | Oral inhibitor of | Takeda Pharmaceutical Co. |
| 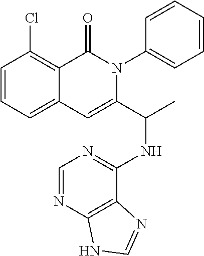 [SMILES: CC(Nc1ncnc2[nH]cnc12)c1cc2cccc(Cl)c2c(=O)n1-c1ccccc1] | PI3Kg and PI3Kd | Ltd.(Tokyo:4502)/Infinity Pharmaceuticals Inc.(NASDAQ:INFI) |
| TGR-1202 | PI3Kd inhibitor | Rhizen Pharmaceuticals S.A./TG |
| | | Therapeutics Inc.(NASDAQ:TGTX) |
| AMG 319 | Small molecule | Amgen Inc. (NASDAQ:AMGN) |
| 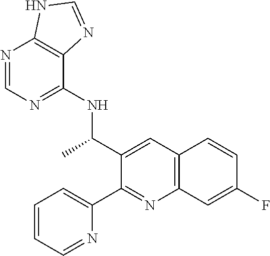 [SMILES: C[C@H](Nc1ncnc2[nH]cnc12)c1cc2ccc(F)cc2nc1-c1ccccn1] | inhibitor of PI3Kd | |
| INCB40093 | PI3Kd inhibitor | Incyte Corp. (NASDAQ:INCY) |
| GS-9820 | PI3Kd inhibitor | Gilead Sciences |
| 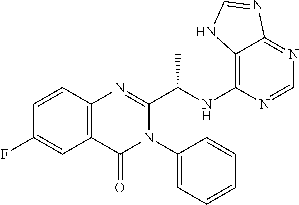 [SMILES: C[C@H](Nc1ncnc2nc[nH]c12)c1nc2ccc(F)cc2c(=O)n1-c1ccccc1] | | |
| RP6530 | Dual PI3Kg and | Rhizen Pharmaceuticals |
| | PI3Kd inhibitor | |
| RP6503 | Dual PI3Kg and | |
| | PI3Kd inhibitor | |
| XL499 | Selective inhibitor | Exelixis Inc.(NASDAQ:EXEL)/Merck & |
| | of PI3Kd | Co. Inc. (NYSE:MRK) |
| PWT143 | PI3Kd inhibitor | Pathway Therapeutics Inc./MEI Pharma |
| | | Inc. (NASDAQ:MEIP) |
| X-339 | Selective inhibitor | Xcovery Holding Co. LLC |
| | of the p110d | |
| | isoform of PI3K | |

Other examples of PI3K inhibitors include but are not limited to: Wortmannin, demethoxyviridin, perifosine, PX-866, IPI-145 (Infinity), BAY 80-6946, BEZ235, MLN1117 (INK1117), Pictilisib, Buparlisib, 5AR245408 (XL147), 5AR245409 (XL765), Palomid 529, Z5TK474, PWT33597, RP6530, CUDC-907, and AEZS-136
Pan-PI3K inhibitors: BEZ235, LY294002, GDC-0941
Selective PI3K inhibitors: BYL719 (alpha); G5K263677 (beta), AS-252424 (gamma)
AKT inhibitors: MK-2206, G5K690693, GDC-0068, A-674563, CCT128930
mTOR inhibitors: AZD8055, INK128, rapamycin
mTORC1 inhibitors: everolimus, temsirolimus, PF-04691502

TABLE 2
CK-1 and PI3K sequences

1) Casein Kinase 1 (epsilon) human protein (SEQ ID NO. 1) NCBI Reference
Sequence: NP_001885

| | |
|---|---|
| 1 | melrvgnkyr lgrkigsgsf gdiylgania sgeevaikle cvktkhpqlh ieskfykmmq |
| 61 | ggvgipsikw cgaegdynvm vmellgpsle dlfnfcsrkf slktvlllad qmisrieyih |
| 121 | sknfihrdvk pdnflmglgk kgnlvyiidf glakkyrdar thqhipyren knltgtarya |
| 181 | sinthlgieq srrddleslg yvlmyfnlgs lpwqglkaat krqkyerise kkmstpievl |
| 241 | ckgypsefst ylnfcrslrf ddkpdysylr qlfrnlfhrq gfsydyvfdw nmlkfgaarn |
| 301 | pedvdrerre hereermgql rgsatralpp gpptgatanr lrsaaepvas tpasriqpag |
| 361 | ntspraisrv drerkvsmrl hrgapanvss sdltgrqevs ripasqtsvp fdhlgk |

2) Casein Kinase 1 (epsilon) human mRNA (SEQ ID NO. 2) NCBI reference
sequence: NM_001894.4

| | |
|---|---|
| 1 | ggttgggatc tgaggggtcc tctctgtgcc catcacagtt tgagcttcag ggaaaagaag |
| 61 | aagaggtctt tgcccttcgt ttttccacgg gaggagaatc aagagtgagc catggagcta |
| 121 | cgtgtgggga acaagtaccg cctgggacgg aagatcggga gcgggtcctt cggagatatc |
| 181 | tacctgggtg ccaacatcgc ctctggtgag gaagtcgcca tcaagctgga gtgtgtgaag |
| 241 | acaaagcacc cccagctgca catcgagagc aagttctaca agatgatgca gggtggcgtg |
| 301 | gggatcccgt ccatcaagtg gtgcggagct gagggcgact acaacgtgat ggtcatggag |
| 361 | ctgctggggc ctagcctcga ggacctgttc aacttctgtt cccgcaaatt cagcctcaag |
| 421 | acggtgctgc tcttggccga ccagatgatc agccgcatcg agtatatcca ctccaagaac |
| 481 | ttcatccacc gggacgtcaa gcccgacaac ttcctcatgg ggctggggaa gaagggcaac |
| 541 | ctggtctaca tcatcgactt cggcctggcc aagaagtacc gggacgcccg cacccaccag |
| 601 | cacattccct accgggaaaa caagaacctg accggcacgg cccgctacgc ttccatcaac |
| 661 | acgcacctgg gcattgagca aagccgtcga gatgacctgg agagcctggg ctacgtgctc |
| 721 | atgtacttca acctgggctc cctgccctgg caggggctca aagcagccac caagcgccag |
| 781 | aagtatgaac ggatcagcga gaagaagatg tcaacgccca tcgaggtcct ctgcaaaggc |
| 841 | tatccctccg aattctcaac atacctcaac ttctgccgct ccctgcggtt tgacgacaag |
| 901 | cccgactact cttacctacg tcagctcttc cgcaacctct tccaccggca gggcttctcc |
| 961 | tatgactacg tctttgactg gaacatgctg aaattcggtg cagcccggaa tcccgaggat |
| 1021 | gtggaccggg agcggcgaga acacgaacgc gaggagagga tggggcagct acgggggtcc |
| 1081 | gcgacccgag ccctgccccc tggcccaccc acgggggcca ctgccaaccg gctccgcagt |
| 1141 | gccgccgagc ccgtggcttc cacgccagcc tcccgcatcc agccggctgg caatacttct |
| 1201 | cccagagcga tctcgcgggt cgaccgggag aggaaggtga gtatgaggct gcacaggggt |
| 1261 | gcgcccgcca acgtctcctc ctcagacctc actgggcggc aagaggtctc ccggatccca |
| 1321 | gcctcacaga caagtgtgcc atttgaccat ctcgggaagt gaggagagcc cccattggac |
| 1381 | cagtgtttgc ttagtgtctt cactgtattt tctttaaaaa aaaaaaaaaa aaaaaaaagg |
| 1441 | caaaaataaa ccactcaaaa gaacaacaaa aaaacccagc acaaaaccga cgatggagtt |
| 1501 | tgtttctttg atttctttgc caatggcaag aagatgagat gccctcagca ctgaggattc |
| 1561 | ttgccccctt gtggtgcccg ctgcccccaa ccttcaggct gccagatgct cccctgacaa |
| 1621 | caccaggcta caggagccag acgccagggc ctgcccggcc tcctgttcct gcccccaccc |
| 1681 | accacctgcc tggagaggaa cgggtcgggt ccgtgtcgga gaagtgacag gtcccagagc |
| 1741 | caaagccggc cctcaagcat catcagggag tggtgtagtc agttgaaggc agttcccacc |
| 1801 | gagttttccg agcctcagaa tccaggagat acgcacagcc ccacccactc tgagatgaca |
| 1861 | gtggctgact tcccgtgctg ggcttttcca ttgtccccct ggcctccagg ctcctcctct |
| 1921 | gcctctccat ggagtgggtg gggaggtggt gggggccggc gtcccctgcg tgtgtgtgtg |
| 1981 | tgtgtgtgtg tgtggatgta ttgacctgtg tttcccaaga cagcaggtgc cacggcccgc |
| 2041 | cccgcctgcc agcccgaatt cccgttctcc tgtgtctact aacaaggaca tgggggtggg |
| 2101 | cggtgacctc cgcatccctc agagctcaga gggtcctcgc tgccaccggt ccccccctag |
| 2161 | cccgtcatca gccggtggca gctccatctt ccattcctgg ttttagggca gaatccatgg |
| 2221 | agactgcttc cagaaggcat ctggctctga gttataaatt acttccctgg tcctgacagt |
| 2281 | cacctggggt cccccctctc cctggttcca cctttctgag gaggagcctg gagtcagggc |
| 2341 | tgggttttgg attaacccat ccttcctagt taacaccttt ttgtttttat tttattttat |
| 2401 | ttttgtttgt tttctccgtg tgtgtgtttt cctaatttat ttacctctgt ttcccctttt |
| 2461 | tccttttttt ttttaattaa agagcaaagc tttttattac tttgtaattt aaaaaactga |
| 2521 | aaaaaaaaaa actgaagaac tttgggggga attttgtact tttttcctgt gtaaatattg |
| 2581 | gacttttttg agctttatcg tggttgttaa tttgaagtaa taaagtagaa aagataaagt |
| 2641 | gaaaaaaaaa aaaaaaaaaa aaaaaaaaaa |

3) phosphatidylinositol 4,5-bisphosphate 3-kinase catalytic subunit delta isoform, or
PI3Kδ, human protein (SEQ ID NO. 3) NCBI Reference Sequence NP_005017.3

| | |
|---|---|
| 1 | mppgvdcpme fwtkeenqsv vvdfllptgv ylnfpvsrna nlstikqllw hraqyeplfh |
| 61 | mlsgpeayvf tcinqtaeqq eledeqrrlc dvqpflpvlr lvaregdrvk klinsqisll |
| 121 | igkglhefds lcdpevndfr akmcqfceea aarrqqlgwe awlqysfplq lepsaqtwgp |
| 181 | gtlrlpnral lvnvkfegse esftfqvstk dvplalmaca lrkkatvfrq plveqpedyt |
| 241 | lqvngrheyl ygsyplcqfq yicsclhsgl tphltmvhss silamrdeqs npapqvqkpr |
| 301 | akpppipakk pssyslwsle qpfrieliqg skvnadermk lvvqaglfhg nemlcktvss |
| 361 | sevsvcsepv wkqrlefdin icdlprmarl cfalyaviek akkarstkkk skkadcpiaw |
| 421 | anlmlfdykd qlktgercly mwpsvpdekg ellnptgtvr snpntdsaaa lliclpevap |
| 481 | hpvyypalek ilelgrhsec vhvteeeqlq lreilerrgs gelyehekdl vwklrhevqe |
| 541 | hfpealarll lvtkwnkhed vaqmlyllcs wpelpvlsal elldfsfpdc hvgsfaiksl |
| 601 | rkltddelfq yllqlvqvlk yesyldcelt kflldralan rkighflfwh lrsemhvpsv |
| 661 | alrfglilea ycrgsthhmk vlmkqgeals klkalndfvk lssqktpkpq tkelmhlcmr |
| 721 | qeaylealsh lqspldpstl laevcveqct fmdskmkplw imysneeags ggsvgiifkn |
| 781 | gddlrqdmlt lqmiqlmdvl wkqegldlrm tpygclptgd rtglievvlr sdtianiqln |
| 841 | ksnmaataaf nkdallnwlk sknpgealdr aieeftlsca gycvatyvlg igdrhsdnim |
| 901 | iresgqlfhi dfghflgnfk tkfginrerv pfiltydfvh viqqgktnns ekferfrgyc |
| 961 | eraytilrrh gllflhlfal mraaglpels cskdiqylkd slalgkteee alkhfrvkfn |
| 1021 | ealreswktk vnwlahnvsk dnrq |

4) PI3Kδ human mRNA sequence: NCBI reference no. NM_005026.3

TABLE 3
Casein Kinase inhibitors
Product Name/Activity
CKI 7 dihydrochloride-CK1 inhibitor

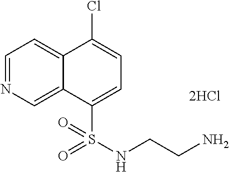 [SMILES: NCCNS(=O)(=O)c1ccc(Cl)c2ccncc12]

(R)-CR8-Dual cdk/CK1 inhibitor

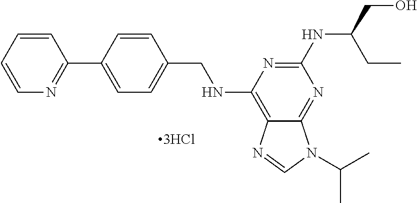 [SMILES: CC[C@H](CO)Nc1nc(NCc2ccc(-c3ccccn3)cc2)c2ncn(C(C)C)c2n1 ; "·3HCl"]

D 4476-Selective CK1 inhibitor. Also inhibits TGF-βRI

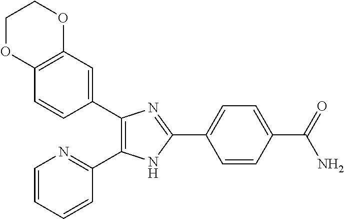 [SMILES: NC(=O)c1ccc(-c2nc(-c3ccc4c(c3)OCCO4)c(-c3ccccn3)[nH]2)cc1]

(R)-DRF053 dihydrochloride-Dual CK1/cdk inhibitor

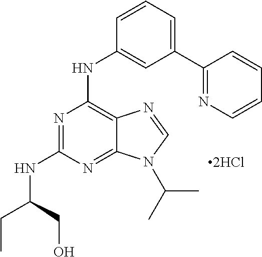 [SMILES: CC[C@H](CO)Nc1nc(Nc2cccc(-c3ccccn3)c2)c2ncn(C(C)C)c2n1]

PF 4800567 hydrochloride-Selective casein kinase
1ϵ inhibitor

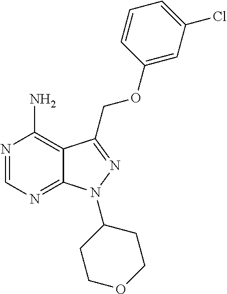 [SMILES: Nc1ncnc2c1c(COc1cccc(Cl)c1)nn2C1CCOCC1]

PF 670462-Potent and selective CK1ϵ and
CK1δ inhibitor

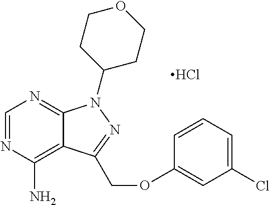 [SMILES: Nc1ncnc2c1c(COc1cccc(Cl)c1)nn2C1CCOCC1 ; Cl]

TA 01-CK1ϵ and CK1δ inhibitor; also inhibits p38α

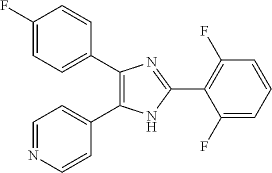 [SMILES: Fc1ccc(-c2nc(-c3c(F)cccc3F)[nH]c2-c2ccncc2)cc1]

TA 02-CK1ϵ and CK1δ inhibitor; also inhibits p38α

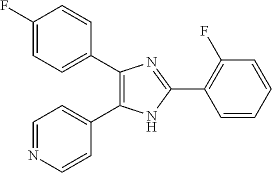 [SMILES: Fc1ccc(-c2nc(-c3ccccc3F)[nH]c2-c2ccncc2)cc1]

TAK 715-Inhibitor of Wnt/β-catenin signaling; cross-
reacts with CK1δ/ϵ

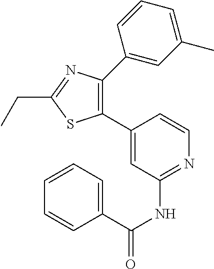 [SMILES: CCc1nc(-c2cccc(C)c2)c(-c2ccnc(NC(=O)c3ccccc3)c2)s1]

LH 846-CK1 delta

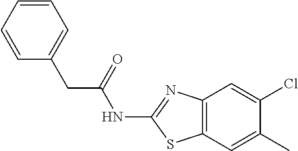 [SMILES: Cc1cc2sc(NC(=O)Cc3ccccc3)nc2cc1Cl]

Lenalidomide-CK1 alpha

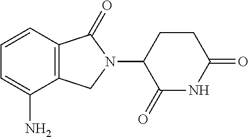 [SMILES: Nc1cccc2c1CN(C1CCC(=O)NC1=O)C2=O]

TABLE 4
Partial list of adjunct chemotherapeutic agents, excluding proteasome
inhibitors, that can be combined with the lead-in c-Myc-silencing
treatments using dual inhibition of PI3K and CK1.

Abiraterone Acetate
Abitrexate (Methotrexate)
Abraxane (Paclitaxel Albumin-stabilized Nanoparticle Formulation)
ABVD
ABVE
ABVE-PC
AC
AC-T
Adcetris (Brentuximab Vedotin)
ADE
Ado-Trastuzumab Emtansine
Adriamycin (Doxorubicin Hydrochloride)
Adrucil (Fluorouracil)
Afatinib Dimaleate
Afinitor (Everolimus)
Aldara (Imiquimod)
Aldesleukin
Alemtuzumab
Alimta (Pemetrexed Disodium)
Aloxi (Palonosetron Hydrochloride)
Ambochlorin (Chlorambucil)
Amboclorin (Chlorambucil)
Aminolevulinic Acid
Anastrozole
Aprepitant
Aredia (Pamidronate Disodium)
Arimidex (Anastrozole)
Aromasin (Exemestane)
Arranon (Nelarabine)
Arsenic Trioxide
Arzerra (Ofatumumab)
Asparaginase Erwinia chrysanthemi
Avastin (Bevacizumab)
Axitinib
Azacitidine
BEACOPP
Becenum (Carmustine)
Beleodaq (Belinostat)
Belinostat
Bendamustine Hydrochloride
BEP
Bevacizumab
Bexarotene
Bexxar (Tositumomab and I 131 Iodine Tositumomab)
Bicalutamide
BiCNU (Carmustine)
Bleomycin
Blinatumomab
Blincyto (Blinatumomab)
Bortezomib
Bosulif (Bosutinib)
Bosutinib
Brentuximab Vedotin
Busulfan
Busulfex (Busulfan)
Cabazitaxel
Cabozantinib-S-Malate
CAF
Campath (Alemtuzumab)
Camptosar (Irinotecan Hydrochloride)
Capecitabine
CAPOX
Carboplatin
CARBOPLATIN-TAXOL
Carfilzomib
Carmubris (Carmustine)
Carmustine
Carmustine Implant
Casodex (Bicalutamide)
CeeNU (Lomustine)
Ceritinib
Cerubidine (Daunorubicin Hydrochloride)
Cervarix (Recombinant HPV Bivalent Vaccine)
Cetuximab
Chlorambucil
CHLORAMBUCIL-PREDNISONE
CHOP
Cisplatin
Clafen (Cyclophosphamide)
Clofarabine
Clofarex (Clofarabine)
Clolar (Clofarabine)
CMF
Cometriq (Cabozantinib-S-Malate)
COPP
COPP-ABV
Cosmegen (Dactinomycin)
Crizotinib
CVP
Cyclophosphamide
Cyfos (Ifosfamide)
Cyramza (Ramucirumab)
Cytarabine
Cytarabine, Liposomal
Cytosar-U (Cytarabine)
Cytoxan (Cyclophosphamide)
Dabrafenib
Dacarbazine
Dacogen (Decitabine)
Dactinomycin
Dasatinib
Daunorubicin Hydrochloride
Decitabine
Degarelix
Denileukin Diftitox
Denosumab
Dinutuximab
DepoCyt (Liposomal Cytarabine)
DepoFoam (Liposomal Cytarabine)
Dexrazoxane Hydrochloride
Docetaxel
Doxil (Doxorubicin Hydrochloride Liposome)
Doxorubicin Hydrochloride
Doxorubicin Hydrochloride Liposome
Dox-SL (Doxorubicin Hydrochloride Liposome)
DTIC-Dome (Dacarbazine)
Efudex (Fluorouracil)
Elitek (Rasburicase)
Ellence (Epirubicin Hydrochloride)
Eloxatin (Oxaliplatin)
Eltrombopag Olamine
Emend (Aprepitant)
Enzalutamide
Epirubicin Hydrochloride
EPOCH
Erbitux (Cetuximab)
Eribulin Mesylate
Erivedge (Vismodegib)
Erlotinib Hydrochloride
Erwinaze (Asparaginase Erwinia chrysanthemi)
Etopophos (Etoposide Phosphate)
Etoposide
Etoposide Phosphate
Evacet (Doxorubicin Hydrochloride Liposome)
Everolimus
Evista (Raloxifene Hydrochloride)
Exemestane
Fareston (Toremifene)
Farydak (Panobinostat)
Faslodex (Fulvestrant)
FEC
Femara (Letrozole)
Filgrastim
Fludara (Fludarabine Phosphate)
Fludarabine Phosphate
Fluoroplex (Fluorouracil)
Fluorouracil
Folex (Methotrexate)
Folex PFS (Methotrexate)
FOLFIRI
FOLFIRI-BEVACIZUMAB
FOLFIRI-CETUXIMAB
FOLFIRINOX
FOLFOX
Folotyn (Pralatrexate)
FU-LV
Fulvestrant
Gardasil (Recombinant HPV Quadrivalent Vaccine)
Gardasil 9 (Recombinant HPV Nonavalent Vaccine)
Gazyva (Obinutuzumab)
Gefitinib
Gemcitabine Hydrochloride
GEMCITABINE-CISPLATIN
GEMCITABINE-OXALIPLATIN
Gemtuzumab Ozogamicin
Gemzar (Gemcitabine Hydrochloride)
Gilotrif (Afatinib Dimaleate)
Gleevec (Imatinib Mesylate)
Gliadel (Carmustine Implant)
Gliadel wafer (Carmustine Implant)
Glucarpidase
Goserelin Acetate
Halaven (Eribulin Mesylate)
Herceptin (Trastuzumab)
HPV Bivalent Vaccine, Recombinant
HPV Nonavalent Vaccine, Recombinant
HPV Quadrivalent Vaccine, Recombinant
Hycamtin (Topotecan Hydrochloride)
Hyper-CVAD
Ibrance (Palbociclib)
Ibritumomab Tiuxetan
Ibrutinib
ICE
Iclusig (Ponatinib Hydrochloride)
Idamycin (Idarubicin Hydrochloride)
Idarubicin Hydrochloride
Idelalisib
Ifex (Ifosfamide)
Ifosfamide
Ifosfamidum (Ifosfamide)
Imatinib Mesylate
Imbruvica (Ibrutinib)
Imiquimod
Inlyta (Axitinib)
Intron A (Recombinant Interferon Alfa-2b)
Iodine 131 Tositumomab and Tositumomab
Ipilimumab
Iressa (Gefitinib)
Irinotecan Hydrochloride
Istodax (Romidepsin)
Ixabepilone
Ixempra (Ixabepilone)
Jakafi (Ruxolitinib Phosphate)
Jevtana (Cabazitaxel)
Kadcyla (Ado-Trastuzumab Emtansine)
Keoxifene (Raloxifene Hydrochloride)
Kepivance (Palifermin)
Keytruda (Pembrolizumab)
Kyprolis (Carfilzomib)
Lanreotide Acetate
Lapatinib Ditosylate
Lenalidomide
Lenvatinib Mesylate
Lenvima (Lenvatinib Mesylate)
Letrozole
Leucovorin Calcium
Leukeran (Chlorambucil)
Leuprolide Acetate
Levulan (Aminolevulinic Acid)
Linfolizin (Chlorambucil)
LipoDox (Doxorubicin Hydrochloride Liposome)
Liposomal Cytarabine
Lomustine
Lupron (Leuprolide Acetate)
Lupron Depot (Leuprolide Acetate)
Lupron Depot-Ped (Leuprolide Acetate)
Lupron Depot-3 Month (Leuprolide Acetate)
Lupron Depot-4 Month (Leuprolide Acetate)
Lynparza (Olaparib)
Marqibo (Vincristine Sulfate Liposome)
Matulane (Procarbazine Hydrochloride)
Mechlorethamine Hydrochloride
Megace (Megestrol Acetate)
Megestrol Acetate
Mekinist (Trametinib)
Mercaptopurine
Mesna
Mesnex (Mesna)
Methazolastone (Temozolomide)
Methotrexate
Methotrexate LPF (Methotrexate)
Mexate (Methotrexate)
Mexate-AQ (Methotrexate)
Mitomycin C
Mitoxantrone Hydrochloride
Mitozytrex (Mitomycin C)
MOPP
Mozobil (Plerixafor)
Mustargen (Mechlorethamine Hydrochloride)
Mutamycin (Mitomycin C)
Myleran (Busulfan)
Mylosar (Azacitidine)
Mylotarg (Gemtuzumab Ozogamicin)
Nanoparticle Paclitaxel (Paclitaxel Albumin-stabilized
Nanoparticle Formulation)
Navelbine (Vinorelbine Tartrate)
Nelarabine
Neosar (Cyclophosphamide)
Neupogen (Filgrastim)
Nexavar (Sorafenib Tosylate)
Nilotinib
Nivolumab
Nolvadex (Tamoxifen Citrate)
Nplate (Romiplostim)
Obinutuzumab
OEPA
Ofatumumab
OFF
Olaparib
Omacetaxine Mepesuccinate
Oncaspar (Pegaspargase)
Ontak (Denileukin Diftitox)
Opdivo (Nivolumab)
OPPA
Oxaliplatin
Paclitaxel
Paclitaxel Albumin-stabilized Nanoparticle Formulation
PAD
Palbociclib
Palifermin
Palonosetron Hydrochloride
Pamidronate Disodium
Panitumumab
Panobinostat
Paraplat (Carboplatin)
Paraplatin (Carboplatin)
Pazopanib Hydrochloride
Pegaspargase
Peginterferon Alfa-2b
PEG-Intron (Peginterferon Alfa-2b)
Pembrolizumab
Pemetrexed Disodium
Perjeta (Pertuzumab)
Pertuzumab
Platinol (Cisplatin)
Platinol-AQ (Cisplatin)
Plerixafor
Pomalidomide
Pomalyst (Pomalidomide)
Ponatinib Hydrochloride
Pralatrexate
Prednisone
Procarbazine Hydrochloride
Proleukin (Aldesleukin)
Prolia (Denosumab)
Promacta (Eltrombopag Olamine)
Provenge (Sipuleucel-T)
Purinethol (Mercaptopurine)
Purixan (Mercaptopurine)
Radium 223 Dichloride
Raloxifene Hydrochloride
Ramucirumab
Rasburicase
R-CHOP
R-CVP
Recombinant Human Papillomavirus (HPV) Bivalent Vaccine
Recombinant Human Papillomavirus (HPV) Nonavalent Vaccine
Recombinant Human Papillomavirus (HPV) Quadrivalent Vaccine
Recombinant Interferon Alfa-2b
Regorafenib
R-EPOCH
Revlimid (Lenalidomide)
Rheumatrex (Methotrexate)
Rituxan (Rituximab)
Rituximab
Romidepsin
Romiplostim
Rubidomycin (Daunorubicin Hydrochloride)
Ruxolitinib Phosphate
Sclerosol Intrapleural Aerosol (Talc)
Siltuximab
Sipuleucel-T
Somatuline Depot (Lanreotide Acetate)
Sorafenib Tosylate
Sprycel (Dasatinib)
STANFORD V
Sterile Talc Powder (Talc)
Steritalc (Talc)
Stivarga (Regorafenib)
Sunitinib Malate
Sutent (Sunitinib Malate)
Sylatron (Peginterferon Alfa-2b)
Sylvant (Siltuximab)
Synovir (Thalidomide)
TAC
Tafinlar (Dabrafenib)
Talc
Tamoxifen Citrate
Tarabine PFS (Cytarabine)
Tarceva (Erlotinib Hydrochloride)
Targretin (Bexarotene)
Tasigna (Nilotinib)
Taxol (Paclitaxel)
Taxotere (Docetaxel)
Temodar (Temozolomide)
Temozolomide
Temsirolimus
Thalidomide
Thalomid (Thalidomide)
Thiotepa
Toposar (Etoposide)
Topotecan Hydrochloride
Toremifene
Torisel (Temsirolimus)
Tositumomab and I 131 Iodine Tositumomab
Totect (Dexrazoxane Hydrochloride)
TPF
Trametinib
Trastuzumab
Treanda (Bendamustine Hydrochloride)
Trisenox (Arsenic Trioxide)
Tykerb (Lapatinib Ditosylate)
Unituxin (Dinutuximab)
VAMP
Vandetanib
Vectibix (Panitumumab)
VeIP
Velban (Vinblastine Sulfate)
Velcade (Bortezomib)
Velsar (Vinblastine Sulfate)
Vemurafenib
VePesid (Etoposide)
Viadur (Leuprolide Acetate)
Vidaza (Azacitidine)
Vinblastine Sulfate
Vincasar PFS (Vincristine Sulfate)
Vincristine Sulfate
Vincristine Sulfate Liposome
Vinorelbine Tartrate
VIP
Vismodegib
Voraxaze (Glucarpidase)
Vorinostat
Votrient (Pazopanib Hydrochloride)
Wellcovorin (Leucovorin Calcium)
Xalkori (Crizotinib)
XELIRI
Xeloda (Capecitabine)
XELOX
Xgeva (Denosumab)
Xofigo (Radium 223 Dichloride)
Xtandi (Enzalutamide)
Yervoy (Ipilimumab)
Zaltrap (Ziv-Aflibercept)
Zelboraf (Vemurafenib)
Zevalin (Ibritumomab Tiuxetan)
Zinecard (Dexrazoxane Hydrochloride)
Ziv-Aflibercept
Zoladex (Goserelin Acetate)
Zoledronic Acid
Zolinza (Vorinostat)
Zometa (Zoledronic Acid)
Zydelig (Idelalisib)
Zykadia (Ceritinib)
Zytiga (Abiraterone Acetate)

### REFERENCES

All publications mentioned herein, are incorporated by reference in their entirety.
1. Dang, C. V., MYC, metabolism, cell growth, and tumorigenesis. Cold Spring Harb Perspect Med, 2013. 3(8).
2. Chisholm, K. M., et al., Expression profiles of MYC protein and MYC gene rearrangement in lymphomas. Am J Surg Pathol, 2015. 39(3): p. 294-303.
3. Sehn, L. H., A decade of R-CHOP. Blood. 116(12): p. 2000-1.
4. Rosenwald, A., et al., The use of molecular profiling to predict survival after chemotherapy for diffuse large-B-cell lymphoma. N Engl J Med, 2002. 346(25): p. 1937-47.
5. Savage, K. J., et al., MYC gene rearrangements are associated with a poor prognosis in diffuse large B-cell lymphoma patients treated with R-CHOP chemotherapy. Blood, 2009. 114(17): p. 3533-7.
6. Barrans, S., et al., Rearrangement of MYC is associated with poor prognosis in patients with diffuse large B-cell lymphoma treated in the era of rituximab. J Clin Oncol, 2010. 28(20): p. 3360-5.
7. Johnson, N. A., et al., Concurrent expression of MYC and BCL2 in diffuse large B-cell lymphoma treated with rituximab plus cyclophosphamide, doxorubicin, vincristine, and prednisone. J Clin Oncol, 2012. 30(28): p. 3452-9.
8. Green, T. M., et al., Immunohistochemical double-hit score is a strong predictor of outcome in patients with diffuse large B-cell lymphoma treated with rituximab plus cyclophosphamide, doxorubicin, vincristine, and prednisone. J Clin Oncol, 2012. 30(28): p. 3460-7.
9. Hu, S., et al., MYC/BCL2 protein coexpression contributes to the inferior survival of activated B-cell subtype of diffuse large B-cell lymphoma and demonstrates high-risk gene expression signatures: a report from The International DLBCL Rituximab-CHOP Consortium Program. Blood, 2013. 121(20): p. 4021-31; quiz 4250.
10. Petrich, A. M., C. Nabhan, and S. M. Smith, MYC-associated and double-hit lymphomas: a review of pathobiology, prognosis, and therapeutic approaches. Cancer, 2014. 120(24): p. 3884-95.
11. Petrich, A. M., et al., Impact of induction regimen and stem cell transplantation on outcomes in double-hit lymphoma: a multicenter retrospective analysis. Blood, 2014. 124(15): p. 2354-61.
12. Lin, P. and L. J. Medeiros, High-grade B-cell lymphoma/leukemia associated with t(14;18) and 8q24/MYC rearrangement: a neoplasm of germinal center immunophenotype with poor prognosis. Haematologica, 2007. 92(10): p. 1297-301.
13. Andresen, C., et al., Transient structure and dynamics in the disordered c-Myc transactivation domain affect Bin1 binding. Nucleic Acids Res, 2012. 40(13): p. 6353-66.
14. Lin, C. J., et al., c-Myc and eIF4F are components of a feedforward loop that links transcription and translation. Cancer Res, 2008. 68(13): p. 5326-34.
15. Wolfe, A. L., et al., RNA G-quadruplexes cause eIF4A-dependent oncogene translation in cancer. Nature, 2014. 513(7516): p. 65-70.
16. Fingar, D. C., et al., Mammalian cell size is controlled by mTOR and its downstream targets S6K1 and 4EBP1/eIF4E. Genes Dev, 2002. 16(12): p. 1472-87.
17. Hutter, G., et al., Proteasome inhibition leads to dephosphorylation and downregulation of protein expression of members of the Akt/mTOR pathway in MCL. Leukemia, 2012. 26(11): p. 2442-4.
18. Quy, P. N., et al., Proteasome-dependent activation of mammalian target of rapamycin complex 1 (mTORC1) is essential for autophagy suppression and muscle remodeling following denervation. J Biol Chem, 2013. 288(2): p. 1125-34.
19. Tang, B., et al., Proteasome Inhibitors Activate Autophagy Involving Inhibition of PI3K-Akt-mTOR Pathway as an Anti-Oxidation Defense in Human RPE Cells. PLoS One, 2014. 9(7): p. e103364.
20. Zhang, Y., et al., Coordinated regulation of protein synthesis and degradation by mTORC1. Nature, 2014. 513(7518): p. 440-3.
21. Pourdehnad, M., et al., Myc and mTOR converge on a common node in protein synthesis control that confers synthetic lethality in Myc-driven cancers. Proc Natl Acad Sci USA, 2013. 110(29): p. 11988-93.
22. Deng, C., et al., The novel IKK2 inhibitor LY2409881 potently synergizes with histone deacetylase inhibitors in preclinical models of lymphoma through the downregulation of NF-kappaB. Clin Cancer Res, 2015. 21(1): p. 134-45.
23. Herman, S. E., et al., Phosphatidylinositol 3-kinase-delta inhibitor CAL-101 shows promising preclinical activity in chronic lymphocytic leukemia by antagonizing intrinsic and extrinsic cellular survival signals. Blood, 2010. 116(12): p. 2078-88.
24. Lannutti, B. J., et al., CAL-101, a p110delta selective phosphatidylinositol-3-kinase inhibitor for the treatment of B-cell malignancies, inhibits PI3K signaling and cellular viability. Blood, 2011. 117(2): p. 591-4.
25. Gopal, A. K., et al., PI3Kdelta inhibition by idelalisib in patients with relapsed indolent lymphoma. The New England journal of medicine, 2014. 370(11): p. 1008-18.
26. Furman, R. R., et al., Idelalisib and Rituximab in Relapsed Chronic Lymphocytic Leukemia. N Engl J Med, 2014.
27. Burris III, H., et al., TGR-1202, a Novel Once Daily P131(6 Inhibitor, Demonstrates Clinical Activity with a Favorable Safety Profile, Lacking Hepatotoxicity, in Patients with CLL and B-Cell Lymphoma. European Hematology Association Annual Meeting, 2015. Abstract and Presentation.
28. Borisy, A. A., et al., Systematic discovery of multicomponent therapeutics. Proc Natl Acad Sci USA, 2003. 100(13): p. 7977-82.
29. Oliver, F. J., et al., Importance of poly(ADP-ribose) polymerase and its cleavage in apoptosis. Lesson from an uncleavable mutant. J Biol Chem, 1998. 273(50): p. 33533-9.
30. Yang, Z., N. He, and Q. Zhou, Brd4 recruits P-TEFb to chromosomes at late mitosis to promote G1 gene expression and cell cycle progression. Mol Cell Biol, 2008. 28(3): p. 967-76.
31. Dey, A., et al., Brd4 marks select genes on mitotic chromatin and directs postmitotic transcription. Mol Biol Cell, 2009. 20(23): p. 4899-909.
32. Filippakopoulos, P., et al., Selective inhibition of BET bromodomains. Nature, 2010. 468(7327): p. 1067-73.
33. Mertz, J. A., et al., Targeting MYC dependence in cancer by inhibiting BET bromodomains. Proc Natl Acad Sci USA, 2011. 108(40): p. 16669-74.
34. Zuber, J., et al., RNAi screen identifies Brd4 as a therapeutic target in acute myeloid leukaemia. Nature, 2011. 478(7370): p. 524-8.
35. Delmore, J. E., et al., BET bromodomain inhibition as a therapeutic strategy to target c-Myc. Cell, 2011. 146(6): p. 904-17.
36. Ott, C. J., et al., BET bromodomain inhibition targets both c-Myc and IL7R in high-risk acute lymphoblastic leukemia. Blood, 2012. 120(14): p. 2843-52.
37. Bordeleau, M. E., et al., Therapeutic suppression of translation initiation modulates chemosensitivity in a mouse lymphoma model. J Clin Invest, 2008. 118(7): p. 2651-60.
38. Cencic, R., et al., Reversing chemoresistance by small molecule inhibition of the translation initiation complex eIF4F. Proc Natl Acad Sci USA, 2011. 108(3): p. 1046-51.
39. Moerke, N.J., et al., Small-molecule inhibition of the interaction between the translation initiation factors eIF4E and eIF4G. Cell, 2007. 128(2): p. 257-67.
40. Boussemart, L., et al., e1F4F is a nexus of resistance to anti-BRAF and anti-MEK cancer therapies. Nature, 2014. 513(7516): p. 105-9.
41. Low, W. K., et al., Second-generation derivatives of the eukaryotic translation initiation inhibitor pateamine A targeting eIF4A as potential anticancer agents. Bioorg Med Chem, 2014. 22(1): p. 116-25.
42. Cencic, R., et al., Antitumor activity and mechanism of action of the cyclopenta[b]benzofuran, silvestrol. PLoS One, 2009. 4(4): p. e5223.
43. Duan, S., et al., mTOR generates an auto-amplification loop by triggering the betaTrCP- and CK1alpha-dependent degradation of DEPTOR. Mol Cell, 2011. 44(2): p. 317-24.
44. Zhao, Y., X. Xiong, and Y. Sun, DEPTOR, an mTOR inhibitor, is a physiological substrate of SCF(betaTrCP) E3 ubiquitin ligase and regulates survival and autophagy. Mol Cell, 2011. 44(2): p. 304-16.
45. Gao, D., et al., mTOR drives its own activation via SCF(betaTrCP)-dependent degradation of the mTOR inhibitor DEPTOR. Mol Cell, 2011. 44(2): p. 290-303.
46. Hutter, G., et al., The proteasome inhibitor bortezomib targets cell cycle and apoptosis and acts synergistically in a sequence-dependent way with chemotherapeutic agents in mantle cell lymphoma. Ann Hematol, 2012. 91(6): p. 847-56.
47. Burris, H. A., et al., TGR-1202, a Novel Once Daily PI3Kδ Inhibitor, Demonstrates Clinical Activity with a Favorable Safety Profile, Lacking Hepatotoxicity, in Patients with Chronic Lymphocytic Leukemia and B-Cell Lymphoma, 2014. 124: 1984-1984.
48. O'Connor, O. A., et al., A phase 1 dose escalation study of the safety and pharmacokinetics of the novel proteasome inhibitor carfilzomib (PR-171) in patients with hematologic malignancies. Clinical cancer research: an official journal of the American Association for Cancer Research, 2009. 15(22): p. 7085-91.
49. Cegielska, A., Gietzen, K. F., Rivers, A., and Virshup, D. M. (1998). Autoinhibition of casein kinase I epsilon (CKI epsilon) is relieved by protein phosphatases and limited proteolysis. J Biol Chem 273: 1357-1364.
50. Cheong, J. K., Nguyen, T. H., Wang, H., Tan, P., Voorhoeve, P. M., Lee, S. H., and Virshup, D. M. (2011). IC261 induces cell cycle arrest and apoptosis of human cancer cells via CK1delta/varepsilon and Wnt/beta-catenin independent inhibition of mitotic spindle formation. Oncogene 30: 2558-2569.
51. Rivers, A., Gietzen, K. F., Vielhaber, E., and Virshup, D. M. (1998). Regulation of casein kinase I epsilon and casein kinase I delta by an in vivo futile phosphorylation cycle. J Biol Chem 273: 15980-15984.
52. Deng, C., McIntosh, C., Rodriguez, R., Sportelli, P., Miskin, H. P., Vakkalanka, S., Viswanadha, S., Lipstein, M., and O'Connor, O. A. (2013). The PI3K Delta Inhibitor TGR-1202 and Proteasome Inhibitor Carfilzomib Are Highly Synergistic In Killing Human B- and T-Cell Lymphoma Cells. Blood 122: 4421.
53. Long, A. M., Zhao, H., and Huang, X. (2012). Structural basis for the potent and selective inhibition of casein kinase 1 epsilon. J Med Chem 55: 10307-10311.

1. A method for treating a c-Myc-overexpressing cancer in a subject comprising co-administering a therapeutically effective amount of a dual PI3K/CK-1 inhibitor with a therapeutically effective amount of a proteasome inhibitor, or optionally, co-administering a therapeutically effective amount of a PI3K inhibitor, a CK-1 inhibitor and a proteasome inhibitor.

2. The method of claim 1, wherein the cancer is a hematological cancer.

3. The method of claim 1, wherein the cancer is a B cell cancer.

4. The method of claim 1, wherein the B cell cancer is multiple myeloma or lymphoma.

5. The method of claim 1, wherein the cancer is cancer solid tumor in an organ selected from the group consisting of the lung, breast, prostate, ovary, colon, kidney, and liver.

6. The method of claim 1, wherein the PI3K inhibitor comprises TGR-1202, or an therapeutically active analog or derivative thereof, or pharmaceutically acceptable salt of any of the foregoing.

7. The method of claim 1, wherein the proteasome inhibitor comprises carfilzomib, or an therapeutically active analog or derivative thereof, or pharmaceutically acceptable salt of any of the foregoing.

8. The method of claim 1, wherein the dual PI3K/CK-1 inhibitor comprises CK-1ε, CK-1α, or CK-1δ inhibitory activity.

9. The method of claim 1, wherein the dual PI3K/CK1 inhibitor comprises CK-1ε inhibitory activity.

10. The method of claim 1, wherein CK-1 inhibitor inhibits CK-1ε, CK-1α, or CK-1δ.

11. A method comprising:
(a) determining a CK-1 expression level from a cancer cell sample obtained from a subject who has cancer; and
(b) comparing the expression level from the cancer cell sample to an expression level of a control, wherein an elevated CK-1 expression level in the cancer cell sample relative to the control indicates that the cancer is susceptible to PI3K and CK-1 inhibition; and if the cancer is susceptible, co-administering a therapeutically effective amount of a dual PI3K/CK-1 inhibitor with a therapeutically effective amount of a proteasome inhibitor, or optionally, co-administering a therapeutically effective amount of a PI3K inhibitor, CK-1 inhibitor and proteasome inhibitor.

12. The method of claim 11, wherein the dual PI3K/CK-1 inhibitor is TGR-1202 or CUX-03173 a therapeutically active analog or derivative thereof, or a pharmaceutically acceptable salt of any of the foregoing; and the proteasome inhibitor is carfilzomib, or a therapeutically active analog or derivative thereof, or a pharmaceutically acceptable salt of any of the foregoing.

13. The method of claim 11, wherein the cancer is a c-Myc-overexpressing cancer.

14. The method of claim 13, wherein the cancer is a B cell cancer.

15. The method of claim 14, wherein the B cell cancer is multiple myeloma or lymphoma.

16. The method of claim 11, wherein the expression level is selected from the group consisting of RNA transcript level and protein level.

17. The method of claim 11, wherein the CK-1 is selected from the group consisting of CK-1ε, CK-1α, or CK-1δ.

18. The method of claim 11, wherein the CK-1 is CK-1α, and the cancer is selected from the group consisting of lung cancer, colon cancer, and liposarcoma.

19. The method of claim 11, wherein the CK-1 is CK-1δ and the cancer is selected from the group consisting of lung cancer, choriocarcinoma, high-grade ductal pancreatic carcinoma and glioblastoma.

20. The method of claim 11, wherein the CK-1 is CK-1ε and the cancer is selected from the group consisting of B cell cancer, lung cancer, breast cancer, adenoid cystic carcinoma, epithelial ovarian cancer, renal cancer, bladder cancer, prostate cancer, melanoma and seminoma.

21. A method comprising
contacting a known PI3K inhibitor candidate agent with a CK-1 isoform, to produce a test sample;
determining level of CK-1 isoform activity in test sample; and
if the CK-1 isoform activity is reduced, selecting the PI3K candidate agent as having a dual function of also inhibiting CK-1.

22. A pharmaceutical formulation comprising:
a therapeutically effective amount of a dual PI3K/CK-1 inhibitor; and
a therapeutically effective amount of a proteasome inhibitor; and optionally
a pharmaceutically acceptable carrier.

23. The formulation of claim 22, wherein the proteasome inhibitor is carfilzomib, or an therapeutically active analog or derivative thereof, or a pharmaceutically acceptable salt of any of the foregoing.

24. A pharmaceutical formulation comprising:
a therapeutically effective amount of a PI3k inhibitor;
a therapeutically effective amount of a CK-1 inhibitor; and
a therapeutically effective amount of a proteasome inhibitor; and optionally
a pharmaceutically acceptable carrier.

25. A pharmaceutical formulation comprising:
(i) a therapeutically effective amount of a dual PI3K/CK-1 inhibitor and therapeutically effective amount of a proteasome inhibitor;
ii) a therapeutically effective amount of a PI3K-AKT-mTOR signaling pathway inhibitor inhibitor, a therapeutically effective amount of a CK-1 inhibitor, and a therapeutically effective amount of a proteasome inhibitor;
iii) a therapeutically effect amount of a dual PI3K/CK-1 inhibitor, a therapeutically effect amount of a CK-1 inhibitor and a therapeutically effect amount of proteasome inhibitor;
iv) a therapeutically effect amount of a dual PI3K/CK-1 inhibitor and a therapeutically effect amount of an adjunct cancer therapeutic agent (excluding a proteasome inhibitor); or
v) a therapeutically effect amount of a PI3K-AKT-mTOR signaling pathway inhibitor, a therapeutically effect amount of a CK-1 inhibitor and a therapeutically effect amount of an adjunct cancer therapeutic agent (excluding a proteasome inhibitor); and
optionally, wherein i-v are further combined with a pharmaceutically acceptable carrier.

26. A method for treating a c-Myc-overexpressing cancer in a subject comprising administering a c-Myc reducing amount of a CK-1 epsilon inhibitor or a dual PI3K/CK-1 inhibitor, or both; and optionally co-administering a therapeutically effective amount of a proteasome inhibitor or a PI3K inhibitor, or both.

27. The method of claim 26, wherein the cancer is a hematological cancer.

28. The method of claim 26, wherein the cancer is a B cell cancer.

29. The method of claim 28, wherein the B cell cancer is multiple myeloma or lymphoma.

30. The method of claim 26, wherein the cancer is cancer solid tumor in an organ selected from the group consisting of the lung, breast, prostate, ovary, colon, kidney, and liver.

31. The method of claim 1, wherein the PI3K inhibitor comprises Idelalisib or develisib, or a therapeutically active therapeutically active analog or derivative thereof, or pharmaceutically acceptable salt thereof of the foregoing.

32. The method of claim 26, wherein the dual PI3K/CK-1 inhibitor is selected from the group consisting of TGR-1202 and CUX-03173; or a therapeutically active therapeutically active analog or derivative thereof, or pharmaceutically acceptable salt thereof of the foregoing.

33. The method of claim 26, wherein the proteasome inhibitor comprises carfilzomib, or an therapeutically active analog or derivative thereof, or pharmaceutically acceptable salt thereof of the foregoing.

34. The method of claim 26, wherein the CK-1 inhibitor comprises CK-1ε, CK-1α, or CK-1δ inhibitory activity, or a combination thereof.

35. The method of claim 26, wherein the CK-1 inhibitor comprises CK-1ε inhibitory activity.

36. A method for treating a c-Myc-overexpressing cancer in a subject comprising administering an agent according to Formula III or Formula IV, or a pharmaceutically acceptable salt thereof: 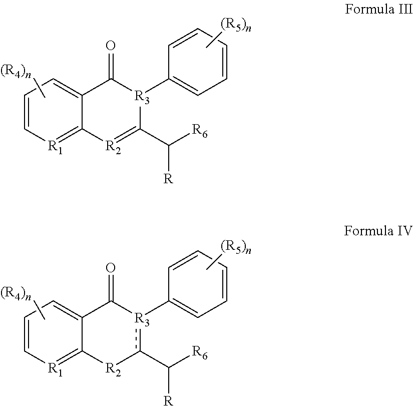
wherein
R is H or any one of groups A-G: 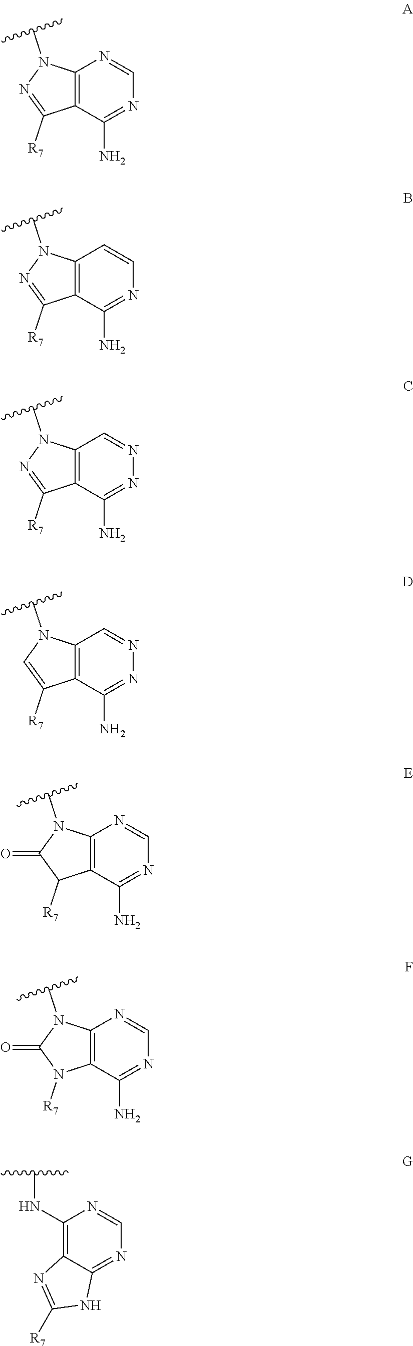
and wherein [symbol]
represents a single or double bond;
R₁ is CH, substituted C or N;
R₂
in the compound of Formula III is CH, substituted C or N;
in the compound of Formula IV is O, CH₂, substituted C, NH or substituted N;
R₃
in the compound of Formula III is CH, substituted C or N;
in the compound of Formula IV is
CH, substituted C or N when [symbol] represents a single bond; or
C when [symbol] represents a double bond;
each R₄ is independently substituted alkyl, unsubstituted alkyl, substituted alkenyl, unsubstituted alkenyl, substituted alkynyl, unsubstituted alkynyl, or halogen;
each R₅ is independently substituted alkyl, unsubstituted alkyl, substituted alkenyl, unsubstituted alkenyl, substituted alkynyl, unsubstituted alkynyl, or halogen;
R₆ is H, Me or Me substituted with halogen;
R₇ is H or a group selected from any one of groups J, K and H 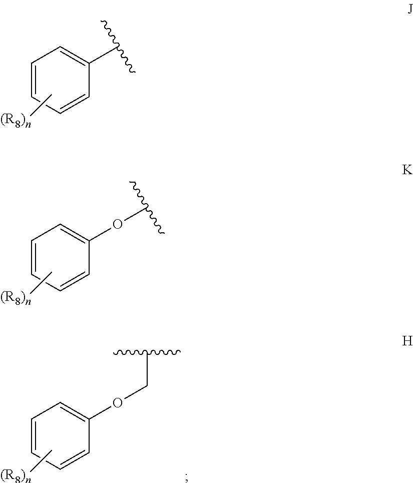
and
each R₈ is independently substituted alkyl, unsubstituted alkyl, substituted O-alkyl, unsubstituted O-alkyl or halogen;
n,
for R₄ and when R₁ is not N, is 0, 1, 2, 3 or 4;
for R₄ and when R₁ is N, is 0, 1, 2 or 3;
for R₅ is 0, 1, 2, 3, 4 or 5;
for R₈ is 0, 1, 2, 3, 4 or 5;
wherein the compound according to Formula III or Formula IV is administered at a CK-1 reducing effective amount.

37. The method of claim 36, further comprising the proviso that compounds of formula III wherein at the same time R is group A, R₁ is CH, R₃ is N and R₇ is J, are excluded.

38. The method of claim 36, further comprising the proviso that compounds of formula IV wherein at the same time R is group A, R₁ is CH, R₂ is O, R₃ is C, [symbol] represents a double bond, and R₇ is J, are excluded.

39. The method of claim 36, further comprising the proviso that R₇ is not H when R is group G.

40. The method of claim 36, further comprising the provisos that
compounds of formula III wherein at the same time R is group A, R₁ is CH, R₃ is N and R₇ is J, are excluded;
compounds of formula IV wherein at the same time R is group A, R₁ is CH, R₂ is O, R₃ is C, [symbol] represents a double bond, and R₇ is J, are excluded;
R₇ is not H when R is group G.

41. The method of claim 36, wherein R₁ is N.

42. The method of claim 36, wherein R₂ is not O.

43. The method of claim 36, wherein R₃ is not N.

44. The method of claim 36, wherein R₄ is halogen and n for R₄ is 1 or 2.

45. The method of claim 36, wherein R₄ is F and n for R₄ is 1 or 2.

46. The method of claim 36, wherein the CK-1epsilon inhibitor is 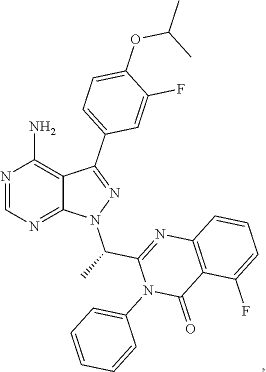 [SMILES: CC(C)Oc1ccc(-c2nn([C@@H](C)c3nc4cccc(F)c4c(=O)n3-c3ccccc3)c3ncnc(N)c23)cc1F ; ","]
or a pharmaceutically acceptable salt thereof.

47. The method of claim 36, wherein R₄ is F, n for R₄ is 1, and R₄ is located at position 5 of the quinazolin-4-one ring to which it is attached.

48. The method of claim 36, wherein n for R₅ is 0.

49. The method of claim 36, wherein R₆ is Me.

50. The method of claim 36, wherein R is not group A.

51. The method of claim 36, wherein R is group A.

52. The method of claim 36, wherein R₇ is J.

53. The method of claim 36, wherein R₇ is not J.

54. The method of claim 36, wherein n for R₈ is 2, one R₈ is isopropyl or O-isopropyl, and the other R₈ is halogen.

55. The method of claim 36, wherein R₇ is one of the following:

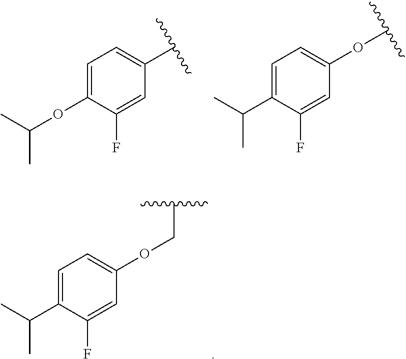 [SMILES: *c1ccc(OC(C)C)c(F)c1 ; *Oc1ccc(C(C)C)c(F)c1 ; *COc1ccc(C(C)C)c(F)c1 ; "."]

56. A method comprising:
(a) determining a CK-1 expression level from a cancer cell sample obtained from a subject who has cancer;
(b) comparing the expression level from the cancer cell sample to an expression level of a control, wherein an elevated CK-1 expression level in the cancer cell sample relative to the control indicates that the cancer is susceptible to CK-1 inhibition; and administering a therapeutically effective amount of a CK-1 inhibitor or dual PI3K/CK-1 inhibitor, or both, to a susceptible cancer.

57. The method of claim 56, further comprising co-administering a therapeutically effective amount of a proteasome inhibitor, or a therapeutically effective amount of a PI3K inhibitor, or both.

58. The method of claim 56, wherein the cancer is a c-Myc-overexpressing cancer.

59. The method of claim 56, wherein the cancer is a B cell cancer.

60. The method of claim 59, wherein the B cell cancer is multiple myeloma or lymphoma.

61. The method of claim 56, wherein the expression level is selected from the group consisting of RNA transcript level and protein level.

62. The method of claim 56, wherein the CK-1 is selected from the group consisting of CK-1ε, CK-1α, or CK-1δ.

63. The method of claim 56, wherein the CK-1 is CK-1α, and the cancer is selected from the group consisting of lung cancer, colon cancer, and liposarcoma.

64. The method of claim 56, wherein the CK-1 is CK-1δ and the cancer is selected from the group consisting of lung cancer, choriocarcinoma, high-grade ductal pancreatic carcinoma and glioblastoma.

65. The method of claim 56, wherein the CK-1 is CK-1ε and the cancer is selected from the group consisting of B cell cancer, lung cancer, breast cancer, adenoid cystic carcinoma, epithelial ovarian cancer, renal cancer, bladder cancer, prostate cancer, melanoma and seminoma.

66. A method comprising:
a) determining a pre-treatment CK-1 expression level in a first cancer cell sample from a subject that has cancer;
b) co-administering a therapeutically effective amount of a dual PI3K/CK-1 inhibitor with a therapeutically effective amount of a proteasome inhibitor, or optionally, co-administering a therapeutically effective amount of a PI3K inhibitor, CK-1 inhibitor and proteasome inhibitor; and
c) determining a post-treatment CK-1 expression level in a second cancer cell sample from the subject;
wherein a reduction in the post-treatment CK-1 expression level relative to the pre-treatment level indicates that the co-administration chemotherapy is effective to treat the cancer.

67. The method of claim 66, wherein the CK-1 is selected from the group consisting of CK-1ε, CK-1α, or CK-1δ.

68. The method of claim 66, further comprising repeating step (b) if a reduction in the post-treatment CK-1 expression level is determined.

69. A compound according to Formula III or Formula IV: 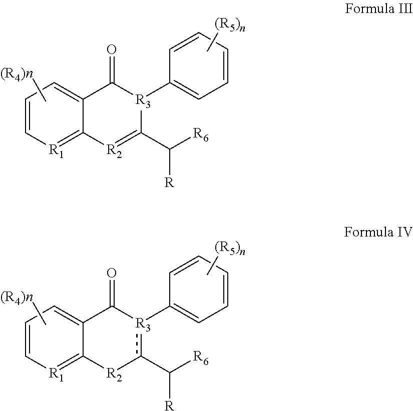
wherein
R is H or any one of groups A-G: 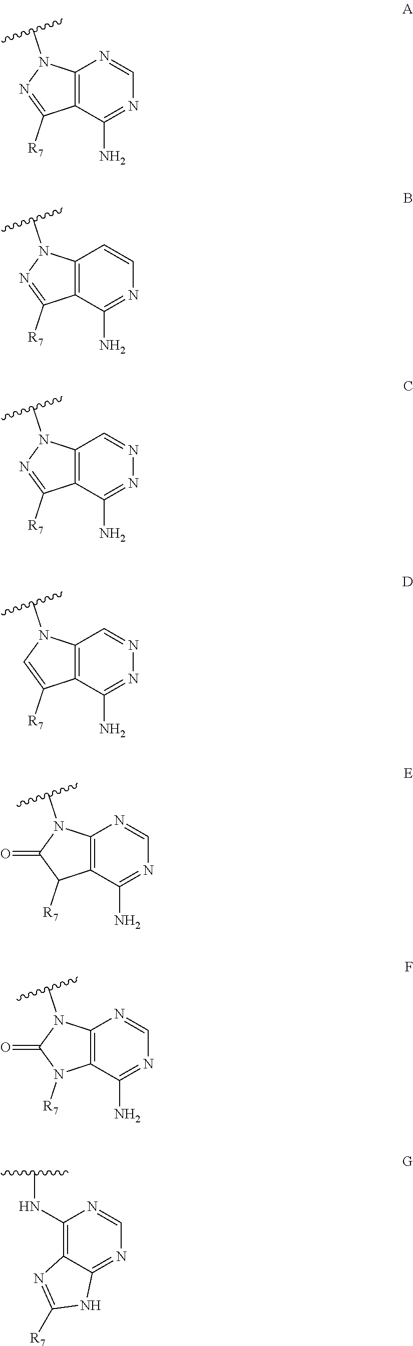
and wherein [symbol]
represents a single or double bond;
R₁ is CH, substituted C or N;
R₂
in the compound of Formula III is CH, substituted C or N;
in the compound of Formula IV is O, CH₂, substituted C, NH or substituted N;
R₃
in the compound of Formula III is CH, substituted C or N;
in the compound of Formula IV is
CH, substituted C or N when [symbol] represents a single bond; or
C when [symbol] represents a double bond;
each R₄ is independently substituted alkyl, unsubstituted alkyl, substituted alkenyl, unsubstituted alkenyl, substituted alkynyl, unsubstituted alkynyl, or halogen;
each R₅ is independently substituted alkyl, unsubstituted alkyl, substituted alkenyl, unsubstituted alkenyl, substituted alkynyl, unsubstituted alkynyl, or halogen;
R₆ is H, Me or Me substituted with halogen;
R₇ is H or a group selected from any one of groups J, K and H 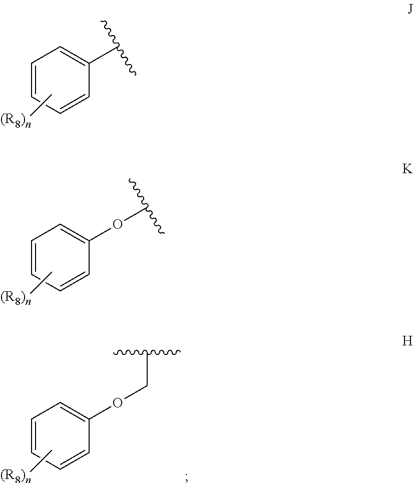
and
each R₈ is independently substituted alkyl, unsubstituted alkyl, substituted O-alkyl, unsubstituted O-alkyl or halogen;
n,
for R₄ and when R₁ is not N, is 0, 1, 2, 3 or 4;
for R₄ and when R₁ is N, is 0, 1, 2 or 3;
for R₅ is 0, 1, 2, 3, 4 or 5;
for R₈ is 0, 1, 2, 3, 4 or 5;
further comprising the provisos that
(i) compounds of formula III wherein at the same time R is group A, R₁ is CH, R₃ is N and R₇ is J, are excluded;
(ii) compounds of formula IV wherein at the same time R is group A, R₁ is CH, R₂ is O, R₃ is C, [symbol] represents a double bond, and R₇ is J, are excluded;
(iii) R₇ is not H when R is group G.

70. The compound of claim 69, wherein R₁ is N.

71. The compound of claim 69 wherein R₂ is not O.

72. The compound of claim 69 wherein R₃ is not N.

73. The compound of claim 69 wherein R₄ is halogen and n for R₄ is 1 or 2.

74. The compound of claim 69 wherein R₄ is F and n for R₄ is 1 or 2.

75. The compound of claim 69 wherein R₄ is F, n for R₄ is 1, and R₄ is located at position 5 of the quinazolin-4-one ring to which it is attached.

76. The compound of claim 69 wherein n for R₅ is O.

77. The compound of claim 69 wherein R₆ is Me.

78. The compound of claim 69 wherein R is not group A.

79. The compound of claim 69 wherein R is group A.

80. The compound of claim 69 wherein R₇ is J.

81. The compound of any of claim 69 wherein R₇ is not J.

82. The compound of claim 69 wherein n for R₈ is 2, one R₈ is isopropyl or O-isopropyl, and the other R₈ is halogen.

83. The compound of claim 69 wherein R₇ is one of the following:

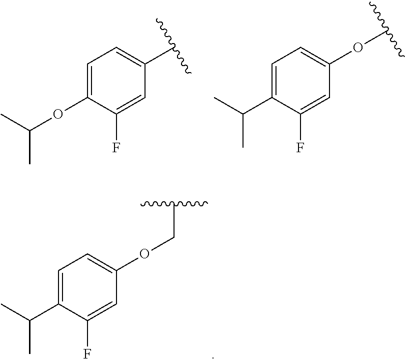 [SMILES: *c1ccc(OC(C)C)c(F)c1 ; *Oc1ccc(C(C)C)c(F)c1 ; *COc1ccc(C(C)C)c(F)c1 ; "."]

84. A kit for administering a first and a second pharmaceutical composition to a subject suffering from a c-Myc-overexpressing cancer, the kit comprising: i) a plurality of separate containers, the contents of at least two containers differing from each other in whole or in part, wherein at least one of such containers contains a CK-1 inhibitor or a dual PI3K/CK-1 inhibitor, or both, with or without additional pharmaceutical carrier or diluent, and at least one different container contains a proteasome inhibitor, with or without additional pharmaceutical carrier or diluent; or at least one different container contains a PI3K inhibitor, with or without additional pharmaceutical carrier or diluent and, optionally, ii) instructions for the use of the contents of the containers after an interval of time has passed after administration of the first pharmaceutical composition for the treatment of a subject suffering from a hematological cancer.

85. A method comprising:
administering to a c-Myc-overexpressing cell in a subject a c-myc reducing amount of a CK-1 inhibitor or dual PI3K/CK-1 inhibitor; and
administering an adjunct cancer therapy protocol in the subject.

86. The method of claim 85, wherein the adjunct cancer therapy protocol comprises co-administration of an adjunct cancer therapeutic agent.

87. The method of claim 86, wherein the adjunct cancer therapeutic agent is co-administered upon reduction of c-Myc in the c-Myc-overexpressing cell by CK-1 inhibitor administration.

88. The method of claim 86, wherein the adjunct cancer therapeutic agent excludes a proteasome inhibitor.

89. A method comprising:
administering to a subject a therapeutically effective amount of a CK-1epsilon inhibitor; or
co-administering (i) a therapeutically effective amount of an mTOR inhibitor and a therapeutically effective amount of a proteasome inhibitor; (ii) a therapeutically effective amount of an mTOR inhibitor and a therapeutically effective amount of a CK-1epsilon inhibitor; or (iii) a therapeutically effective amount of CK-1epsilon inhibitor and a therapeutically effective amount of a proteasome inhibitor;
wherein the subject has received an organ transplant.

90. The method of claim 89, wherein the subject is at risk of GVHD related to the organ transplant, or exhibits symptoms of GVHD.

91. The method of claim 65, wherein the organ transplant is a bone marrow transplant.

92. A method comprising
administering to a subject a therapeutically effective amount of an agent that inhibits CK-1epsilon; or
co-administering (i) a therapeutically effective amount of an mTOR inhibitor and a therapeutically effective amount of a proteasome inhibitor; (ii) a therapeutically effective amount of an mTOR inhibitor and a therapeutically effective amount of an agent that inhibits CK-1epsilon; or (iii) a therapeutically effective amount of an agent that inhibits CK-1epsilon and a therapeutically effective amount of a proteasome inhibitor;
wherein the subject exhibits symptoms of and/or has been diagnosed with an autoimmune disease.

93. The method of claim 92, wherein the autoimmune disease is rheumatoid arthritis, psoriasis, eczema, asthma, multiple sclerosis, inflammatory bowel disease, Chrohn's disease, colitis, systemic lupus erythematosus, myasthenia gravis, Sjögren's syndrome and sclerodema, autoimmune hemolytic anemia, cold agglutinin disease, or IgA nephropathy.

94. The method of claim 92, wherein the proteasome inhibitor comprises carfilzomib, or a pharmaceutically acceptable salt thereof.

95. The method of claim 92, wherein the agent that reduces CK-1 epsilon is an agent according to Formula III or Formula IV, or a pharmaceutically acceptable salt thereof: 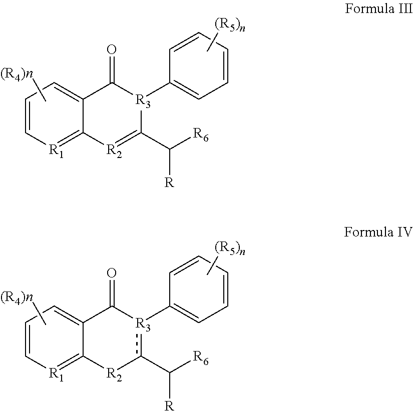
wherein
R is H or any one of groups A-G: 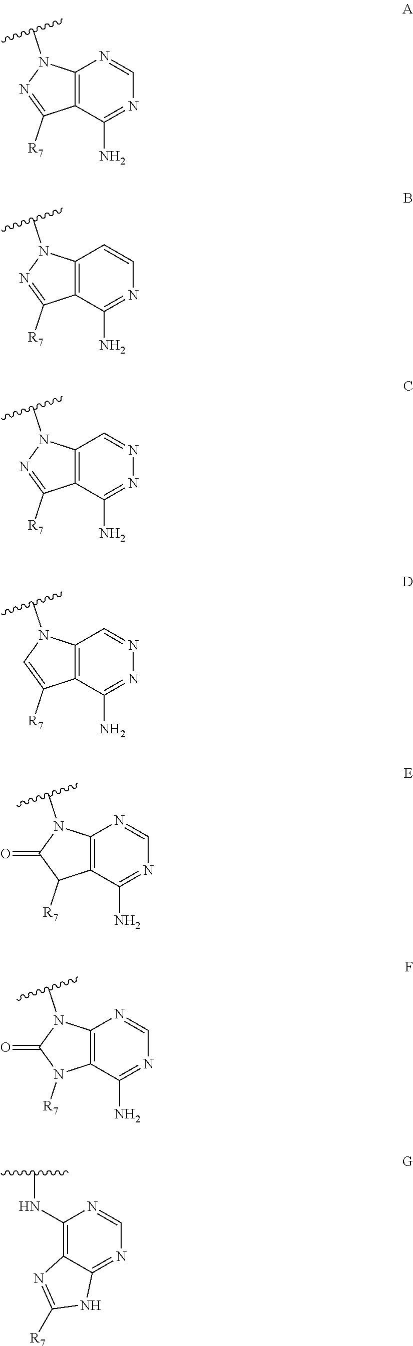
and wherein [symbol]
represents a single or double bond;
R₁ is CH, substituted C or N;
R₂
in the compound of Formula III is CH, substituted C or N;
in the compound of Formula IV is O, CH₂, substituted C, NH or substituted N;
R₃
in the compound of Formula III is CH, substituted C or N;
in the compound of Formula IV is
CH, substituted C or N when [symbol] represents a single bond; or
C when [symbol] represents a double bond;
each R₄ is independently substituted alkyl, unsubstituted alkyl, substituted alkenyl, unsubstituted alkenyl, substituted alkynyl, unsubstituted alkynyl, or halogen;
each R₅ is independently substituted alkyl, unsubstituted alkyl, substituted alkenyl, unsubstituted alkenyl, substituted alkynyl, unsubstituted alkynyl, or halogen;
R₆ is H, Me or Me substituted with halogen;
R₇ is H or a group selected from any one of groups J, K and H 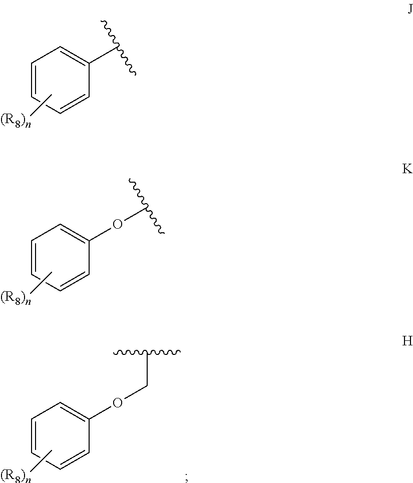
and
each R₈ is independently substituted alkyl, unsubstituted alkyl, substituted O-alkyl, unsubstituted O-alkyl or halogen;
n,
for R₄ and when R₁ is not N, is 0, 1, 2, 3 or 4;
for R₄ and when R₁ is N, is 0, 1, 2 or 3;
for R₅ is 0, 1, 2, 3, 4 or 5;
for R₈ is 0, 1, 2, 3, 4 or 5;
wherein the compound according to Formula III or Formula IV is administered at a CK-1 reducing effective amount.

96. The method of claim 95, wherein the agent that inhibits CK-1 epsilon is 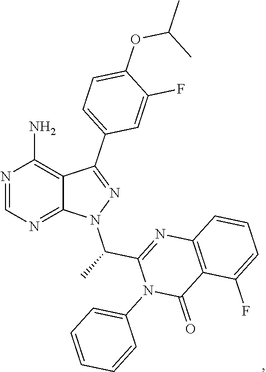 [SMILES: CC(C)Oc1ccc(-c2nn([C@@H](C)c3nc4cccc(F)c4c(=O)n3-c3ccccc3)c3ncnc(N)c23)cc1F ; ","]
or a pharmaceutically acceptable salt thereof; or 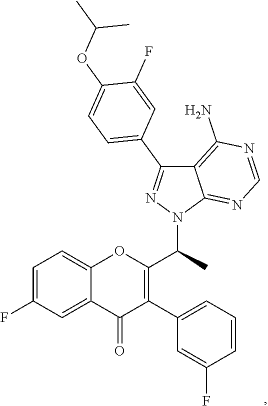 [SMILES: CC(C)Oc1ccc(-c2nn([C@@H](C)c3oc4ccc(F)cc4c(=O)c3-c3cccc(F)c3)c3ncnc(N)c23)cc1F ; ","]
or a pharmaceutically acceptable salt thereof.


SEQUENCE LISTING (4 sequences)

SEQ ID NO: 1 - protein, 416 aa, Homo sapiens
  misc_feature: Casein Kinase 1 (epsilon) human protein
MELRVGNKYRLGRKIGSGSFGDIYLGANIASGEEVAIKLECVKTKHPQLHIESKFYKMMQGGVGIPSIKWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFSTYLNFCRSLRFDDKPDYSYLRQLFRNLFHRQGFSYDYVFDWNMLKFGAARNPEDVDRERREHEREERMGQLRGSATRALPPGPPTGATANRLRSAAEPVASTPASRIQPAGNTSPRAISRVDRERKVSMRLHRGAPANVSSSDLTGRQEVSRIPASQTSVPFDHLGK

SEQ ID NO: 2 - DNA, 2670 bp, Homo sapiens
  misc_feature: Casein Kinase 1 (epsilon) human mRNA
ggttgggatctgaggggtcctctctgtgcccatcacagtttgagcttcagggaaaagaagaagaggtctttgcccttcgtttttccacgggaggagaatcaagagtgagccatggagctacgtgtggggaacaagtaccgcctgggacggaagatcgggagcgggtccttcggagatatctacctgggtgccaacatcgcctctggtgaggaagtcgccatcaagctggagtgtgtgaagacaaagcacccccagctgcacatcgagagcaagttctacaagatgatgcagggtggcgtggggatcccgtccatcaagtggtgcggagctgagggcgactacaacgtgatggtcatggagctgctggggcctagcctcgaggacctgttcaacttctgttcccgcaaattcagcctcaagacggtgctgctcttggccgaccagatgatcagccgcatcgagtatatccactccaagaacttcatccaccgggacgtcaagcccgacaacttcctcatggggctggggaagaagggcaacctggtctacatcatcgacttcggcctggccaagaagtaccgggacgcccgcacccaccagcacattccctaccgggaaaacaagaacctgaccggcacggcccgctacgcttccatcaacacgcacctgggcattgagcaaagccgtcgagatgacctggagagcctgggctacgtgctcatgtacttcaacctgggctccctgccctggcaggggctcaaagcagccaccaagcgccagaagtatgaacggatcagcgagaagaagatgtcaacgcccatcgaggtcctctgcaaaggctatccctccgaattctcaacatacctcaacttctgccgctccctgcggtttgacgacaagcccgactactcttacctacgtcagctcttccgcaacctcttccaccggcagggcttctcctatgactacgtctttgactggaacatgctgaaattcggtgcagcccggaatcccgaggatgtggaccgggagcggcgagaacacgaacgcgaggagaggatggggcagctacgggggtccgcgacccgagccctgccccctggcccacccacgggggccactgccaaccggctccgcagtgccgccgagcccgtggcttccacgccagcctcccgcatccagccggctggcaatacttctcccagagcgatctcgcgggtcgaccgggagaggaaggtgagtatgaggctgcacaggggtgcgcccgccaacgtctcctcctcagacctcactgggcggcaagaggtctcccggatcccagcctcacagacaagtgtgccatttgaccatctcgggaagtgaggagagcccccattggaccagtgtttgcttagtgtcttcactgtattttctttaaaaaaaaaaaaaaaaaaaaaaaggcaaaaataaaccactcaaaagaacaacaaaaaaacccagcacaaaaccgacgatggagtttgtttctttgatttctttgccaatggcaagaagatgagatgccctcagcactgaggattcttgcccccttgtggtgcccgctgcccccaaccttcaggctgccagatgctcccctgacaacaccaggctacaggagccagacgccagggcctgcccggcctcctgttcctgcccccacccaccacctgcctggagaggaacgggtcgggtccgtgtcggagaagtgacaggtcccagagccaaagccggccctcaagcatcatcagggagtggtgtagtcagttgaaggcagttcccaccgagttttccgagcctcagaatccaggagatacgcacagccccacccactctgagatgacagtggctgacttcccgtgctgggcttttccattgtccccctggcctccaggctcctcctctgcctctccatggagtgggtggggaggtggtgggggccggcgtcccctgcgtgtgtgtgtgtgtgtgtgtgtgtggatgtattgacctgtgtttcccaagacagcaggtgccacggcccgccccgcctgccagcccgaattcccgttctcctgtgtctactaacaaggacatgggggtgggcggtgacctccgcatccctcagagctcagagggtcctcgctgccaccggtccccccctagcccgtcatcagccggtggcagctccatcttccattcctggttttagggcagaatccatggagactgcttccagaaggcatctggctctgagttataaattacttccctggtcctgacagtcacctggggtcccccctctccctggttccacctttctgaggaggagcctggagtcagggctgggttttggattaacccatccttcctagttaacacctttttgtttttattttattttatttttgtttgttttctccgtgtgtgtgttttcctaatttatttacctctgtttcccctttttcctttttttttttaattaaagagcaaagctttttattactttgtaatttaaaaaactgaaaaaaaaaaaactgaagaactttggggggaattttgtacttttttcctgtgtaaatattggacttttttgagctttatcgtggttgttaatttgaagtaataaagtagaaaagataaagtgaaaaaaaaaaaaaaaaaaaaaaaaaaaaa

SEQ ID NO: 3 - protein, 1044 aa, Homo sapiens
  misc_feature: phosphatidylinositol 4,5-bisphosphate 3-kinase catalytic subunit delta isoform, or PI3K-delta, human protein
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

SEQ ID NO: 4 - protein, 14 aa, Artificial Sequence
  Synthetic CK1 peptide
  MOD_RES (7)..(7): Phosphoserine
KRRRALSVASLPGL